SEQUENCE LISTING (266 sequences)

SEQ ID NO: 1 - DNA, 2583 bp, Homo sapiens
atggggccctggggctggaaattgcgctggaccgtcgccttgctcctcgccgcggcggggactgcagtgggcgacagatgtgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagggacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaatgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggtcctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccggcctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcgtga

SEQ ID NO: 2 - protein, 860 aa, Homo sapiens [sequence omitted]

SEQ ID NO: 3 - DNA, 252 bp, Homo sapiens
gggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctca

SEQ ID NO: 4 - protein, 84 aa, Homo sapiens
GTNECLDNNGGCSHVCNDLKIGYECLCPDGFQLVAQRRCEDIDECQDPDTCSQLCVNLEGGYKCQCEEGFQLDPHTKACKAVGS

SEQ ID NO: 5 - DNA, 36 bp, Artificial Sequence
  Synthetic oligonucleotide
accggtggagggaccaacgaatgcttggacaacaac

SEQ ID NO: 6 - DNA, 39 bp, Artificial Sequence
  Synthetic oligonucleotide
ctcgagatctgagcccacagccttgcaggccttcgtgtg

SEQ ID NO: 7 - DNA, 22 bp, Artificial Sequence
  Synthetic oligonucleotide
ggctgttcctacgtctgcaatg

SEQ ID NO: 8 - DNA, 22 bp, Artificial Sequence
  Synthetic oligonucleotide
cattgcagacgtaggaacagcc

SEQ ID NO: 9 - DNA, 30 bp, Artificial Sequence
  Synthetic oligonucleotide
cccgggaccaacgaatgcttggacaacaac

SEQ ID NO: 10 - DNA, 42 bp, Artificial Sequence
  Synthetic oligonucleotide
tctagattatcatgagcccacagccttgcaggccttcgtgtg

SEQ ID NO: 11 - DNA, 49 bp, Artificial Sequence
  Synthetic oligonucleotide
tctccaccgtctccagcacctgaactcctgggtggatcgtcagtcttcc

SEQ ID NO: 12 - DNA, 49 bp, Artificial Sequence
  Synthetic oligonucleotide
agatctcgagcccaaatcttctgacaaaactcacacatctccaccgtct

SEQ ID NO: 13 - DNA, 51 bp, Artificial Sequence
  Synthetic oligonucleotide
tctagattatcatttacccggagagagagagaggctcttctgcgtgtagtg

SEQ ID NO: 14 - DNA, 49 bp, Artificial Sequence
  Synthetic oligonucleotide
agatctcgagcccaaatcttctgacaaaactcacacatgtccaccgtgt

SEQ ID NO: 15 - DNA, 39 bp, Artificial Sequence
  Synthetic oligonucleotide
gttttctcgatggaggctgggagggctttgttggagacc

SEQ ID NO: 16 - DNA, 48 bp, Artificial Sequence
  Synthetic oligonucleotide
aaggtctccaacaaagccctcccagcctccatcgagaaaacaatctcc

SEQ ID NO: 17 - DNA, 1045 bp, Artificial Sequence
  hVK3LP-hLDLR-EGFAB-H306Y-(G4S)4-sssmthIgG nucleotide
cggtggagggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 18 - protein, 363 aa, Artificial Sequence
  hVK3LP-hLDLR-EGFAB-H306Y-(G4S)4-sssmthIgG peptide
METPAQLLFLLLLWLPDTTGGGTNECLDNNGGCSYVCNDLKIGYECLCPDGFQLVAQRRCEDIDECQDPDTCSQLCVNLEGGYKCQCEEGFQLDPHTKACKAVGSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1113 bp, Artificial Sequence
  hVK3LP-hLDLR-EGFAB-(G4S)4-sssmthIgG nucleotide
aagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 20 - protein, 363 aa, Artificial Sequence
  hVK3LP-hLDLR-EGFAB-(G4S)4-sssmthIgG peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1041 bp, Artificial Sequence
  hVK3LP-hLDLR-EGFAB-H306Y-sssmthIgG nucleotide
aagcttgccaccatggaaaccccagcgcagcttctcttcctcctgctactctggctcccagataccaccggtggagggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 22 - protein, 339 aa, Artificial Sequence
  hVK3LP-hLDLR-EGFAB-H306Y-sssmthIgG peptide
METPAQLLFLLLLWLPDTTGGGTNECLDNNGGCSYVCNDLKIGYECLCPDGFQLVAQRRCEDIDECQDPDTCSQLCVNLEGGYKCQCEEGFQLDPHTKACKAVGSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - DNA, 1041 bp, Artificial Sequence
  hVK3LP-hLDLR-EGFAB-sssmthIgG nucleotide
aagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 24 - protein, 339 aa, Artificial Sequence; identical to SEQ ID NO: 22
  hVK3LP-hLDLR-EGFAB-sssmthIgG peptide

SEQ ID NO: 25 - DNA, 1665 bp, Artificial Sequence
  hVK3LP-hLDLR-EGFAB-H306Y-(G4S)4-sssmthIgG-NLG-hCD40 nucleotide
aagcttgccaccatggaaaccccagcgcagcttctcttcctcctgctactctggctcccagataccaccggtggagggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctctccggaggaggtggctcaggtggtggaggatctggaggaggtgggagtggtggaggtggttcgaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataa

SEQ ID NO: 26 - protein, 549 aa, Artificial Sequence
  hVK3LP-hLDLR-EGFAB-H306Y-(G4S)4-sssmthIgG-NLG-hCD40 peptide
METPAQLLFLLLLWLPDTTGGGTNECLDNNGGCSYVCNDLKIGYECLCPDGFQLVAQRRCEDIDECQDPDTCSQLCVNLEGGYKCQCEEGFQLDPHTKACKAVGSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 27 - DNA, 1593 bp, Artificial Sequence
  hVK3LP-hLDLR-EGFAB-H306Y-sssmthIgG-NLG-hCD40 nucleotide
aagcttgccaccatggaaaccccagcgcagcttctcttcctcctgctactctggctcccagataccaccggtggagggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataa

SEQ ID NO: 28 - protein, 525 aa, Artificial Sequence
  hVK3LP-hLDLR-EGFAB-H306Y-sssmthIgG-NLG-hCD40 peptide
METPAQLLFLLLLWLPDTTGGGTNECLDNNGGCSYVCNDLKIGYECLCPDGFQLVAQRRCEDIDECQDPDTCSQLCVNLEGGYKCQCEEGFQLDPHTKACKAVGSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 29 - DNA, 1665 bp, Artificial Sequence
  hVK3LP-hLDLR-EGFAB-(G4S)4-sssmthIgG-NLG-hCD40-4S nucleotide
aagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctctccggaggaggtggctcaggtggtggaggatctggaggaggtgggagtggtggaggtggttcgaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataa

SEQ ID NO: 30 - protein, 549 aa, Artificial Sequence
  hVK3LP-hLDLR-EGFAB-(G4S)4-sssmthIgG-NLG-hCD40-4S peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 31 - DNA, 1593 bp, Artificial Sequence
  hVK3LP-hLDLR-EGFAB-sssmthIgG-NLG-hCD40 nucleotide
aagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataa

SEQ ID NO: 32 - protein, 525 aa, Artificial Sequence
  hVK3LP-hLDLR-EGFAB-sssmthIgG-NLG-hCD40 peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 33 - DNA, 1665 bp, Artificial Sequence
  hVK3LP-hCD40-(G4S)4-sssmthIgG-NLG-LDLR-EGFAB-H306Y nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctaga

SEQ ID NO: 34 - protein, 547 aa, Artificial Sequence
  hVK3LP-hCD40-(G4S)4-sssmthIgG-NLG-LDLR-EGFAB-H306Y peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSYVCNDLKIGYECLCPDGFQLVAQRRCEDIDECQDPDTCSQLCVNLEGGYKCQCEEGFQLDPHTKACKAVGS

SEQ ID NO: 35 - DNA, 1665 bp, Artificial Sequence
  hVK3LP-hCD40-(G4S)4-sssmthIgG-NLG-LDLR-EGFAB WT nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctaga

SEQ ID NO: 36 - protein, 547 aa, Artificial Sequence
  hVK3LP-hCD40-(G4S)4-sssmthIgG-NLG-LDLR-EGFAB WT peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSHVCNDLKIGYECLCPDGFQLVAQRRCEDIDECQDPDTCSQLCVNLEGGYKCQCEEGFQLDPHTKACKAVGS

SEQ ID NO: 37 - DNA, 48 bp, Artificial Sequence
  Synthetic oligonucleotide
gttaagcttgccaccatggttcgtctgcctctgcagtgcgtcctctgg

SEQ ID NO: 38 - DNA, 48 bp, Artificial Sequence
  Synthetic oligonucleotide
tctagattatcactgtctctcctgcactgagatgcgactctctttgcc

SEQ ID NO: 39 - DNA, 834 bp, Homo sapiens
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtga

SEQ ID NO: 40 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 41 - DNA, 48 bp, Artificial Sequence
  Synthetic oligonucleotide
accggtgaaccacccactgcatgcagagaaaaacagtacctaataaac

SEQ ID NO: 42 - DNA, 41 bp, Artificial Sequence
  Synthetic oligonucleotide
ctcgagatctggctcgcagatggtatcagaaacccctgtag

SEQ ID NO: 43 - DNA, 44 bp, Artificial Sequence
  Synthetic oligonucleotide
ctcgagatctggaccacagacaacatcagtcttgtttgtgcctg

SEQ ID NO: 44 - DNA, 40 bp, Artificial Sequence
  Synthetic oligonucleotide
ctcgagatctgaatcctggggaccacagacaacatcagtc

SEQ ID NO: 45 - DNA, 32 bp, Artificial Sequence
  Synthetic oligonucleotide
gatatcgaaccacccactgcatgcagagaaaa

SEQ ID NO: 46 - DNA, 45 bp, Artificial Sequence
  Synthetic oligonucleotide
tctagattatcatggaccacagacaacatcagtcttgtttgtgcc

SEQ ID NO: 47 - DNA, 44 bp, Artificial Sequence
  Synthetic oligonucleotide
tctagattatcatggctcgcagatggtatcagaaacccctgtag

SEQ ID NO: 48 - DNA, 29 bp, Artificial Sequence
  Synthetic oligonucleotide
tctagattatcaatcctggggaccacaga

SEQ ID NO: 49 - DNA, 1362 bp, Artificial Sequence
  hCD40-4s-(G4S)4-SSSH-mthIgG-P238S-P331S nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 50 - protein, 446 aa, Artificial Sequence
  hCD40-4s-(G4S)4-SSSH-mthIgG-P238S-P331S peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - DNA, 1371 bp, Artificial Sequence
  hCD40-4L-(G4S)4-SSSH-mthIgG P238S-P331S nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggattcagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 52 - protein, 449 aa, Artificial Sequence
  hCD40-4L-(G4S)4-SSSH-mthIgG P238S-P331S peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 1233 bp, Artificial Sequence
  hCD40-3-(G4S)4-SSSH-mthIgGP238S-P331S nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 54 - protein, 403 aa, Artificial Sequence
  hCD40-3-(G4S)4-SSSH-mthIgGP238S-P331S peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 64 bp, Artificial Sequence
  Synthetic oligonucleotide
agatctcgtcgatggaggttcttcaaccacaagcccagtgaatgtgacatctccctctctcgag

SEQ ID NO: 56 - protein, 21 aa, Artificial Sequence
  Synthetic peptide
DLVDGGSSTTSPVNVTSPSLE

SEQ ID NO: 57 - DNA, 54 bp, Artificial Sequence
  Synthetic oligonucleotide
gtcgacggtgctagcagccctgtgaatgtgagcagccctagcgtgcaggatatc

SEQ ID NO: 58 - protein, 18 aa, Artificial Sequence
  Synthetic peptide
VDGASSPVNVSSPSVQDI

SEQ ID NO: 59 - DNA, 1914 bp, Artificial Sequence
  huVK3LP-hCD40-4s-(G4S)4-SSSH-mthIgG P238S-P331S-NLG-hCD40-4s nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataa

SEQ ID NO: 60 - protein, 632 aa, Artificial Sequence
  huVK3LP-hCD40-4s-(G4S)4-SSSH-mthIgG P238S-P331S-NLG-hCD40-4s peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 61 - DNA, 1278 bp, Artificial Sequence
  NL-hCD40-4s-nolker-SSSH mthIgG P238S- P331S nucleotide
atggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 62 - protein, 422 aa, Artificial Sequence
  NL-hCD40-4s-nolker-SSSH mthIgG P238S- P331S peptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1362 bp, Artificial Sequence
  NL-hCD40-4S-(G4S)4-SSSH-mthIgG P238S P331S nucleotide
aagcttgccaccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 64 - protein, 446 aa, Artificial Sequence
  NL-hCD40-4S-(G4S)4-SSSH-mthIgG P238S P331S peptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1344 bp, Artificial Sequence
  huVK3LP-hLDLR EGF AB WT-SSSH mthIgG-NLG-EGF AB WT nucleotide
aagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctaga

SEQ ID NO: 66 - protein, 440 aa, Artificial Sequence
  huVK3LP-hLDLR EGF AB WT-SSSH mthIgG-NLG-EGF AB WT peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSHVCNDLKIGYECLCPDGFQLVAQRRCEDIDECQDPDTCSQLCVNLEGGYKCQCEEGFQLDPHTKACKAVGS

SEQ ID NO: 67 - DNA, 1417 bp, Artificial Sequence
  huVK3LP-hLDLR EGF-AB H306Y-(G4S)4]SSSH mthIgG-NLG-hLDLR EGF-AB H306Y nucleotide
aagcttgccaccatggaaaccccagcgcagcttctcttcctcctgctactctggctcccagataccaccggtggagggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctctccggaggaggtggctcaggtggtggaggatctggaggaggtgggagtggtggaggtggttcgaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctagaa

SEQ ID NO: 68 - protein, 464 aa, Artificial Sequence
  huVK3LP-hLDLR EGF-AB H306Y-(G4S)4]SSSH mthIgG-NLG-hLDLR EGF-AB H306Y peptide
METPAQLLFLLLLWLPDTTGGGTNECLDNNGGCSYVCNDLKIGYECLCPDGFQLVAQRRCEDIDECQDPDTCSQLCVNLEGGYKCQCEEGFQLDPHTKACKAVGSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSYVCNDLKIGYECLCPDGFQLVAQRRCEDIDECQDPDTCSQLCVNLEGGYKCQCEEGFQLDPHTKACKAVGS

SEQ ID NO: 69 - DNA, 1416 bp, Artificial Sequence
  huVK3LP-hLDLR EGF AB WT-(G4S)4-SSSH mthIgG-NLG-EGF AB WT nucleotide
aagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctctccggaggaggtggctcaggtggtggaggatctggaggaggtgggagtggtggaggtggttcgaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctaga

SEQ ID NO: 70 - protein, 464 aa, Artificial Sequence
  huVK3LP-hLDLR EGF AB W-(G4S)4-SSSH mthIgG-NLG-EGF AB WT peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSHVCNDLKIGYECLCPDGFQLVAQRRCEDIDECQDPDTCSQLCVNLEGGYKCQCEEGFQLDPHTKACKAVGS

SEQ ID NO: 71 - DNA, 1345 bp, Artificial Sequence
  huVK3LP-hLDLR EGF AB H306Y-SSSH-mthIgG-NLG-EGF AB nucleotide
aagcttgccaccatggaaaccccagcgcagcttctcttcctcctgctactctggctcccagataccaccggtggagggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcctacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctagaa

SEQ ID NO: 72 - protein, 440 aa, Artificial Sequence
  huVK3LP-hLDLR EGF AB H306Y-SSSH-mthIgG-NLG-EGF AB peptide
METPAQLLFLLLLWLPDTTGGGTNECLDNNGGCSYVCNDLKIGYECLCPDGFQLVAQRRCEDIDECQDPDTCSQLCVNLEGGYKCQCEEGFQLDPHTKACKAVGSDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSYVCNDLKIGYECLCPDGFQLVAQRRCEDIDECQDPDTCSQLCVNLEGGYKCQCEEGFQLDPHTKACKAVGS

SEQ ID NO: 73 - DNA, 1362 bp, Artificial Sequence
  mCD40-4 (G4S)4 SSSH-mthIgG nucleotide
atggaaaccccagcgcagcttctcttcctcctgctactctggctcccagataccaccggtcagtgtgttacgtgcagtgacaaacagtacctccacgatggccagtgctgtgatttgtgccagccaggaagccgactgacaagccactgcacagctcttgagaagacccaatgccacccatgtgactcaggcgaattctcagcccagtggaacagggagattcgctgtcaccagcacagacactgtgaacccaatcaagggcttcgggttaagaaggagggcaccgcagaatcagacactgtctgtacctgtaaggaaggacaacactgcaccagcaaggattgcgaggcatgtgctcagcacacgccctgtatccctggctttggagttatggagatggccactgagaccactgataccgtctgtcatccctgcccagtcggcttcttctccaatcagtcatcacttttcgaaaagtgttatccctggacaagctgtgaggataagaacttggaggtcctacagaaaggaacgagtcagactaatgtcatctgtgaaaaggtggtcaagaaaccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 74 - protein, 450 aa, Artificial Sequence
  mCD40-4 (G4S)4 SSSH-mthIgG peptide
METPAQLLFLLLLWLPDTTGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICEKVVKKPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1233 bp, Artificial Sequence
  NL-mCD40-3-(G4S)4-SSSH-mthIgG nucleotide
aagcttgccaccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggtctgtgagagctgtgtcctgcaccgctcatgctcgcccggctttggggtcaagcagattgctacaggggtttctgataccatctgcgagccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 76 - protein, 403 aa, Artificial Sequence
  NL-mCD40-3-(G4S)4-SSSH-mthIgG peptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEVCESCVLHRSCSPGFGVKQIATGVSDTICEPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 1161 bp, Artificial Sequence
  NL-mCD40-3-no lnker-SSSH-mthIgG nucleotide
aagcttgccaccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggtctgtgagagctgtgtcctgcaccgctcatgctcgcccggctttggggtcaagcagattgctacaggggtttctgataccatctgcgagccagatctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 78 - protein, 379 aa, Artificial Sequence
  NL-mCD40-3-no lnker-SSSH-mthIgG peptide
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEVCESCVLHRSCSPGFGVKQIATGVSDTICEPDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1464 bp, Artificial Sequence
  huVK3LP-hCTLA4-(G4S)4-SSSH-mthIgG P238S P331S-NLG-hLDLR EGF-AB nucleotide
gttaagcttgccaccatggaaaccccagcgcagcttctcttcctcctgctactctggctcccagataccaccggtatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgaagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggtgctagcagccatgtgaatgtgagcagccctagcgtgcaggatgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctcatgataatctaga

SEQ ID NO: 80 - protein, 479 aa, Artificial Sequence
  huVK3LP-hCTLA4-(G4S)4-SSSH-mthIgG P238S P331S NLG-hLDLR EGF-AB peptide
METPAQLLFLLLLWLPDTTGMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSEDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSHVCNDLKIGYECLCPDGFQLVAQRRCEDIDECQDPDTCSQLCVNLEGGYKCQCEEGFQLDPHTKACKAVGS

SEQ ID NO: 81 - DNA, 1719 bp, Artificial Sequence
  huVK3LP-hCTLA4-no lnker-SSSH- mthIgG P238S P331S-NLG-hCD40-4S nucleotide
gttaagcttgccaccatggaaaccccagcgcagcttctcttcctcctgctactctggctcccagataccaccggtatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgaagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataatctaga

SEQ ID NO: 82 - protein, 564 aa, Artificial Sequence
  huVK3LP-hCTLA4-nolker-SSSH-mthIgG P238S P331S-NLG-hCD40-4S peptide
METPAQLLFLLLLWLPDTTGMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSEDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 83 - DNA, 1590 bp, Artificial Sequence
  huVK3LP-hCTLA4-(G4S)4-SSSH-mthIgG P238S P331S-NLG-hCD40-3 nucleotide
gttaagcttgccaccatggaaaccccagcgcagcttctcttcctcctgctactctggctcccagataccaccggtatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgaagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaagtcgacggagctagcagccccgtgaacgtgagcagccccagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgataatctaga

SEQ ID NO: 84 - protein, 521 aa, Artificial Sequence
  huVK3LP-hCTLA4-no lnker-SSSH-mthIgG P238S P331S-NLG-hCD40-3 peptide
METPAQLLFLLLLWLPDTTGMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSEDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSPVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEP

SEQ ID NO: 85 - DNA, 36 bp, Artificial Sequence
  Synthetic oligonucleotide
aagcttgccaccatggaaaccccagcgcagcttctc

SEQ ID NO: 86 - DNA, 27 bp, Artificial Sequence
  Synthetic oligonucleotide
ggagagatctggaccacagacaacatc

SEQ ID NO: 87 - DNA, 30 bp, Artificial Sequence
  Synthetic oligonucleotide
cagccaggacagacactggtgagtgactgc

SEQ ID NO: 88 - DNA, 30 bp, Artificial Sequence
  Synthetic oligonucleotide
gcagtcactcaccagtgtctgtcctggctg

SEQ ID NO: 89 - DNA, 30 bp, Artificial Sequence
  Synthetic oligonucleotide
cagccaggacagcacctggtgagtgactgc

SEQ ID NO: 90 - DNA, 30 bp, Artificial Sequence
  Synthetic oligonucleotide
gcagtcactcaccaggtgctgtcctggctg

SEQ ID NO: 91 - DNA, 39 bp, Artificial Sequence
  Synthetic oligonucleotide
tgccttccttgcggttcaagcgaattcctagacacctgg

SEQ ID NO: 92 - DNA, 39 bp, Artificial Sequence
  Synthetic oligonucleotide
ccaggtgtctaggaattcgcttgaaccgcaaggaaggca

SEQ ID NO: 93 - DNA, 39 bp, Artificial Sequence
  Synthetic oligonucleotide
tgccttccttgcggttacagcgaattcctagacacctgg

SEQ ID NO: 94 - DNA, 39 bp, Artificial Sequence
  Synthetic oligonucleotide
ccaggtgtctaggaattcgctgtaaccgcaaggaaggca

SEQ ID NO: 95 - DNA, 39 bp, Artificial Sequence
  Synthetic oligonucleotide
tgccttccttgcggtgaaagcacattcctagacacctgg

SEQ ID NO: 96 - DNA, 39 bp, Artificial Sequence
  Synthetic oligonucleotide
ccaggtgtctaggaatgtgctttcaccgcaaggaaggca

SEQ ID NO: 97 - DNA, 39 bp, Artificial Sequence
  Synthetic oligonucleotide
tgccttccttgcggttacagcacattcctagacacctgg

SEQ ID NO: 98 - DNA, 39 bp, Artificial Sequence
  Synthetic oligonucleotide
ccaggtgtctaggaatgtgctgtaaccgcaaggaaggca

SEQ ID NO: 99 - DNA, 39 bp, Artificial Sequence
  Synthetic oligonucleotide
agcgaattcctacagacctggaacagagagacacactgc

SEQ ID NO: 100 - DNA, 39 bp, Artificial Sequence
  Synthetic oligonucleotide
gcagtgtgtctctctgttccaggtctgtaggaattcgct

SEQ ID NO: 101 - DNA, 33 bp, Artificial Sequence
  Synthetic oligonucleotide
gacacctggaacagaacaacacactgccaccag

SEQ ID NO: 102 - DNA, 33 bp, Artificial Sequence
  Synthetic oligonucleotide
ctggtggcagtgtgttgttctgttccaggtgtc

SEQ ID NO: 103 - DNA, 44 bp, Artificial Sequence
  Synthetic oligonucleotide
gacacctggaacagagagacacagtgccaccagcacaaatactg

SEQ ID NO: 104 - DNA, 44 bp, Artificial Sequence
  Synthetic oligonucleotide
cagtatttgtgctggtggcactgtgtctctctgttccaggtgtc

SEQ ID NO: 105 - DNA, 34 bp, Artificial Sequence
  Synthetic oligonucleotide
gagacacactgccaccagcaccactactgcgacc

SEQ ID NO: 106 - DNA, 34 bp, Artificial Sequence
  Synthetic oligonucleotide
ggtcgcagtagtggtgctggtggcagtgtgtctc

SEQ ID NO: 107 - DNA, 34 bp, Artificial Sequence
  Synthetic oligonucleotide
gagacacactgccaccagcactcctactgcgacc

SEQ ID NO: 108 - DNA, 34 bp, Artificial Sequence
  Synthetic oligonucleotide
ggtcgcagtaggagtgctggtggcagtgtgtctc

SEQ ID NO: 109 - DNA, 34 bp, Artificial Sequence
  Synthetic oligonucleotide
gagacacactgccaccagcacacatactgcgacc

SEQ ID NO: 110 - DNA, 34 bp, Artificial Sequence
  Synthetic oligonucleotide
ggtcgcagtatgtgtgctggtggcagtgtgtctc

SEQ ID NO: 111 - DNA, 30 bp, Artificial Sequence
  Synthetic oligonucleotide
ccagcacaaatactgcgactacaacctagg

SEQ ID NO: 112 - DNA, 30 bp, Artificial Sequence
  Synthetic oligonucleotide
cctaggttgtagtcgcagtatttgtgctgg

SEQ ID NO: 113 - DNA, 37 bp, Artificial Sequence
  Synthetic oligonucleotide
acaaatactgcgaccccaccctagggcttcgggtcca

SEQ ID NO: 114 - DNA, 37 bp, Artificial Sequence
  Synthetic oligonucleotide
tggacccgaagccctagggtggggtcgcagtatttgt

SEQ ID NO: 115 - DNA, 32 bp, Artificial Sequence
  Synthetic oligonucleotide
ggcttcgggtccagtcaaagggcacctcagaa

SEQ ID NO: 116 - DNA, 32 bp, Artificial Sequence
  Synthetic oligonucleotide
ttctgaggtgccctttgactggacccgaagcc

SEQ ID NO: 117 - DNA, 33 bp, Artificial Sequence
  Synthetic oligonucleotide
gaaggctggcactgttacagtgaggtctgtgag

SEQ ID NO: 118 - DNA, 33 bp, Artificial Sequence
  Synthetic oligonucleotide
ctcacagacctcactgtaacagtgccagccttc

SEQ ID NO: 119 - DNA, 33 bp, Artificial Sequence
  Synthetic oligonucleotide
tggcactgtacgagtcgggtctgtgagagctgt

SEQ ID NO: 120 - DNA, 33 bp, Artificial Sequence
  Synthetic oligonucleotide
acagctctcacagacccgactcgtacagtgcca

SEQ ID NO: 121 - DNA, 43 bp, Artificial Sequence
  Synthetic oligonucleotide
tggcactgtacgagtcgggtctgtcagagctgtgtcctgcacc

SEQ ID NO: 122 - DNA, 43 bp, Artificial Sequence
  Synthetic oligonucleotide
ggtgcaggacacagctctgacagacccgactcgtacagtgcca

SEQ ID NO: 123 - DNA, 43 bp, Artificial Sequence
  Synthetic oligonucleotide
tggcactgtacgagtgaggtctgtcagagctgtgtcctgcacc

SEQ ID NO: 124 - DNA, 43 bp, Artificial Sequence
  Synthetic oligonucleotide
ggtgcaggacacagctctgacagacctcactcgtacagtgcca

SEQ ID NO: 125 - DNA, 33 bp, Artificial Sequence
  Synthetic oligonucleotide
gaaggctggcactgttccagtgaggtctgtgag

SEQ ID NO: 126 - DNA, 33 bp, Artificial Sequence
  Synthetic oligonucleotide
ctcacagacctcactggaacagtgccagccttc

SEQ ID NO: 127 - DNA, 37 bp, Artificial Sequence
  Synthetic oligonucleotide
acaaatactgcgacccccaactagggcttcgggtcca

SEQ ID NO: 128 - DNA, 37 bp, Artificial Sequence
  Synthetic oligonucleotide
tggacccgaagccctagttgggggtcgcagtatttgt

SEQ ID NO: 129 - DNA, 43 bp, Artificial Sequence
  Synthetic oligonucleotide
tggcactgtacgagtaacgtctgtcagagctgtgtcctgcacc

SEQ ID NO: 130 - DNA, 43 bp, Artificial Sequence
  Synthetic oligonucleotide
ggtgcaggacacagctctgacagacgttactcgtacagtgcca

SEQ ID NO: 131 - DNA, 32 bp, Artificial Sequence
  Synthetic oligonucleotide
caccagcacaaatactgcgactggaacctagg

SEQ ID NO: 132 - DNA, 32 bp, Artificial Sequence
  Synthetic oligonucleotide
cctaggttccagtcgcagtatttgtgctggtg

SEQ ID NO: 133 - DNA, 582 bp, Artificial Sequence
  Synthetic oligonucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggttgatctaga

SEQ ID NO: 134 - protein, 187 aa, Artificial Sequence
  Synthetic peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCG

SEQ ID NO: 135 - DNA, 1167 bp, Artificial Sequence
  huVK3LP-hCD40-4-(G4S)4-hCD40-STOP nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctgatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataatctaga

SEQ ID NO: 136 - protein, 381 aa, Artificial Sequence
  huVK3LP-hCD40-4-(G4S)4-hCD40-STOP
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 137 - DNA, 1200 bp, Artificial Sequence
  huLDLR-EGF-AB-Bprop-EGF-C modules nucleotide
gggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcaca

SEQ ID NO: 138 - protein, 400 aa, Artificial Sequence
  huLDLR-EGFAB-Bprop-EGF-C modules peptide
GTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLT

SEQ ID NO: 139 - DNA, 1218 bp, Artificial Sequence
  huLDLR-EGFAB-Bprop-EGFC-5 nucleotide
accggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagatctcgag

SEQ ID NO: 140 - protein, 406 aa, Artificial Sequence
  huLDLR-EGFAB-Bprop-EGFC-5 peptide
TGGGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTDLE

SEQ ID NO: 141 - DNA, 1215 bp, Artificial Sequence
  huLDLR-EGFAB-Bprop-EGFC-3 nucleotide
cccgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacatgataatctaga

SEQ ID NO: 142 - protein, 401 aa, Artificial Sequence
  huLDLR-EGFAB-Bprop-EGF-C 3 peptide
PGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLT

SEQ ID NO: 143 - DNA, 1053 bp, Artificial Sequence
  huLDLR-EGFAB-Bprop-5 nucleotide
accggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctagatctcgag

SEQ ID NO: 144 - protein, 351 aa, Artificial Sequence
  huLDLR-EGFAB-Bprop-5 peptide
TGGGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLDLE

SEQ ID NO: 145 - DNA, 1047 bp, Artificial Sequence
  huLDLR-EGFAB-Bprop-3 nucleotide
cccgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaactgataatctaga

SEQ ID NO: 146 - protein, 345 aa, Artificial Sequence
  huLDLR-EGFAB-Bprop-3 peptide
PGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHN

SEQ ID NO: 147 - DNA, 2445 bp, Artificial Sequence
  huCD40-(G4S)4-SSSH-mthIgG-NLG-huLDLR-EGFAB-Bprop nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaactgataatctaga

SEQ ID NO: 148 - protein, 807 aa, Artificial Sequence
  huCD40-(G4S)4-SSSH-mthIgG-NLG-huLDLR-EGFAB-Bprop peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHN

SEQ ID NO: 149 - DNA, 2601 bp, Artificial Sequence
  huCD40-(G4S)4-SSSH-mthIgG-NLG-huLDLR-EGFAB-Bprop-EGFC nucleotide
atggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacatgataatctaga

SEQ ID NO: 150 - protein, 863 aa, Artificial Sequence
  huCD40-(G4S)4-SSSH-mthIgG-NLG-huLDLR-EGFAB-Bprop-EGFC peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLT

SEQ ID NO: 151 - DNA, 2124 bp, Artificial Sequence
  huCD40-(G4S)4-SSSH-mthIgG-NLG-anti-hPCSK9scFv nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccaggtggcggtggctcgggcggtggtgggtcgggtggcggcggatcgctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgatattcagatgacccagagcccgagcagcctgagcgcgagcgtgggcgatcgcgtgaccattacctgccgcgcgagccagggcattagcagcgcgctggcgtggtatcagcagaaaccgggcaaagcgccgaaactgctgatttatagcgcgagctatcgctataccggcgtgccgagccgctttagcggcagcggcagcggcaccgattttacctttaccattagcagcctgcagccggaagatattgcgacctattattgccagcagcgctatagcctgtggcgcacctttggccagggcaccaaactggaaattaaacgcggcggcggcggcagcggcggcggcggcagcggcggcggcggcagcggcggcggcggcagccaggtgcagctggtgcagagcggcgcggaagtgaaaaaaccgggcgcgagcgtgaaagtgagctgcaaagcgagcggctatacctttaccagctattatatgcattgggtgcgccaggcgccgggccagggcctggaatggatgggcgaaattagcccgtttggcggccgcaccaactataacgaaaaatttaaaagccgcgtgaccatgacccgcgataccagcaccagcaccgtgtatatggaactgagcagcctgcgcagcgaagataccgcggtgtattattgcgcgcgcgaacgcccgctgtatgcgagcgatctgtggggccagggcaccaccgtgaccgtgagcagctgataatctaga

SEQ ID NO: 152 - protein, 700 aa, Artificial Sequence
  huCD40-(G4S)4-SSSH-mthIgG-NLG-anti-hPCSK9scFv peptide
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPGGGGSGGGGSGGGGSLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIDIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRGGGGSGGGGSGGGGSGGGGSQVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSS

SEQ ID NO: 153 - DNA, 2079 bp, Artificial Sequence
  Anti-hPCSK9 scFv-SSSH-mthIgG-NLG-hCD40-4s nucleotide
aagcttgccaccatggaaaccccagcgcagcttctcttcctcctgctactctggctcccagataccaccggtgatattcagatgacccagagcccgagcagcctgagcgcgagcgtgggcgatcgcgtgaccattacctgccgcgcgagccagggcattagcagcgcgctggcgtggtatcagcagaaaccgggcaaagcgccgaaactgctgatttatagcgcgagctatcgctataccggcgtgccgagccgctttagcggcagcggcagcggcaccgattttacctttaccattagcagcctgcagccggaagatattgcgacctattattgccagcagcgctatagcctgtggcgcacctttggccagggcaccaaactggaaattaaacgcggcggcggcggcagcggcggcggcggcagcggcggcggcggcagcggcggcggcggcagccaggtgcagctggtgcagagcggcgcggaagtgaaaaaaccgggcgcgagcgtgaaagtgagctgcaaagcgagcggctatacctttaccagctattatatgcattgggtgcgccaggcgccgggccagggcctggaatggatgggcgaaattagcccgtttggcggccgcaccaactataacgaaaaatttaaaagccgcgtgaccatgacccgcgataccagcaccagcaccgtgtatatggaactgagcagcctgcgcagcgaagataccgcggtgtattattgcgcgcgcgaacgcccgctgtatgcgagcgatctgtggggccagggcaccaccgtgaccgtgagcagcctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctgtctccgggtaaagtcgacggtgctagcagccatgtgaatgtgagcagccctagcgtgcaggatatcgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccatgataatctaga

SEQ ID NO: 154 - protein, 685 aa, Artificial Sequence
  Anti-hPCSK9 scFv-SSSH- mthIgG-NLG- hCD40-4s peptide
METPAQLLFLLLLWLPDTTGDIQMTQSPSSLSASVGDRVTITCRASQGISSALAWYQQKPGKAPKLLIYSASYRYTGVPSRFSGSGSGTDFTFTISSLQPEDIATYYCQQRYSLWRTFGQGTKLEIKRGGGGSGGGGSGGGGSGGGGSQVQLVQSGAEVKKPGASVKVSCKASGYTFTSYYMHWVRQAPGQGLEWMGEISPFGGRTNYNEKFKSRVTMTRDTSTSTVYMELSSLRSEDTAVYYCARERPLYASDLWGQGTTVTVSSLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKVDGASSHVNVSSPSVQDIEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 155 - protein, 4 aa, Artificial Sequence
  Synthetic peptide
VGDR

SEQ ID NO: 156 - protein, 4 aa, Artificial Sequence
  Synthetic peptide
LSVT

SEQ ID NO: 157 - protein, 4 aa, Artificial Sequence
  Synthetic peptide
PPKT

SEQ ID NO: 158 - protein, 4 aa, Artificial Sequence
  Synthetic peptide
PVLT

SEQ ID NO: 159 - protein, 4 aa, Artificial Sequence
  Synthetic peptide
AVAT

SEQ ID NO: 160 - protein, 5 aa, Artificial Sequence
  Synthetic peptide
VNVTL

SEQ ID NO: 161 - protein, 3 aa, Artificial Sequence
  Synthetic peptide
PPQ

SEQ ID NO: 162 - protein, 6 aa, Artificial Sequence
  Synthetic peptide
HQHPPG

SEQ ID NO: 163 - protein, 12 aa, Artificial Sequence
  Synthetic peptide
RGLYVFQGDSSP

SEQ ID NO: 164 - protein, 7 aa, Artificial Sequence
  Synthetic peptide
KAVGSIA

SEQ ID NO: 165 - protein, 8 aa, Artificial Sequence
  Synthetic peptide
HNLTQPRG

SEQ ID NO: 166 - protein, 6 aa, Artificial Sequence
  Synthetic peptide
QGDSSP

SEQ ID NO: 167 - DNA, 1989 bp, Artificial Sequence
  huVK3LP-hLDLR-EGFAB-Bprop- EGFC-SSSH- mthIgG1 nucleotide
gttaagcttgccaccatggaaaccccagcgcagcttctcttcctcctgctactctggctcccagataccaccggtgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 168 - protein, 654 aa, Artificial Sequence
  huVK3LP-huLDLR-EGFAB- Bprop-EGFC-SSSH-mthIgG1 peptide
METPAQLLFLLLLWLPDTTGGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - DNA, 1812 bp, Artificial Sequence
  huVK3LP-hLDLR-EGFAB-Bprop-SSSH-mthIgG1 nucleotide
atggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctagatctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 170 - protein, 600 aa, Artificial Sequence
  huVK3LP-hLDLR-EGFAB-Bprop-SSSH-mthIgG1 peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLDLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - DNA, 1899 bp, Artificial Sequence
  huVK3LP-hLDLR-EGFAB-Bprop-(G4S)4-SSSH-mthIgG1 nucleotide
gttaagcttgccaccatggaaaccccagcgcagcttctcttcctcctgctactctggctcccagataccaccggtggagggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctagatctctccggaggaggtggctcaggtggtggaggatctggaggaggtgggagtggtggaggtggttctaccggtctcgagcccaaatcttctgacaaaactcacacatctccaccgtccccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 172 - protein, 624 aa, Artificial Sequence
  huVK3LP-hLDLR-EGFAB-Bprop-(G4S)4-SSSH-mthIgG1 peptide
METPAQLLFLLLLWLPDTTGGGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - DNA, 2061 bp, Artificial Sequence
  huVK3LP-hLDLR-EGFAB-Bprop-EGFC-(G4S)4-SSSH-mthIgG1 nucleotide
aagcttgccaccatggaaaccccagcgcagcttctcttcctcctgctactctggctcccagataccaccggtgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcaactcagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 174 - protein, 679 aa, Artificial Sequence
  huVK3LP-hLDLR-EGFAB-Bprop- EGFC-(G4S)4- SSSH-mthIgG1 peptide
METPAQLLFLLLLWLPDTTGGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLNSDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 175 - DNA, 33 bp, Artificial Sequence
  Synthetic oligonucleotide
tggcactgtacgagtgaggcctgtgagagctgt

SEQ ID NO: 176 - DNA, 33 bp, Artificial Sequence
  Synthetic oligonucleotide
acagctctcacaggcctcactcgtacagtgcca

SEQ ID NO: 177 - DNA, 43 bp, Artificial Sequence
  Synthetic oligonucleotide
tggcactgtacgagtgaggcctgtgagagctgtgtcctgcacc

SEQ ID NO: 178 - DNA, 41 bp, Artificial Sequence
  Synthetic oligonucleotide
tgcaggacacagctctcacaggcctcactcgtacagtgcca

SEQ ID NO: 179 - DNA, 41 bp, Artificial Sequence
  Synthetic oligonucleotide
gcactgtacgagtgaggcctgtcagagctgtgtcctgcacc

SEQ ID NO: 180 - DNA, 43 bp, Artificial Sequence
  Synthetic oligonucleotide
ggtgcaggacacagctctgacaggcctcactcgtacagtgcca

SEQ ID NO: 181 - DNA, 43 bp, Artificial Sequence
  Synthetic oligonucleotide
tggcactgtacgagtaacgcctgtcagagctgtgtcctgcacc

SEQ ID NO: 182 - DNA, 43 bp, Artificial Sequence
  Synthetic oligonucleotide
ggtgcaggacacagctctgacaggcgttactcgtacagtgcca

SEQ ID NO: 183 - DNA, 33 bp, Artificial Sequence
  Synthetic oligonucleotide
tggcactgtacgagtcgggcctgtgagagctgt

SEQ ID NO: 184 - DNA, 33 bp, Artificial Sequence
  Synthetic oligonucleotide
acagctctcacaggcccgactcgtacagtgcca

SEQ ID NO: 185 - DNA, 43 bp, Artificial Sequence
  Synthetic oligonucleotide
tggcactgtacgagtcgggtctgccagagctgtgtcctgcacc

SEQ ID NO: 186 - DNA, 40 bp, Artificial Sequence
  Synthetic oligonucleotide
gcaggacacagctctggcagacccgactcgtacagtgcca

SEQ ID NO: 187 - DNA, 33 bp, Artificial Sequence
  Synthetic oligonucleotide
gaaggctggcactgttccagtgaggcctgtgag

SEQ ID NO: 188 - DNA, 33 bp, Artificial Sequence
  Synthetic oligonucleotide
ctcacaggcctcactggaacagtgccagccttc

SEQ ID NO: 189 - DNA, 1362 bp, Artificial Sequence
  hVK3LP-hCD40-4s-K46T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagacactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 190 - protein, 446 aa, Artificial Sequence
  hVK3LP-hCD40-4s-K46T-(g4s)4-sssH-mthIgG1 amino acid
METPAQLLFLLLLWLPDTTGEPPTACREKQYLINSQCCSLCQPGQTLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - DNA, 1362 bp, Artificial Sequence
  hVK3LP-hCD40-4s-K46H-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagcacctggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 192 - protein, 446 aa, Artificial Sequence
  hVK3LP-hCD40-4s-K46H-(g4s)4-sssH-mthIgG1 amino acid
METPAQLLFLLLLWLPDTTGEPPTACREKQYLINSQCCSLCQPGQHLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - DNA, 1362 bp, Artificial Sequence
  hVK3LP-hCD40-4s-E64S-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttcaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 194 - protein, 446 aa, Artificial Sequence
  hVK3LP-hCD40-4s-E64S-(g4s)4-sssH-mthIgG1 amino acid
METPAQLLFLLLLWLPDTTGEPPTACREKQYLINSQCCSLCQPGQKLVSDCTEFTETECLPCGSSEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - DNA, 1362 bp, Artificial Sequence
  hVK3LP-hCD40-4s-E64Y-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 196 - protein, 446 aa, Artificial Sequence
  hVK3LP-hCD40-4s-E64Y-(g4s)4-sssH-mthIgG1 amino acid
METPAQLLFLLLLWLPDTTGEPPTACREKQYLINSQCCSLCQPGQKLVSDCTEFTETECLPCGYSEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - DNA, 1362 bp, Artificial Sequence
  hVK3LP-hCD40-4s-E66T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcacattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 198 - protein, 446 aa, Artificial Sequence
  hVK3LP-hCD40-4s-E66T-(g4s)4-sssH-mthIgG1 amino acid
METPAQLLFLLLLWLPDTTGEPPTACREKQYLINSQCCSLCQPGQKLVSDCTEFTETECLPCGESTFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - DNA, 1362 bp, Artificial Sequence
  hVK3LP-hCD40-4s-D69Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctacagacctggaacagagagacacactgccaccagcacaaatactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 200 - protein, 446 aa, Artificial Sequence
  hVK3LP-hCD40-4s-D69Q-(g4s)4-sssH-mthIgG1 amino acid
METPAQLLFLLLLWLPDTTGEPPTACREKQYLINSQCCSLCQPGQKLVSDCTEFTETECLPCGESEFLQTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - DNA, 1362 bp, Artificial Sequence
  hVK3LP-hCD40-4s-E74T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagaacaacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 202 - protein, 446 aa, Artificial Sequence
  hVK3LP-hCD40-4s-E74T-(g4s)4-sssH-mthIgG1 amino acid
METPAQLLFLLLLWLPDTTGEPPTACREKQYLINSQCCSLCQPGQKLVSDCTEFTETECLPCGESEFLDTWNRTTHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - DNA, 1362 bp, Artificial Sequence
  hVK3LP-hCD40-4s-H76Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacagtgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 204 - protein, 446 aa, Artificial Sequence
  hVK3LP-hCD40-4s-H76Q-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETQCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - DNA, 1362 bp, Artificial Sequence
  hVK3LP-hCD40-4s-K81H-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcaccactactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 206 - protein, 446 aa, Artificial Sequence
  hVK3LP-hCD40-4s-K81H-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHHYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - DNA, 1362 bp, Artificial Sequence
  hVK3LP-hCD40-4s-K81S-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcactcctactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 208 - protein, 446 aa, Artificial Sequence
  hVK3LP-hCD40-4s-K81S-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHSYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - DNA, 1362 bp, Artificial Sequence
  hVK3LP-hCD40-4s-K81T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacacatactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 210 - protein, 446 aa, Artificial Sequence
  hVK3LP-hCD40-4s-K81T-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHTYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - DNA, 1362 bp, Artificial Sequence
  hVK3LP-hCD40-4s-P85W-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgactggaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 212 - protein, 446 aa, Artificial Sequence
  hVK3LP-hCD40-4s-P85W-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDWNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - DNA, 1362 bp, Artificial Sequence
  hVK3LP-hCD40-4s-P85Y-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgactacaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 214 - protein, 446 aa, Artificial Sequence
  hVK3LP-hCD40-4s-P85Y-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDYNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - DNA, 1362 bp, Artificial Sequence
  hVK3LP-hCD40-4s-N86Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgacccccaactagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 216 - protein, 446 aa, Artificial Sequence
  hVK3LP-hCD40-4s-N86Q-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPQLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - DNA, 1362 bp, Artificial Sequence
  hVK3LP-hCD40-4s-N86T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaccctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 218 - protein, 446 aa, Artificial Sequence
  hVK3LP-hCD40-4s-N86T-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPTLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - DNA, 1362 bp, Artificial Sequence
  hVK3LP-hCD40-4s-Q93S-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagtcaaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 220 - protein, 446 aa, Artificial Sequence
  hVK3LP-hCD40-4s-Q93S-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQSKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - DNA, 1362 bp, Artificial Sequence
  hVK3LP-hCD40-4s-T112S-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgttccagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 222 - protein, 446 aa, Artificial Sequence
  hVK3LP-hCD40-4s-T112S-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCSSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - DNA, 1362 bp, Artificial Sequence
  hVK3LP-hCD40-4s-T112Y-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgttacagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 224 - protein, 446 aa, Artificial Sequence
  hVK3LP-hCD40-4s-T112Y-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCYSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - DNA, 1362 bp, Artificial Sequence
  hVK3LP-hCD40-4s-E114R-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtcgggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 226 - protein, 446 aa, Artificial Sequence
  hVK3LP-hCD40-4s-E114R-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSRACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - DNA, 1362 bp, Artificial Sequence
  hVK3LP-hCD40-4s-E114N-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtaacgcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 228 - protein, 446 aa, Artificial Sequence
  hVK3LP-hCD40-4s-E114N-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSNACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - DNA, 1362 bp, Artificial Sequence
  hVK3LP-hCD40-4s-E117Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcttgtcagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 230 - protein, 446 aa, Artificial Sequence
  hVK3LP-hCD40-4s-E117Q-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSEACQSCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - DNA, 1362 bp, Artificial Sequence
  hVK3LP-hCD40-4s-E114N-E117Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtaacgcctgtcagagctgtgtcctgcaccgttcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 232 - protein, 446 aa, Artificial Sequence
  hVK3LP-hCD40-4s-E114N-E117Q-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSNACQSCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - DNA, 1362 bp, Artificial Sequence
  hVK3LP-hCD40-4s-E114R-E117Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtcgggcctgtcagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 234 - protein, 446 aa, Artificial Sequence
  hVK3LP-hCD40-4s-E114R-E117Q-(g4s)4-sssH-mthIgG1
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSRACQSCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - DNA, 1362 bp, Artificial Sequence
  hVK3LP-hCD40-4s-T112S-A115V-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgttccagtgaggtctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 236 - protein, 446 aa, Artificial Sequence
  hVK3LP-hCD40-4s-T112S-A115V-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCSSEVCESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - DNA, 1362 bp, Artificial Sequence
  hVK3LP-hCD40-4s-E114R-A115V-E117Q-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtcgggtctgtcagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 238 - protein, 446 aa, Artificial Sequence
  hVK3LP-hCD40-4s-E114R-A115V-E117Q-(g4s)4-sssH-mthIgG1 amino acid
METPAQLLFLLLLWLPDTTGEPPTACREKQYLINSQCCSLCQPGQKLVSDCTEFTETECLPCGESEFLDTWNRETHCHQHKYCDPNLGLRVQQKGTSETDTICTCEEGWHCTSRVCQSCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - DNA, 1362 bp, Artificial Sequence
  hVK3LP-hCD40-4s-E64Y-E66T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcacattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcttgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 240 - protein, 446 aa, Artificial Sequence
  hVK3LP-hCD40-4s-E64Y-E66T-(g4s)4-sssH-mthIgG1 amino acid
METPAQLLFLLLLWLPDTTGEPPTACREKQYLINSQCCSLCQPGQKLVSDCTEFTETECLPCGYSTFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - DNA, 1362 bp, Artificial Sequence
  hVK3LP-hCD40-4s-E64S-K81T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttcaagcgaattcctagacacctggaacagagagacacactgccaccagcacacatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 242 - protein, 446 aa, Artificial Sequence
  hVK3LP-hCD40-4s-E64S-K81T-(g4s)4-sssH-mthIgG1 amino acid
METPAQLLFLLLLWLPDTTGEPPTACREKQYLINSQCCSLCQPGQKLVSDCTEFTETECLPCGSSEFLDTWNRETHCHQHTYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - DNA, 1362 bp, Artificial Sequence
  hVK3LP-hCD40-4s-E64S-K81S-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttcaagcgaattcctagacacctggaacagagagacacactgccaccagcactcctactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 244 - protein, 446 aa, Artificial Sequence
  hVK3LP-hCD40-4s-E64S-K81S-(g4s)4-sssH-mthIgG1 amino acid
METPAQLLFLLLLWLPDTTGEPPTACREKQYLINSQCCSLCQPGQKLVSDCTEFTETECLPCGSSEFLDTWNRETHCHQHSYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - DNA, 1362 bp, Artificial Sequence
  hVK3LP-hCD40-4s-E64Y-K81T-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcacacatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 246 - protein, 446 aa, Artificial Sequence
  hVK3LP-hCD40-4s-E64Y-K81T-(g4s)4-sssH-mthIgG1 amino acid
METPAQLLFLLLLWLPDTTGEPPTACREKQYLINSQCCSLCQPGQKLVSDCTEFTETECLPCGYSEFLDTWNRETHCHQHTYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - DNA, 1362 bp, Artificial Sequence
  hVK3LP-hCD40-4s-E64Y-K81T-P85Y-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcacacatactgcgactacaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 248 - protein, 446 aa, Artificial Sequence
  hVK3LP-hCD40-4s-E64Y-K81T-P85Y-(g4s)4-sssH-mthIgG1 amino acid
METPAQLLFLLLLWLPDTTGEPPTACREKQYLINSQCCSLCQPGQKLVSDCTEFTETECLPCGYSEFLDTWNRETHCHQHTYCDYNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - DNA, 1362 bp, Artificial Sequence
  hVK3LP-hCD40-4s-E64S-K81H-L121P-(g4s)4-sssH-mthIgG1 nucleotide
aagcttgccaccatggaaaccccagcgcagcttctcttcctcctgctactctggctcccaggtaccaccggtgaaccacccactgcatgcagagaaaaacagtacctaataaacagtcagtgctgttctttgtgccagccaggacagaaactggtgagtgactgcacagagttcactgaaacggaatgccttccttgcggttcaagcgaattcctagacacctggaacagagagacacactgccaccagcaccactactgcgaccccaacctagggcttcgggtccagcagaagggcacctcagaaacagacaccatctgcacctgtgaagaaggctggcactgtacgagtgaggcctgtgagagctgtgtcccgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 250 - protein, 446 aa, Artificial Sequence
  hVK3LP-hCD40-4s-E64S-K81H-L121P-(g4s)4-sssH-mthIgG1 amino acid
METPAQLLFLLLLWLPGTTGEPPTACREKQYLINSQCCSLCQPGQKLVSDCTEFTETECLPCGSSEFLDTWNRETHCHQHHYCDPNLGLRVQQKGTSETDTICTCEEGWHCTSEACESCVPHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - DNA, 1363 bp, Artificial Sequence
  hVK3LP-hCD40-4s-E64Y-K81H-L121P-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcaccactactgcgaccccaacctagggcttcgggtccagcagaagggcacctcagaaacagacaccatctgcacctgtgaagaaggctggcactgtacgagtgaggcctgtgagagctgtgtcccgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctagaa

SEQ ID NO: 252 - protein, 446 aa, Artificial Sequence
  hVK3LP-hCD40-4s-E64Y-K81H-L121P-(g4s)4-sssH-mthIgG1 amino acid
METPAQLLFLLLLWLPDTTGEPPTACREKQYLINSQCCSLCQPGQKLVSDCTEFTETECLPCGYSEFLDTWNRETHCHQHHYCDPNLGLRVQQKGTSETDTICTCEEGWHCTSEACESCVPHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 253 - DNA, 1362 bp, Artificial Sequence
  hVK3LP-hCD40-4s-E64Y-K81H-P85Y-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcaccactactgcgactacaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 254 - protein, 446 aa, Artificial Sequence
  Synthetic peptide
METPAQLLFLLLLWLPDTTGEPPTACREKQYLINSQCCSLCQPGQKLVSDCTEFTETECLPCGYSEFLDTWNRETHCHQHHYCDYNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - DNA, 1362 bp, Artificial Sequence
  hVK3LP-hCD40-4s-E64Y-K81H-P85Y-L121P-(g4s)4-sssH-mthIgG1 nucleotide
aagcttgccaccatggaaaccccagcgcagcttctcttcctcctgctactctggctcccagataccaccggtgaaccacccactgcatgcagagaaaaacagtacctaataaacagtcagtgctgttctttgtgccagccaggacagaaactggtgagtgactgcacagagttcactgaaacggaatgccttccttgcggttacagcgaattcctagacacctggaacagagagacacactgccaccagcaccactactgcgactacaacctagggcttcgggtccagcagaagggcacctcagaaacagacaccatctgcacctgtgaagaaggctggcactgtacgagtgaggcctgtgagagctgtgtcccgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccagatctctccggaggaggtggctcaggtggtggaggatctggaggaggtgggagtggtggaggtggttctaccggtctcgagcccaaatcttctgacaaaactcacacatctccaccgagcccagcacctgaactcctgggaggatcgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctctctctctccgggtaaatgataatctaga

SEQ ID NO: 256 - protein, 446 aa, Artificial Sequence
  hVK3LP-hCD40-4s-E64Y-K81H-P85Y-L121P-(g4s)4-sssH-mthIgG1 amino acid
METPAQLLFLLLLWLPDTTGEPPTACREKQYLINSQCCSLCQPGQKLVSDCTEFTETECLPCGYSEFLDTWNRETHCHQHHYCDYNLGLRVQQKGTSETDTICTCEEGWHCTSEACESCVPHRSCSPGFGVKQIATGVSDTICEPCPVGFFSNVSSAFEKCHPWTSCETKDLVVQQAGTNKTDVVCGPDLSGGGGSGGGGSGGGGSGGGGSTGLEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 124 aa, Artificial Sequence
  translation of human CD40 domain 3 (CD40-3)
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICE

SEQ ID NO: 258 - protein, 166 aa, Artificial Sequence
  translation of human CD40 domain 4 (CD40-4S)
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVC

SEQ ID NO: 259 - protein, 168 aa, Artificial Sequence
  translation of human CD40 domain 4 (CD40-4L)
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGP

SEQ ID NO: 260 - protein, 257 aa, Artificial Sequence
  translation part of human CD40 variant 1
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 261 - protein, 239 aa, Artificial Sequence
  translation of mouse CD40 variant 5
QCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICEKVVKKPKDNEILPPAARRQDPQEMEDYPGHNTAAPVQETLHGCQPVTQEDGKESRISVQERQVTDSIALRPLV

SEQ ID NO: 262 - protein, 172 aa, Artificial Sequence
  translation of mouse CD40-4 variant 5
QCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICEKVVKKP

SEQ ID NO: 263 - protein, 123 aa, Artificial Sequence
  translation of mouse CD40-3 variant 5
QCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCH

SEQ ID NO: 264 - protein, 234 aa, Artificial Sequence
  partial human IgG1 Fc domain
DLEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - DNA, 1923 bp, Homo sapiens
cttctgtgtgtgcacatgtgtaatacatatctgggatcaaagctatctatataaagtccttgattctgtgtgggttcaaacacatttcaaagcttcaggatcctgaaaggttttgctctacttcctgaagacctgaacaccgctcccataaagccatggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgctaaagaaaagaagccctcttacaacaggggtctatgtgaaaatgcccccaacagagccagaatgtgaaaagcaatttcagccttattttattcccatcaattgagaaaccattatgaagaagagagtccatatttcaatttccaagagctgaggcaattctaacttttttgctatccagctatttttatttgtttgtgcatttggggggaattcatctctctttaatataaagttggatgcggaacccaaattacgtgtactacaatttaaagcaaaggagtagaaagacagagctgggatgtttctgtcacatcagctccactttcagtgaaagcatcacttgggattaatatggggatgcagcattatgatgtgggtcaaggaattaagttagggaatggcacagcccaaagaaggaaaaggcagggagcgagggagaagactatattgtacacaccttatatttacgtatgagacgtttatagccgaaatgatcttttcaagttaaattttatgccttttatttcttaaacaaatgtatgattacatcaaggcttcaaaaatactcacatggctatgttttagccagtgatgctaaaggttgtattgcatatatacatatatatatatatatatatatatatatatatatatatatatatatatatatatattttaatttgatagtattgtgcatagagccacgtatgtttttgtgtatttgttaatggtttgaatataaacactatatggcagtgtctttccaccttgggtcccagggaagttttgtggaggagctcaggacactaatacaccaggtagaacacaaggtcatttgctaactagcttggaaactggatgaggtcatagcagtgcttgattgcgtggaattgtgctgagttggtgttgacatgtgctttggggcttttacaccagttcctttcaatggtttgcaaggaagccacagctggtggtatctgagttgacttgacagaacactgtcttgaagacaatggcttactccaggagacccacaggtatgaccttctaggaagctccagttcgatgggcccaattcttacaaacatgtggttaatgccatggacagaagaaggcagcaggtggcagaatggggtgcatgaaggtttctgaaaattaacactgcttgtgtttttaactcaatattttccatgaaaatgcaacaacatgtataatatttttaattaaataaaaatctgtggtggtcgttttaaaaaaaaaaaaaaaaaaaaaaaaaaaaaaaaaaaaaaaaaaaaaaaaaaaaaaaaaa

SEQ ID NO: 266 - protein, 174 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

(19) United States
(12) Patent Application Publication
(10) Pub. No.: US 2014/0120091 A1
(43) Pub. Date: May 1, 2014

(54) FUSION PROTEINS FOR THERAPY OF AUTOIMMUNE AND CARDIOVASCULAR DISEASE

(71) Applicant: UNIVERSITY OF WASHINGTON THROUGH ITS CENTER FOR COMMERCIALIZATION, SEATTLE, WA (US)

(72) Inventors: Jeffrey A LEDBETTER, Shoreline, WA (US); Martha LEDBETTER, Shoreline, WA (US); Vince MONTES, Seattle, WA (US); Alan CHAIT, Seattle, WA (US); Sonya SLATER, Seattle, WA (US)

(21) Appl. No.: 14/069,216

(22) Filed: Oct. 31, 2013

Related U.S. Application Data
(60) Provisional application No. 61/720,925, filed on Oct. 31, 2012.

(51) Int. Cl. C07K 16/46; C07K 14/47; C07K 14/705

(52) U.S. Cl. CPC C07K 16/46; C07K 14/70578; C07K 14/4703
USPC 424/134.1; 530/387.3; 536/23.4; 435/320.1; 435/69.6; 530/350; 536/23.5; 435/69.1; 514/1.9; 514/1.1; 435/69.2; 435/328; 435/354; 435/366; 435/365; 435/367; 435/361; 435/370

(57) ABSTRACT

The present invention provides improved fusion proteins for therapy of autoimmune and cardiovascular disease.

### CROSS REFERENCE

This application is related to U.S. provisional patent application Ser. No. 61/720,925, filed Oct. 31, 2012, the disclosure of which is incorporated by reference.

### BACKGROUND

Cardiovascular disease is the leading cause of mortality in many nations, accounting for approximately 16.7 million deaths each year world-wide (1). The most common consequences of cardiovascular disease are myocardial infarction and stroke, which have a common underlying etiology of atherosclerosis. Atherosclerosis is promoted by low density lipoprotein (LDL) cholesterol, an atherogenic lipoprotein particle. The mainstay of LDL cholesterol lowering lies in the use of statin medications. The statins are 3-hydroxy-3-methyl-glutaryl-CoA (HMG CoA) reductase inhibitors, which block the rate-limiting step of endogenous cholesterol metabolism. They have been shown to reduce cardiovascular disease and improve survival (2). In addition, there are other drugs that lower LDL cholesterol, including niacin, ezetimibe and bile acid sequestrants.

Despite the various options for treating elevations in LDL cholesterol, there are problems associated with each of the therapies. The main reasons are intolerance due to adverse events. With the statins, many people cannot take them due to induction of myalgias or possibly due to existing liver disease. And in many cases of existing cardiovascular disease, the LDL is not lowered to goal levels with the use of statins alone or in conjunction with some of the other available medications. Thus, there is a need for therapies that can induce marked reductions of LDL cholesterol, with or without statins.

CD40-CD40 ligand (CD40L) binding has been implicated in diseases having an immune or autoimmune connection or heart disease. Animal models of immune-related disease in which the CD40-CD40L pathway has been demonstrated to play a role in the pathology include, for example, murine models of systemic lupus erythematosis (Lupus or SLE), arthritis (collagen-induced arthritis), multiple sclerosis (experimental autoimmune encephalomyelitis, EAE), autoimmune thyroiditis (experimental autoimmune thyroiditis, EAT), colitis (hapten-induced colitis), atherosclerosis and coronary artery disease, and allograft rejection. There is a need for improved CD40 ligand binding proteins with increased binding properties and capable of better expression and stability.

### SUMMARY OF THE INVENTION

In a first aspect, the present invention provides an isolated fusion protein comprising: a) a first binding domain N-terminal to an immunoglobulin constant region (Fc) domain; and b) a second binding domain C-terminal the Fc domain; wherein the first binding domain and the second binding domain are selected from the group consisting of: a proprotein convertase subtilisin/kexin type 9 (PCSK9) inhibitor and a CD40 extracellular domain (CD40 EC). The PCSK9 inhibitor of the fusion protein can be selected from the group consisting of: a low-density lipoprotein receptor epidermal growth factor-like repeat AB domain (LDLR EGF-AB) and an antigen binding fragment from a PCSK9 binding antibody. The Fc domain of the isolated fusion protein can be selected from the group consisting of: wildtype human IgG1 Fc domain, mutant human IgG1 Fc domain, wildtype human IgG4 Fc domain, mutant human IgG4 Fc domain. The isolated fusion protein can comprise the LDLR EGF-AB domain, wherein the LDLR EGF-AB domain comprises wildtype or mutant human LDLR EGF-AB domain. The isolated fusion protein of the invention can comprise a CD40 EC domain wherein the CD40 EC domain comprises wildtype or mutant human CD40 EC domain.

The inventors have discovered that the isolated fusion proteins of the invention can be used, for example, to reduce serum low density lipoprotein cholesterol levels and to treat cardiovascular disease, atherosclerosis, acute coronary syndrome, and autoimmune disorders, including but not limited to systemic lupus erythematosus, rheumatoid arthritis and other diseases where LDL levels are increased. The fusion proteins of the present disclosure are particularly effective in the treatment and/or management of cholesterol levels. In this regard, it will be appreciated that the fusion proteins of the present invention may be used to control, suppress, modulate, treat, or reduce unwanted levels of cholesterol. In yet other embodiments of the invention, the fusion protein may be used to treat disorders, diseases, etc. related to unhealthy levels of cholesterol.

In a second aspect, isolated fusion protein of the invention further comprising a third binding domain, wherein the third binding domain comprises a Cytotoxic T-Lymphocyte Antigen 4 (CTLA4).

In a third aspect, the present invention provides an isolated nucleic acid encoding the isolated fusion protein of the invention.

In a fourth aspect, the present invention provides a recombinant expression vector comprising the nucleic acid encoding the isolated fusion protein of the invention.

In a fifth aspect, the present invention provides a host cell comprising the recombinant expression vector encoding the isolated fusion protein of the invention.

In a sixth aspect, the present invention provides a method for producing the isolated fusion protein of the invention, comprising: (a) culturing the host cell comprising the recombinant expression vector encoding the isolated fusion protein of the invention under conditions suitable for expression of the nucleic-acid encoded fusion protein; and (b) isolating the fusion protein from the cultured cells.

In a seventh aspect, the present invention provides a pharmaceutical composition, comprising: (a) the isolated fusion protein of the invention; and (b) a pharmaceutically acceptable carrier.

In an eighth aspect, the present invention provides a method for treating coronary artery disease, comprising administering to a subject in need thereof the isolated fusion protein of the invention or the pharmaceutical composition of the invention, wherein the fusion protein or pharmaceutical composition is administered in an amount effective to treat coronary artery disease.

In a ninth aspect, the present invention provides an isolated mutant human CD40 EC domain protein comprising one or more amino acid substitutions selected from the group consisting of: K46H, K46T, E64Y, E64S, E66T, D69Q, E74T, H76Q, K81S, K81H, K81T, K81R, P85Y, P85W, N86T, N86Q, Q93S, T112Y, T112S, T112K, E114N, E114R, A115V, E117Q and L121P relative to the wildtype human CD40. The isolated mutant human CD40 EC domain can comprises one or more mutations selected from the group consisting of: (i) E64S; (ii) E64Y; (iii) E66T; (iv) K81S; (v) K81T; (vi) T112Y; (vii) E64S and K81S; (viii) K81H and L121P; (ix) E114N and E117Q; (x) E64Y, K81T and P85Y; (xi) E64S, K81H and L121P; and (xii) E64Y, K81T and P85Y.

The inventors have surprisingly discovered that the mutant human CD40 EC domain of the present invention has improved properties of increased expression levels in in vitro systems and increased binding properties to CD40L. These novel mutant human CD40 EC domain proteins could be used as a method for treating an autoimmune disease or coronary artery disease/heart disease in a subject.

In a tenth aspect, the present invention provides an isolated nucleic acid encoding the isolated mutant human CD40 EC domain of the invention.

In an eleventh aspect, the present invention provides a recombinant expression vector comprising the nucleic acid encoding the isolated mutant human CD40 EC domain of the invention.

In a twelfth aspect, the present invention provides a host cell comprising the recombinant expression vector encoding the isolated mutant human CD40 EC domain of the invention.

In a thirteenth aspect, the present invention provides method for producing the isolated mutant human CD40 EC domain of the invention, comprising: (a) culturing the host cell comprising the recombinant expression vector encoding the isolated mutant human CD40 EC domain of the invention under conditions suitable for expression of the nucleic-acid encoded protein; and (b) isolating the protein from the cultured cells.

In a fourteenth aspect, the present invention provides a pharmaceutical composition, comprising: (a) the isolated mutant human CD40 EC domain of the invention; and (b) a pharmaceutically acceptable carrier.

In a fifteenth aspect, the present invention provides a method for treating atherosclerosis in a subject, comprising administering to a subject in need thereof the isolated mutant human CD40 EC domain or pharmaceutical composition of the invention, wherein the protein or pharmaceutical composition of the invention is administered in an amount effective to inhibiting atherosclerotic plaque destabilization.

In a sixteenth aspect, the present invention provides a method for treating an autoimmune disease in a subject, comprising administering to a subject in need thereof isolated mutant human CD40 EC domain or pharmaceutical composition of the invention, wherein the protein or pharmaceutical composition is administered in an amount effective to inhibiting an autoimmune response.

### BRIEF DESCRIPTION OF THE DRAWINGS

The disclosed exemplary aspects have other advantages and features which will be more readily apparent from the detailed description, the appended claims, and the accompanying figures. A brief description of the figures is below.

FIG. 1A shows a schematic diagram of some of the preferred aspects for fusion genes and proteins described. FIG. 1B shows a schematic diagram of some of the preferred aspects for fusion genes and proteins described herein.

FIG. 2 shows a diagram of the subdomains of several of the preferred embodiments, indicating which domains are present in the constructs of interest.

FIG. 3A and FIG. 3B shows the predicted nucleotide and amino acid sequence of the hLDLR EGF-AB-hIgG1 with wild type or mutant sequence for the LDLR EGF-AB domain as indicated, and with mutations in the Fc domain of the Ig tail at P238S and P331S to reduce FcR binding and effector functions. The sequences shown in FIG. 3 correspond to SEQ ID NOs: 17, 18, 19, 20, 21, 22, 23 and 24.

FIG. 4 shows Western blot analysis of LDLR EGR-ABmthIgG 1 expressing culture supernatants immunoprecipitated with protein A agarose, and subjected to NuPAGE® (Life Technologies) gel electrophoresis, blotted to nitrocellulose, and probed with HRP-conjugated goat-anti-human IgG.

FIG. 5 shows a Western blot of CD40 EC-Ig-LDLR EGF-AB and LDLR EGF-AB-Ig-CD40 EC multispecific fusion proteins immunoprecipitated from transfected COS7 culture supernatants using protein A agarosse. The fusion proteins were eluted into reducing sample buffer and subjected to NuPAGE® gel electrophoresis, blotted to nitrocellulose and probed with HRP-conjugated goat anti-human IgG.

FIG. 6 shows a Western blot of CD40Ig fusion proteins immunoprecipitated from transfected COS7 culture supernatants using protein A agarose. The fusion proteins were eluted into reducing sample buffer and subjected to NuPAGE® gel electrophoresis, blotted to nitrocellulose and probed with HRP-conjugated goat anti-human IgG.

FIG. 7A shows the SDS-PAGE gel analysis of the LDLR EGF-AB-mthIgG1 (WT and MT) under nonreducing conditions. FIG. 7B shows the same proteins subjected to SDS-PAGE electrophoresis under reducing conditions. FIG. 7 also shows the migration pattern of several preferred embodiments for the hCD40 EC-mthIgG fusion proteins under both reducing and nonreducing conditions.

FIG. 8 shows results from a PCSK9 antigen capture ELISA using plates coated with the LDLR EGF-ABmthIgG1 fusion proteins (WT, MT) or CD40IgG fusion proteins at 1 ug/ml in 0.1 M Na-carbonate buffer, pH 9.6. Serial dilutions of recombinant human PCSK9-his6 (AcroBiosystems) were added, and binding detected by reaction with a horseradish peroxidase conjugated anti-his6 antibody.

FIG. 9 shows results from a PCSK9 antigen capture ELISA using plates coated with the LDLR EGF-ABmthIgG1 fusion proteins, in the presence of 5 mM CaCl2 and MgCl2, or EDTA in PBS, and at a neutral pH, acidic pH (NaOAc buffer, pH 5.2), or basic pH (Nacarbonate buffer, pH 9.6). The binding activity of the mutant form of the EGF-AB domain showed a more marked increase in the presence of divalent cations (calcium) and at a more acidic pH.

FIG. 10 shows that PCSK9 antigen immobilized on ELISA plates is able to capture the LDLR EGF-ABmthIgG fusion proteins from solution.

FIG. 11 shows that human LDLR EGF-ABmthIgG fusion proteins immobilized on ELISA plates are able to bind to and capture murine PCSK9-his fusion protein from solution, indicating that the binding epitopes are conserved between species.

FIG. 12 shows results of an assay exploring the effect of the LDLR EGF-AB Ig fusion proteins on blood cholesterol levels using an in vivo mouse model.

FIG. 13 shows alignment of human CD40 truncation variants, and the truncations of the extracellular domain constructed and tested as -Ig fusion proteins (truncation human CD40-3 protein is SEQ ID NO: 257; truncation human CD40-4S protein is SEQ ID NO 258; truncation human CD40-4L protein is SEQ ID NO 259; truncation human CD40 variant 1 is SEQ ID NO 260).

FIG. 14 shows the alignment of several mouse CD40 transcript variants, and the truncations of the extracellular domain for the mouse CD40Ig fusion proteins described herein (truncation of mouse CD40 variant 5 is SEQ ID NO: 261; truncation of mouse CD40-4 variant 5 is SEQ ID NO: 262; and truncation of mouse CD40-3 variant 5 is SEQ ID NO: 263).

FIG. 15 shows the predicted nucleotide and amino acid sequence of a preferred embodiment of the CD40 extracellular domain, identified as hCD40-4s EC-mthIgG fusion gene and protein. The sequences shown in FIG. 15 correspond to SEQ ID NOs: 49 and 50.

FIG. 16 shows a schematic diagram of how human Jurkat cells were panned on plates coated with immobilized CD40Ig in order to select for cells expressing higher levels of CD40L. FIG. 16 shows the binding of different purified hCD40Ig fusion proteins to a derivative of human Jurkat cells expressing higher levels of CD40L (CD154).

FIG. 17 shows the relative binding activity of CD40Ig, CD40IgCD40, and CTLA4IgCD40 fusion proteins to CD40L on Jurkat cells. Serial dilutions of each purified fusion protein were incubated with Jurkat cells, washed, and binding detected with AF647 conjugated goat anti-human IgG antibody.

FIG. 18 shows blocking activity of the different hCD40Ig fusion proteins bound to Jurkat cells, and then incubated with a fluorescent conjugate of CD40Ig. The CD40IgCD40 fusion protein exhibited greater blocking activity than did the other CD40Ig fusion proteins.

FIG. 19 shows the results of a CD40L binding ELISA assay where human CD40L (Peprotech) was immobilized at 2 ug/ml, purified CD40 EC domain containing fusion proteins were serially diluted, and binding detected with a horseradish peroxidase conjugated goat anti-human IgG antibody.

FIG. 20 shows results of an exploratory platelet aggregation assay using different concentrations of collagen and numbers of platelets to determine the experimental conditions required for platelet aggregation. Platelet aggregation was monitored over the course of a 20 minute time span with constant agitation using a SYNERGY®™ 2 platereader (Biotek, VT) and the aggregation traces plotted as a function of time.

FIG. 21 shows the results of a platelet aggregation assay using collagen mediated aggregation (5 ug/ml) as the positive control, and comparing the effects of CD40 or CD40L targeted fusion proteins and antibodies on platelet activation and aggregation. Different CD40Ig fusion proteins, a monoclonal CD154 specific antibody, or immune complexes of recombinant human CD154 and the CD154 specific antibody were compared for their ability to mediate platelet aggregation under identical conditions. As shown in the figure, although the CD154 antibody mediates some platelet aggregation, the aggregation is greatly increased when soluble CD154 is incubated with the antibody to form an immune complex prior to the assay.

FIG. 22 shows results of platelet aggregation assay comparing the effects of CD40 EC WThIgG and CD40 EC MThIgG fusion proteins. Neither fusion protein mediated platelet aggregation although collagen at 5 ug/ml did so.

FIG. 23 shows results of a platelet aggregation assay where the platelets were preincubated with CD40Ig fusion proteins for 30 minutes prior to addition of CD154.

FIG. 24 shows a fusion protein antigen binding ELISA where soluble mouse anti-human CD40 (5 ug/ml) was immobilized onto ELISA plates, incubated with culture supernatants containing CD40-Ig-LDLR-EGF-AB fusion proteins, and binding detected with soluble PCSK9-his6, followed by an HRP-anti-his6 tag antibody, demonstrating that the fusion proteins are able to bind at both ends simultaneously.

FIG. 25 shows results from two different ELISAs for levels of expression and binding to CD40L (CD154) of the various mutant CD40 EC-Ig fusion proteins indicated. The IgG sandwich ELISA estimated the amount of fusion protein present in the supernatants, while the CD40L antigen binding ELISA estimated the relative binding activity of each fusion protein cultures. The assays were performed three times on COS supernatants from repeated transfections with similar results.

FIG. 26 shows the ratio between the CD40L binding activity of each mutant CD40 EC-Ig fusion protein indicated and the level of IgG expression levels. The level of CD40 EC-Ig fusion protein detected in the IgG sandwich ELISA was used to normalize the amount of CD40L binding detected for each clone.

FIG. 27 shows the relative binding of each mutant CD40 EC-Ig fusion protein to CD40L (CD154) on Jurkat cells compared to wildtype CD40 EC-Ig fusion protein.

FIG. 28 shows results from two different ELISAs for levels of expression and binding to CD40L (CD154) of the various mutant CD40 EC-Ig fusion proteins indicated. The IgG sandwich ELISA estimated the amount of fusion protein present in the supernatants, while the CD40L antigen binding ELISA estimated the relative binding activity of each fusion protein cultures. The assays were performed three times on COS supernatants from repeated transfections with similar results.

FIG. 29 shows the ratio between the CD40L binding activity of each mutant CD40 EC-Ig fusion protein indicated and the level of IgG expression levels. The level of CD40 EC-Ig fusion protein detected in the IgG sandwich ELISA was used to normalize the amount of CD40L binding detected for each clone.

FIG. 30 shows a schematic numbering of a portion of human IgG1 Fc containing a hinge region (˜216-229) with potential substations of Cysteine to Serine at positions 220, 226 and 229; a CH2 domain (˜230-340) with potential substitutions of Proline to Serine at positions 238 and 331; and a CH3 domain (˜341-447). The sequence shown in FIG. 30 is SEQ ID NO: 264.

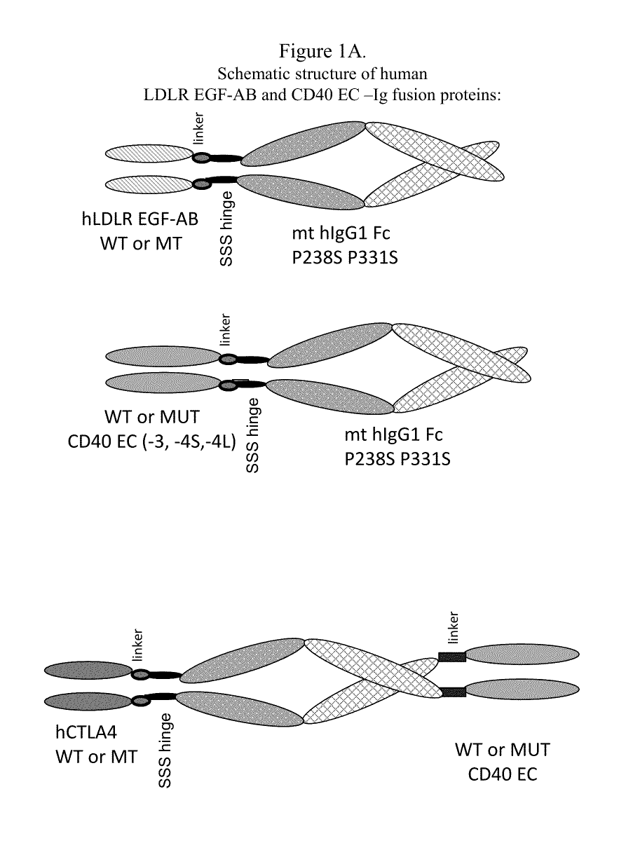

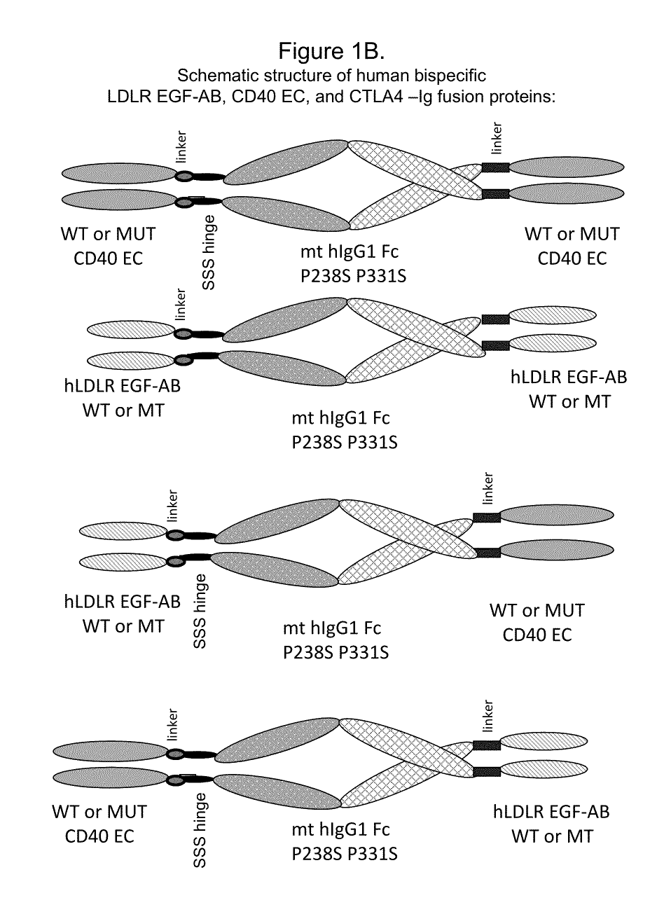

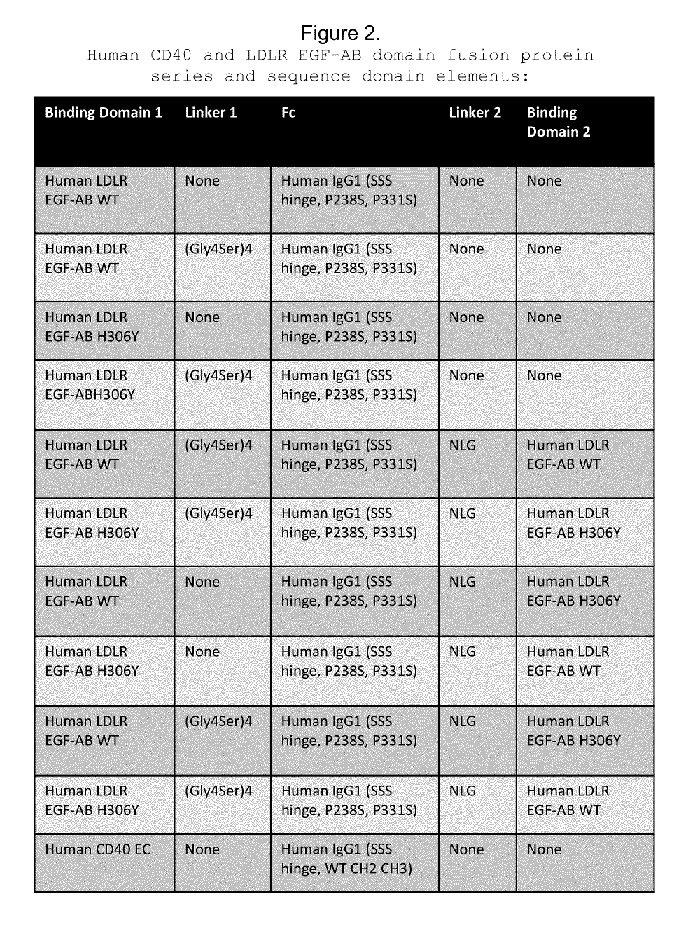

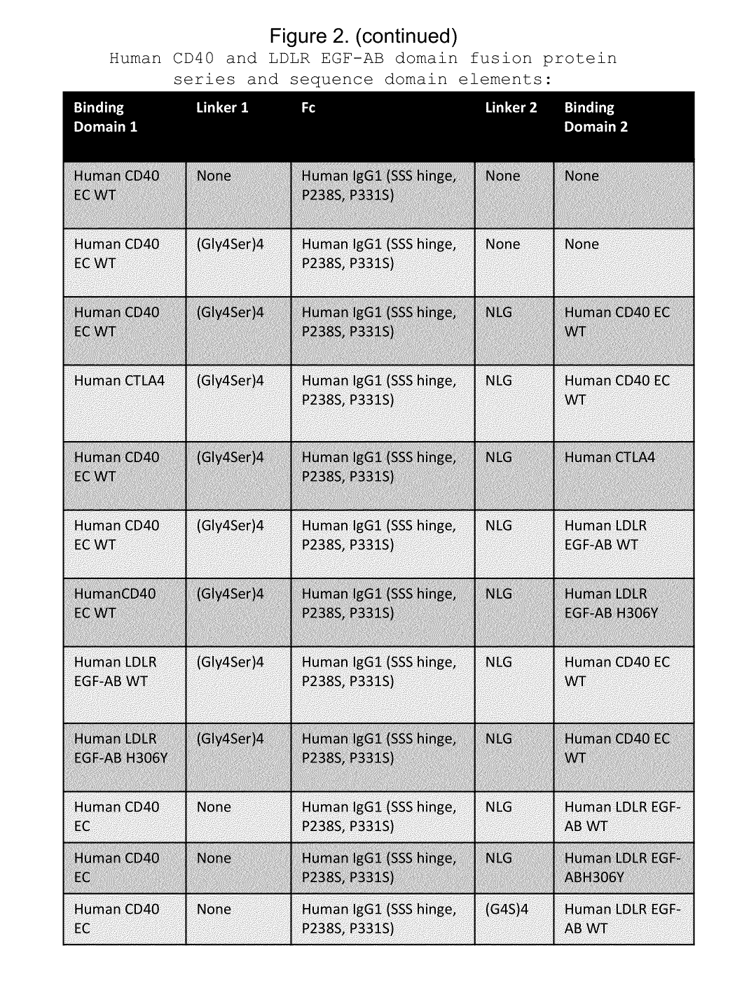

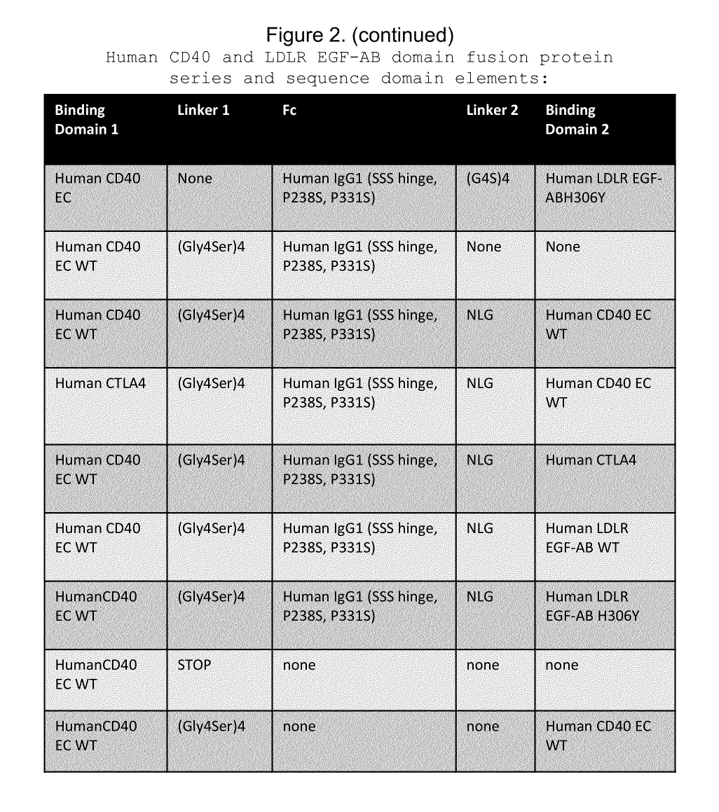

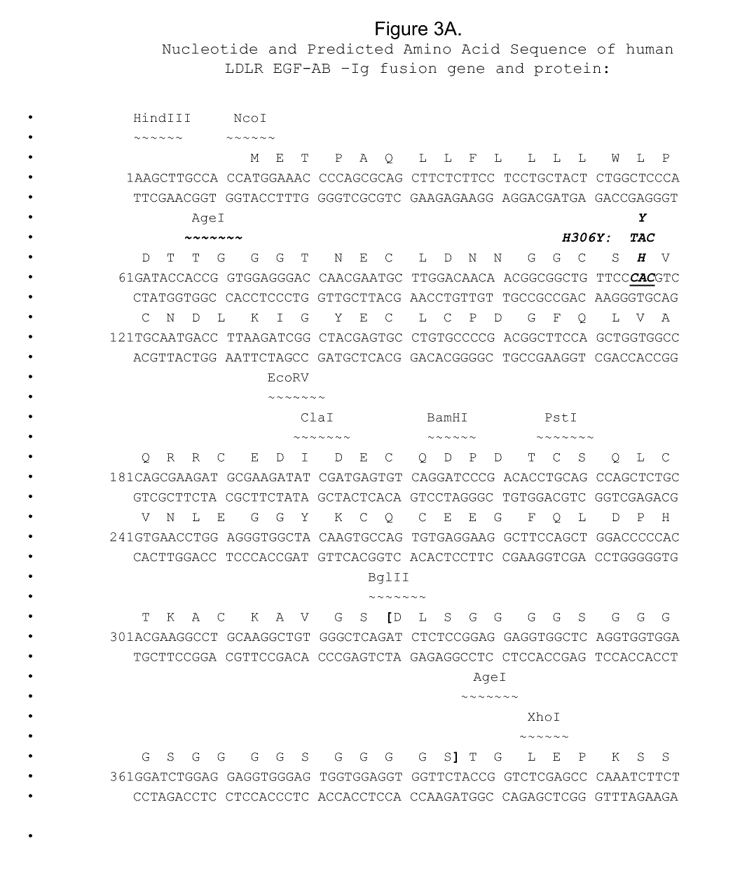

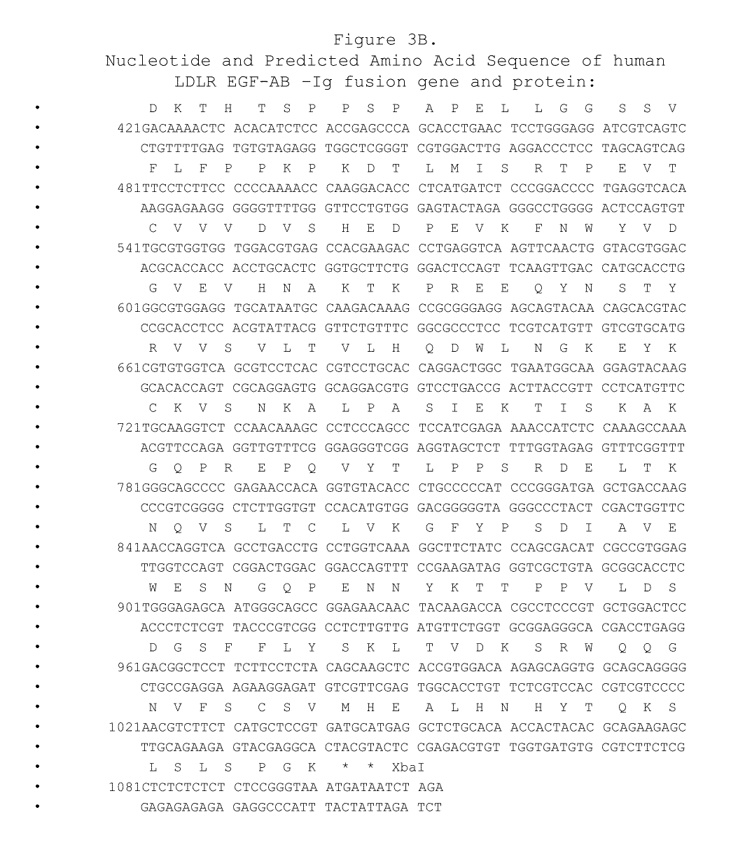

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

Terms used in the claims and specification are defined as set forth below unless otherwise specified. In the case of direct conflict with a term used in a parent provisional patent application, the term used in the instant specification shall control.

As used herein, the singular forms “a”, “an” and “the” include plural referents unless the context clearly dictates otherwise. “And” as used herein is interchangeably used with “or” unless expressly stated otherwise.

All embodiments disclosed herein can be used in combination unless the context clearly dictates otherwise.

In a first aspect, the present invention provides an isolated fusion protein comprising: a) a first binding domain N-terminal to an immunoglobulin constant region (Fc) domain; and b) a second binding domain C-terminal the Fc domain; wherein the first binding domain and the second binding domain are selected from the group consisting of: a proprotein convertase subtilisin/kexin type 9 (PCSK9) inhibitor and a CD40 extracellular domain (CD40 EC).

The inventors have discovered that the isolated fusion proteins of the invention can be used, for example, to reduce serum low density lipoprotein cholesterol levels and to treat cardiovascular disease, atherosclerosis, acute coronary syndrome, and autoimmune disorders, including but not limited to systemic lupus erythematosus, rheumatoid arthritis and other diseases where LDL levels are increased. The fusion proteins of the present disclosure are particularly effective in the treatment and/or management of cholesterol levels. In this regard, it will be appreciated that the fusion proteins of the present invention may be used to control, suppress, modulate, treat, or reduce unwanted levels of cholesterol. In yet other embodiments of the invention, the fusion protein may be used to treat disorders, diseases, etc. related to unhealthy levels of cholesterol.

As used herein, the term “PCSK9 inhibitor” refers to any suitable inhibitor that is capable of preventing the proprotein convertase subtilisin/kexin type 9 protein (PCSK9) protein from binding to low-density lipoprotein receptor (LDLR). In particular, the PCSK9 inhibitor prevents PCSK9 from binding to endogenous epidermal growth factor-like repeat A (EGF-A) domain of the low-density lipoprotein receptor (LDLR). Fusion proteins that block PCSK9 can lower cholesterol. The PCSK9 inhibitor can be selected from the group consisting of: a low-density lipoprotein receptor epidermal growth factor-like repeat AB domain (LDLR EGF-AB) and mutated version thereof that retain PCSK9 binding activity, an antigen binding fragment from a PCSK9 binding antibody or mutated version thereof that retains PCSK9 binding activity, and a peptide PCSK9 binder.

An “antigen binding fragment” as disclosed herein refers to a protein fragment that contains at least the complementary determining regions (CDRs) of an antibody heavy or light chain from which the antigen binding fragment was derived. An antigen binding fragment is capable of binding to a target antigen i.e., PCSK9. For example the CDRs or the entire variable regions of the heavy and light chains of an antibody can be fused together to form a single-chain variable fragment (scFv).

As used herein, the term “LDLR EGF-AB domain” refers to the human low-density lipoprotein receptor epidermal growth factor-like repeat AB domain (NCBI reference accession number for the human LDLR, transcript variant 2, is NCBI NM_{—}001195798.1 (SEQ ID NO: 01), and the GeneBank accession number for the human LDLR mRNA is AY114155.1. The associated full-length protein sequence is AAM56036.1 (SEQ ID NO: 02). The amino acid sequence LDLR EGF-AB domain corresponds to SEQ ID NO: 04 and has high binding activity for PCSK9, with both the wild type and the mutant forms exhibiting similar levels of binding activity for PCSK9 and PCSK9-his6 fusion proteins. The level of binding can be altered by adjustments in pH or divalent cation levels. In the presence of calcium and acidic pH, the mutant form exhibits higher binding activity for PCSK9 than does the wild type form of the fusion protein. The LDLR EGF-AB domain can comprise a mutant human LDLR EGF-AB domain having one or more amino acid substitutions that increase the stability of the LDLR EGF-AB domain and/or increase binding affinity of LDLR EGF AB domain to a PCSK9 antigen. For example, the LDLR EGF-AB domain can comprise a mutant human LDLR EGF-AB domain having an amino acid change of H306Y relative to the wildtype human low-density lipoprotein receptor. Additional mutant human LDLR EGF-AB domains include, but are not limited to, D299S, D299A, D299K, N301L, N309R, N309K, D310K or any of the variants described in Zhang et al., (2012) Journal of Molecular Biology 422:685-96.

As used herein the term “immunoglobulin constant region (Fc) domain” refers to the constant region of an immunoglobulin. For example the Fc domain described here can be the wildtype or a mutated constant domain from IgG1, IgG2, IgG3 or IgG4 of human. For example, in one embodiment, the human IgG1 Fc domain of the invention comprises a hinge region, a CH2 domain and a CH3 domain. In another embodiment, the human IgG1 Fc domain can contain mutations of residues C220, C226, C229 in the hinge region (C to S) and mutations of residues P238 and P331 (P to S) in the CH2 domain. The Fc domain can be human IgG1 with the three cysteines of the hinge region (C220, C226, C229 relative to wildtype human IgG1) each changed to serine, and the proline at position 238 of the CH2 domain changed to serine, and the proline at position 331 of the CH2 domain changed to serine. In another embodiment, the Fc domain can be human IgG1 with N297 changed to any other amino acid, since mutating N297 eliminates the N-linked glycosylation site. In another embodiment, the Fc domain can be human IgG1 with one or more amino acid change between positions 292 and 300 relative to wildtype human IgG1. In another embodiment, the Fc domain can be human IgG1 with an amino acid addition or deletion at any position between residues 292 and 300 relative to wildtype human IgG1. In another embodiment, the Fc domain is human IgG1 with an SCC hinge (C220S, C226, C229); in a further embodiment an SSS (C220S, C226S, C229S) hinge. In further embodiments, the Fc domain can be human IgG1 with an SCC hinge and P238S/P331S mutations; with an SCC hinge and P238S/K322S/P331S mutations; an SSS hinge and P238S/P3315 mutations; or an SSS hinge and P238S/K322S/P3315 mutations. In another embodiment, the Fc domain can be human IgG1 with mutations that alter binding by Fc gamma receptors (I, II, III) without affecting FcRn binding important for half-life. These mutations are designed to prevent binding of C1q and prevent binding to Fc gamma receptors. The isolated protein fused to human IgG1 Fc retains the ability to bind to Protein A (useful for purification) and binding to FcRN (important for long serum half-life). The Fc portion of the human IgG1 heavy chain polypeptide has the ability to self-associate, a function which facilitates the formation of dimers.

In an embodiment the fusion protein is oriented from N-terminal to C-terminal as first binding domain-Fc domain-second binding domain. In another embodiment the fusion protein is oriented from N-terminal to C-terminal as first binding domain-second binding domain-Fc domain. In yet another embodiment the fusion protein is oriented from N-terminal to C-terminal as first binding domain second binding domain-first binding domain-Fc domain. In a yet another embodiment the fusion protein is oriented from N-terminal to C-terminal in any combination of first binding domain, second binding domain and Fc domain.

The term “multimer” as used herein refers to the interaction of two or more of the fusion proteins through the Fc domain to form a larger complex. A multimer can be a dimer or homodimer a single fusion protein embodiment of the invention or a heterodimer of two different fusion protein embodiments of the invention. Furthermore, a multimer can be an interaction between 3, 4, 5, 6, or more fusion proteins of the invention to form a larger complex.

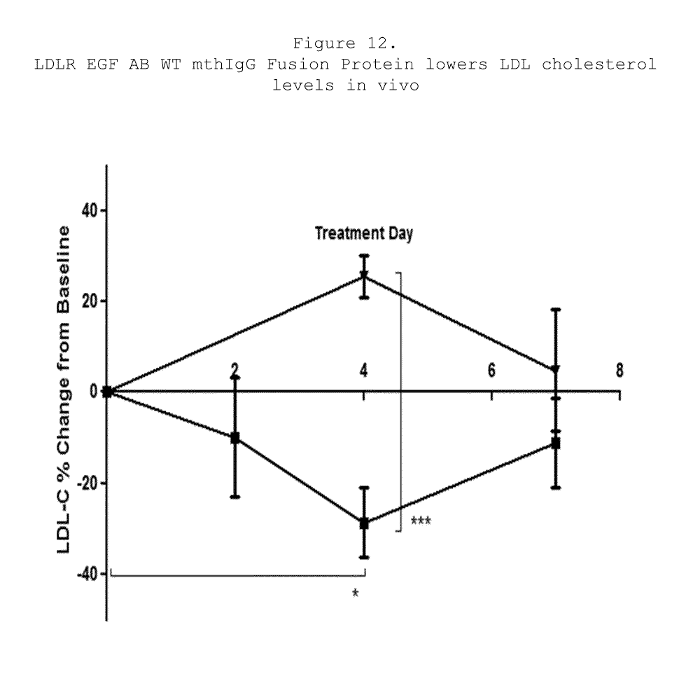

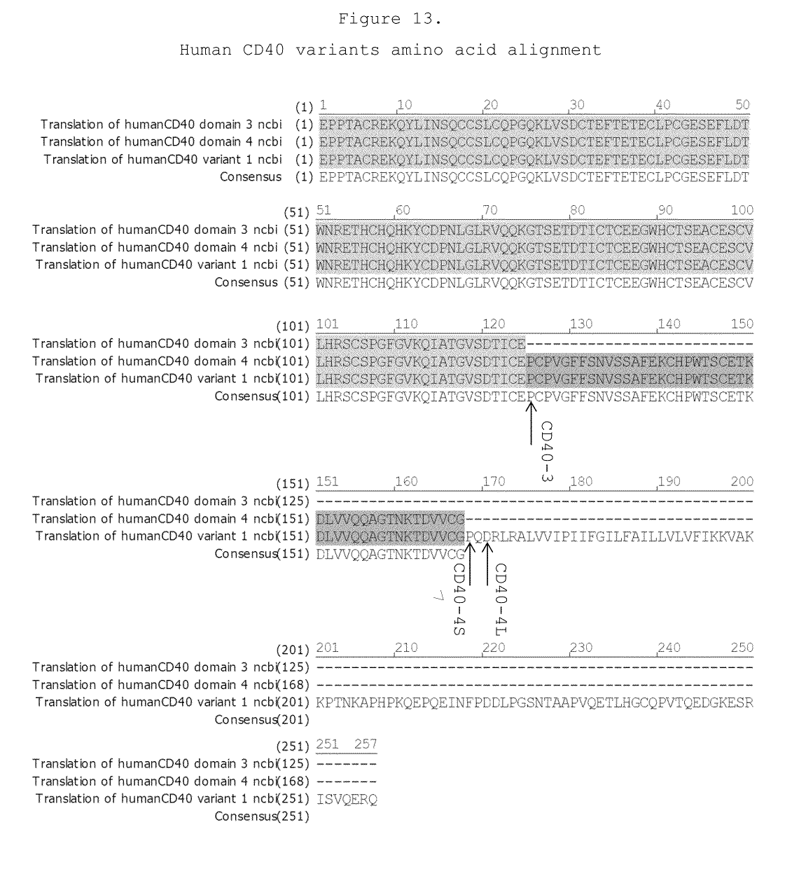

The term “CD40 EC domain” as used herein, refers to a domain within the human full length CD40 (SEQ ID NO: 40) from about amino acid 21 to about amino acid 277 of the human full length CD40 (for example SEQ ID NO: 260 comprises the CD40 EC domain), or C terminal truncations including, amino acid 21 to about amino acid 190 (or position 170, from about amino acid 21 to about amino acid 188 (or position 168 of the truncated, processed form shown in FIG. 13) or from about amino acid 21 to about amino acid 145 (or position 125 of the truncated, processed form shown in FIG. 13). Examples of C-terminal truncated human CD40 EC domain are shown in FIG. 13. Human CD40, also called tumor necrosis factor receptor superfamily, member 5, (NCBI reference accession number: NM_{—}001250.4, human CD40 transcript variant 1; SEQ ID NO: 39). The associated full-length protein sequence is NP_{—}001241 (SEQ ID NO: 40). The human CD40 EC domain retains binding activity for human CD40 ligand (CD40L, or CD154) when truncated at several locations (e.g., residues 21-145 or 21-188 or 21-190, as shown in FIG. 13 where the numbering begins with the first amino acid of full length CD40, relative to wildtype full length human CD40). The CD40 EC domain of the invention can comprise a mutant human CD40 EC domain having one or more amino acid substitutions that increase the stability of the CD40 EC domain and/or increase binding affinity of CD40 EC domain to CD40 ligand. For example, the amino acid substitutions that increase the stability of the CD40 EC domain and/or increase binding affinity of CD40 EC domain to CD40 ligand could be at one or more amino acid residues selected from K46, E64, E66, D69, E74, H76, Q79, K81, D84, P85, N86, Q93, H110, T112, E114, A115, E117 or L121 relative to the wildtype human CD40 (these mutants can also be made in the C-terminal truncated CD40 EC variant shown in FIG. 13). Furthermore, the mutant human CD40 EC domain with increased stability and/or increased binding affinity can comprise one or more amino acid substitutions including but not limited to: K46H, K46T, E64Y, E64S, E66T, D69Q, E74T, H76Q, K81S, K81H, K81T, K81R, P85Y, P85W, N86T, N86Q, Q93S, T112Y, T112S, T112K, E114N, E114R, A115V, E117Q and L121P. In particular, the mutant human CD 40 EC domain can comprise one or more mutations: (i) E64S; (ii) E64Y; (iii) E66T; (iv) K81S; (v) K81T; (vi) T112Y; (vii) E64S and K81S; (viii) K81H and L121P; (ix) E114N and E117Q; (x) E64Y, K81T and P85Y; (xi) E64S, K81H and L121P; and (xii) E64Y, K81T and P85Y.

In another embodiment of the invention, the isolated fusion protein can further comprise a third binding domain comprising a Cytotoxic T-Lymphocyte Antigen 4 (CTLA4). CTLA4 (NCBI RefSeq for mRNA NM_{—}001037631.2 [SEQ ID NO: 265]; NCBI RefSeq for protein NP_{—}001032720.1 [SEQ ID NO: 266]) is a member of the immunoglobulin superfamily and encodes a protein which transmits an inhibitory signal to T cells. The protein contains a V domain, a transmembrane domain, and a cytoplasmic tail. Alternate transcriptional splice variants, encoding different isoforms, have been characterized. The membrane-bound isoform functions as a homodimer interconnected by a disulfide bond, while the soluble isoform functions as a monomer. Mutations in this gene have been associated with insulin-dependent diabetes mellitus, Graves disease, Hashimoto thyroiditis, celiac disease, systemic lupus erythematosus, thyroid-associated orbitopathy, and other autoimmune diseases. CTL4A containing fusion proteins of the invention can be useful for inhibiting the costimulation of T cells and in the treatment of immune, autoimmune diseases and transplantation.

In a second aspect, the invention comprises an isolated nucleic acid encoding the isolated fusion protein of the invention. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In a third aspect, the invention comprises a recombinant expression vector comprising the nucleic acid encoding the isolated fusion protein of the invention. “Recombinant expression vector” includes vectors that operatively link a nucleic acid coding region or gene to any control sequences capable of effecting expression of the gene product. “Control sequences” operably linked to the nucleic acid sequences of the invention are nucleic acid sequences capable of effecting the expression of the nucleic acid fusion proteins. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The construction of expression vectors for use in transfecting prokaryotic cells is also well known in the art, and thus can be accomplished via standard techniques. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the host organisms either as an episome or by integration into host chromosomal DNA. In a preferred embodiment, the expression vector comprises a plasmid. However, the invention is intended to include other expression vectors that serve equivalent functions, such as viral vectors.

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene.

Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid fusion proteins comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. Construction of vectors according to the invention employs conventional ligation techniques. Isolated vectors or DNA fragments are cleaved, tailored, and re-ligated in the form desired to generate the required vector. If desired, sequence analysis to confirm that the correct sequences are present in the constructed vector is performed using standard methods. Suitable methods for constructing expression vectors, preparing in vitro transcripts, introducing DNA into host cells, and performing analyses for assessing expression and function are known to those skilled in the art. The presence of a gene sequence in a sample is detected, or its amplification and/or expression quantified by conventional methods, such as Southern or Northern analysis, Western blotting, dot blotting of DNA, RNA or protein, in situ hybridization, immunocytochemistry or sequence analysis of nucleic acid or protein fusion proteins. Those skilled in the art will readily envisage how these methods may be modified.

In a fourth aspect, the invention comprises a host cell comprising the recombinant expression vector encoding the isolated fusion protein of the invention. The present invention provides host cells that have been transfected with the recombinant expression vectors disclosed herein, wherein the host cells can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, N.Y.). Any eukaryotic cell can be useful as a host cell in the practice of producing the proteins of the invention. Examples include, but are not limited to, human fibroblast cells, and cells from other animals such as ovine, porcine, murine, bovine. Specific examples of mammalian cells include, but are not limited to COS, HeLa, CHO, DUX, B11, Sp2/0, W138, DHK, and HEPG2 cells

In a fifth aspect, the invention comprises a method for producing the isolated fusion protein of the invention, comprising: (a) culturing the host cell of a host cell comprising the recombinant expression vector encoding the isolated fusion protein of the invention under conditions suitable for expression of the nucleic-acid encoding the fusion protein; and (b) isolating the fusion protein from the cultured cells.

In an embodiment, this method comprises (i) transfecting the mammalian cell with a recombinant expression vector encoding the protein of the invention; (ii) culturing the mammalian cell transfected in step (i); and (iii) recovering the biologically active fusion protein produced by the cultured mammalian cell. The expressed fusion protein can be recovered from the cell free extract, but preferably they are recovered from the culture medium. Methods to recover fusion protein from cell free extracts or culture medium are well known to the person skilled in the art.

In an embodiment, a method for recovering the biologically active fusion protein comprises: (a) identifying the biologically active protein fusion protein by the presence of the molecular tag; and (b) separating the biologically active fusion protein having the molecular tag identified from fusion proteins without the molecular tag, so as to recover the biologically active fusion protein produced by the cultured mammalian cell.

Any eukaryotic cell can be useful in the practice of producing the proteins of the invention. Examples include human cells, for example fibroblast cells, and cells from other animals such as ovine, porcine, murine, bovine. Specific examples of mammalian cells include COS, HeLa, CHO, DUX, B11, Sp2/0, W138, DHK, and HEPG2 cells. Cells can be transfected with an expression vectors using methods known in the art, including but not limited to, chemical-based transfection (i.e. calcium phosphate, liposomes, cationic liposomes, DEAE-dextran, polyethylamine or dendrimers e.g., POLYFECT®), non-chemical methods (i.e., electroporation, optical transfection or gene gun) or viral based methods. Transfection of cells can be stable or transient transfection.

The fusion proteins can be linked to detectable markers for use in diagnosis, both in vivo and in vitro, and for use in therapy. Examples of detectable markers to which such fusion proteins can be linked include, but are not limited to, enzymes, paramagnetic ions or compounds, members of the avidin-biotin specific binding pair, fluorophores, chromophores, chemiluminophores, heavy metals, and radioisotopes.

In a sixth aspect, the invention provides a pharmaceutical composition comprising the isolated fusion protein of the invention and a pharmaceutically acceptable carrier.

In certain embodiments, an isolated fusion protein, as described herein, can be administered alone. In certain embodiments, the isolated protein can be administered prior to the administration of at least one other therapeutic agent. In certain embodiments, the isolated protein can be administered concurrent with the administration of at least one other therapeutic agent. In certain embodiments, the isolated protein can be administered subsequent to the administration of at least one other therapeutic agent. In other embodiments, the isolated protein can be administered prior to the administration of at least one other therapeutic agent. As will be appreciated by one of skill in the art, in some embodiments, the isolated protein can be combined with the other agent/compound. In some embodiments, the isolated protein and other agent can be administered concurrently. In some embodiments, the isolated protein and other agent are not administered simultaneously, with the isolated protein being administered before or after the agent is administered. In some embodiments, the subject receives both the isolated protein and the other agent during a same period of prevention, occurrence of a disorder, and/or period of treatment.

Pharmaceutical compositions of the invention can be administered in combination therapy, i.e., combined with other agents. In certain embodiments, the combination therapy comprises the isolated fusion protein, in combination with at least one other agent. Agents include, but are not limited to, in vitro synthetically prepared chemical compositions, antibodies, antigen binding regions, and combinations and conjugates thereof. In certain embodiments, an agent can act as an agonist, antagonist, allosteric modulator, or toxin.

In certain embodiments, the invention provides for pharmaceutical compositions comprising the isolated fusion protein together with a pharmaceutically acceptable diluent, carrier, solubilizer, emulsifier, preservative and/or adjuvant.

In certain embodiments, the invention provides for pharmaceutical compositions comprising the isolated fusion protein and a therapeutically effective amount of at least one additional therapeutic agent, together with a pharmaceutically acceptable diluent, carrier, solubilizer, emulsifier, preservative and/or adjuvant.

In certain embodiments, acceptable formulation materials preferably are nontoxic to recipients at the dosages and concentrations employed. In some embodiments, the formulation material(s) are for sub-cutaneous (s.c.) and/or intravenous (I.V.) administration. In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In some embodiments, the formulation comprises PBS; 20 mM NaOAC, pH 5.2, 50 mM NaCl; and/or 10 mM NAOAC, pH 5.2, 9% Sucrose.

In certain embodiments, the isolated fusion proteins, as described herein, and/or a therapeutic fusion protein can be linked to a half-life extending vehicle known in the art. Such vehicles include, but are not limited to, polyethylene glycol (PEG), glycogen (e.g., glycosylation of the fusion protein), and dextran. Such vehicles are described, e.g., in U.S. application Ser. No. 09/428,082, now U.S. Pat. No. 6,660,843 and published PCT Application No. WO 99/25044, which are hereby incorporated by reference for any purpose.

In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and rate of in vivo clearance of the antibodies of the invention.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore.

In certain embodiments, a composition comprising the fusion protein, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising the fusion protein, with or without at least one additional therapeutic agent, can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising a desired fusion protein, with or without additional therapeutic agents, in a pharmaceutically acceptable vehicle. In certain embodiments, a vehicle for parenteral injection is sterile distilled water in which a fusion protein presented herein, with or without at least one additional therapeutic agent, is formulated as a sterile, isotonic solution, properly preserved. In certain embodiments, the preparation can involve the formulation of the desired fusion protein with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired fusion protein.

In certain embodiments, it is contemplated that formulations can be administered orally. In certain embodiments, a fusion protein, as presented herein, with or without at least one additional therapeutic agents, that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of the fusion protein and/or any additional therapeutic agents. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of a fusion protein described herein, with or without at least one additional therapeutic agents, in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving a fusion protein described herein, with or without at least one additional therapeutic agent(s), in sustained- or controlled-delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly(2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al., Proc. Natl. Acad. Sci. USA, 82:3688-3692 (1985); EP 036,676; EP 088,046 and EP 143,949.

In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising a fusion protein described herein, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the fusion protein delivered, the indication for which a fusion protein described herein, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 μg/kg up to about 100 mg/kg; or 1 μg/kg up to about 100 mg/kg; or 5 μg/kg up to about 100 mg/kg.

In certain embodiments, the frequency of dosing will take into account the pharmacokinetic parameters of a fusion protein described herein and/or any additional therapeutic agents in the formulation used. In certain embodiments, a clinician will administer the composition until a dosage is reached that achieves the desired effect. In certain embodiments, the composition can therefore be administered as a single dose, or as two or more doses (which may or may not contain the same amount of the desired fusion protein) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device.

In certain embodiments, the composition can be administered locally via implantation of a membrane, sponge or another appropriate material onto which the desired fusion protein has been absorbed or encapsulated. In certain embodiments, where an implantation device is used, the device can be implanted into any suitable tissue or organ, and delivery of the desired fusion protein can be via diffusion, timed-release bolus, or continuous administration.

In certain embodiments, it can be desirable to use a pharmaceutical composition comprising a fusion protein, with or without at least one additional therapeutic agent, in an ex vivo manner. In such instances, cells, tissues and/or organs that have been removed from the patient are exposed to a pharmaceutical composition comprising a fusion protein, with or without at least one additional therapeutic agent, after which the cells, tissues and/or organs are subsequently implanted back into the patient.

In certain embodiments, a fusion protein and/or any additional therapeutic agents can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

In a seventh aspect, the invention comprises method for treating coronary artery disease and/or an immune disorder or autoimmune disease, comprising administering to a subject in need thereof one or more isolated fusion proteins of the invention or any of the pharmaceutical compositions of the invention, wherein the fusion protein or pharmaceutical composition is administered in an amount effective to treating coronary artery disease and/or an immune disorder or autoimmune disease.

As used herein, “coronary artery disease” refers to atherosclerotic heart disease, coronary heart disease, or ischemic heart disease (IHD) and is the most common type of heart disease and cause of heart attacks. The disease is caused by plaque building up along the inner walls of the arteries of the heart, which narrows the arteries and reduces blood flow to the heart. Coronary artery disease has a number of confirmed risk factors, including but not limited to, hypercholesterolemia (specifically, serum LDL concentrations), smoking, hypertension, hyperglycemia, and high levels of lipoprotein A, a compound formed when LDL cholesterol combines with a substance known as Apoliprotein A.

As used herein, “treating coronary artery disease” means accomplishing one or more of the following: (a) lowering low density lipoprotein (LDL) levels in the blood; (b) lowering cholesterol levels in the blood of a subject with the coronary artery disease; (c) lowering triglyceride levels in the blood of a subject with the coronary artery disease; (d) limiting or preventing development of symptoms characteristic of the coronary artery disease being treated; (e) inhibiting worsening of symptoms characteristic of coronary artery disease; (f) limiting or preventing recurrence of the coronary artery disease being treated in patients that have previously had the coronary artery disease; (g) limiting or preventing recurrence of symptoms in patients that were previously symptomatic for the coronary artery disease; (h) limiting development of the coronary artery disease in a subject at risk of developing the coronary artery disease; and (i) lowering the risk of chest pain, stroke, heart attack, or certain heart and blood vessel problems in people who have certain risk factors for heart disease or not yet showing the clinical effects of the coronary artery disease(s).

As used herein, an “amount effective” refers to an amount of the isolated fusion protein of the invention that is effective for treating and/or limiting coronary artery disease(s). The isolated fusion proteins are typically formulated as a pharmaceutical composition, such as those disclosed above, and can be administered via any suitable route, including orally, parentally, by inhalation spray, or topically in dosage unit formulations containing conventional pharmaceutically acceptable carriers, adjuvants, and vehicles. In a preferred embodiment, the pharmaceutical compositions and formulations are topically administration, such as in the form of ointments, lotions, creams, pastes, gels, drops, sprays, liquids and powders. Conventional pharmaceutical carriers, aqueous, powder or oily bases, thickeners and the like may be necessary or desirable.

Dosage regimens can be adjusted to provide the optimum desired response (e.g., a therapeutic or prophylactic response). A suitable dosage range may, for instance, be 0.1 ug/kg-100 mg/kg body weight; alternatively, it may be 0.5 ug/kg to 50 mg/kg; 1 ug/kg to 25 mg/kg, or 5 ug/kg to 10 mg/kg body weight. The isolated proteins can be delivered in a single bolus, or may be administered more than once (e.g., 2, 3, 4, 5, or more times) as determined by an attending physician.

The term “mammal” or “subject” or “patient” as used herein includes both humans and non-humans and include but is not limited to humans, non-human primates, canines, felines, murines, bovines, equines, and porcines.

In an eighth aspect, the invention comprises an isolated CD40 EC domain protein comprising one or more (e.g., 1, 2, 3, 4, 5, 6, 7, 8, 9, 10, 11, 12, 13, 14, 15, 16, 17, 18, 19, 20, 21, 22, 23, 24, or all 25) amino acid substitutions selected from the group consisting of: K46H, K46T, E64Y, E64S, E66T, D69Q, E74T, H76Q, K81S, K81H, K81T, K81R, P85Y, P85W, N86T, N86Q, Q93S, T112Y, T112S, T112K, E114N, E114R, A115V, E117Q and L121P relative to the wildtype human CD40 protein (SEQ ID NO: 40).

The term “CD40 EC domain” as used herein, refers to a domain within the human full length CD40 (SEQ ID NO: 40) from about amino acid 21 to about amino acid 277 of the human full length CD40 (for example SEQ ID NO: 260 comprises the CD40 EC domain), or C terminal truncations including, amino acid 21 to about amino acid 190 (or position 170, from about amino acid 21 to about amino acid 188 (or position 168 of the truncated, processed form shown in FIG. 13) or from about amino acid 21 to about amino acid 145 (or position 125 of the truncated, processed form shown in FIG. 13). The C-terminal truncations can be from about amino acid 21 to about amino acid 277, to about 250, about 200, about 190, about 180 about 170, about 160, about 150, about 140 or about 130 of the human full length CD40 (SEQ ID NO: 40). Additional examples of C-terminal truncated human CD40 EC domains are shown in FIG. 13. The human CD40 EC domain retains binding activity for human CD40 ligand (CD40L, or CD154) when truncated at several locations (e.g., residues 21-145 or 21-188 or 21-190, as shown in FIG. 13 where the numbering begins with the first amino acid of full length CD40, relative to wildtype full length human CD40). The CD40 EC domain and truncated variants (as shown in FIG. 13) of the invention can comprise a mutant human CD40 EC domain having one or more amino acid substitutions that increase the stability of the CD40 EC domain and/or increase binding affinity of CD40 EC domain to CD40 ligand. For example, the amino acid substitutions that increase the stability of the CD40 EC domain and/or increase binding affinity of CD40 EC domain to CD40 ligand could be at one or more amino acid residues selected from K46, E64, E66, D69, E74, H76, Q79, K81, D84, P85, N86, Q93, H110, T112, E114, A115, E117 or L121 relative to the wildtype human CD40 (these mutants can also be made in any of the C-terminal truncated CD40 EC variants disclosed here or shown in FIG. 13). Furthermore, the mutant human CD40 EC domain with increased stability and/or increased binding affinity can comprise one or more amino acid substitutions including but not limited to: K46H, K46T, E64Y, E64S, E66T, D69Q, E74T, H76Q, K81S, K81H, K81T, K81R, P85Y, P85W, N86T, N86Q, Q93S, T112Y, T112S, T112K, E114N, E114R, A115V, E117Q and L121P. In particular, the mutant human CD 40 EC domain can comprise one or more mutations: (i) E64S; (ii) E64Y; (iii) E66T; (iv) K81S; (v) K81T; (vi) T112Y; (vii) E64S and K81S; (viii) K81H and L121P; (ix) E114N and E117Q; (x) E64Y, K81T and P85Y; (xi) E64S, K81H and L121P; and (xii) E64Y, K81T and P85Y. The inventors have surprisingly discovered that the mutant human CD40 EC domain has improved properties of increased expression levels in in vitro systems and increased binding properties to CD40L. These novel mutant human CD40 EC domain proteins could be used as a method for treating an autoimmune disease or coronary artery disease/heart disease in a subject.

In one embodiment, the mutant human CD40 EC domain is fused with a human Fc domain. For example the Fc domain described here can be the wildtype or a mutated constant domain from IgG1, IgG2, IgG3 or IgG4 of human. For example, in one embodiment, the human IgG1 Fc domain of the invention comprises a hinge region, a CH2 domain and a CH3 domain. In another embodiment, the human IgG1 Fc domain can contain mutations of residues C220, C226, C229 in the hinge region (C to S) and mutations of residues P238 and P331 (P to S) in the CH2 domain. The Fc domain can be human IgG1 with the three cysteines of the hinge region (C220, C226, C229 relative to wildtype human IgG1) each changed to serine, and the proline at position 238 of the CH2 domain changed to serine, and the proline at position 331 of the CH2 domain changed to serine. In another embodiment, the Fc domain can be human IgG1 with N297 changed to any other amino acid, since mutating N297 eliminates the N-linked glycosylation site. In another embodiment, the Fc domain can be human IgG1 with one or more amino acid change between positions 292 and 300 relative to wildtype human IgG1. In another embodiment, the Fc domain can be human IgG1 with an amino acid addition or deletion at any position between residues 292 and 300 relative to wildtype human IgG1. In another embodiment, the Fc domain is human IgG1 with an SCC hinge (C220S, C226, C229); in a further embodiment an SSS (C220S, C226S, C229S) hinge. In further embodiments, the Fc domain can be human IgG1 with an SCC hinge and P238S/P331S mutations; with an SCC hinge and P238S/K322S/P331S mutations; an SSS hinge and P238S/P331S mutations; or an SSS hinge and P238S/K322S/P331S mutations. In another embodiment, the Fc domain can be human IgG1 with mutations that alter binding by Fc gamma receptors (I, II, III) without affecting FcRn binding important for half-life. These mutations are designed to prevent binding of C1q and prevent binding to Fc gamma receptors. The isolated protein fused to human IgG1 Fc retains the ability to bind to Protein A (useful for purification) and binding to FcRN (important for long serum half-life). The Fc portion of the human IgG1 heavy chain polypeptide has the ability to self-associate, a function which facilitates the formation of dimers.

In another embodiment the mutant human CD40 EC is modified to extend half-life of the CD40 EC domain. The polypeptides may be linked to other compounds to promote an increased half-life in vivo, such as by PEGylation, HESylation, PASylation, or glycosylation. Such linkage can be covalent or non-covalent as is understood by those of skill in the art. In another embodiment, the isolated proteins of the invention can be modified to extend half-life, such as by attaching at least one fusion protein to the isolated proteins for extending serum half-life, including but not limited to a polyethlyene glycol (PEG) group, serum albumin, transferrin, transferrin receptor or the transferrin-binding portion thereof, or combinations thereof. The isolated protein or fusion protein described herein can be stabilized in vivo and their half-life increased by binding to fusion proteins, such as PEG, which resist degradation and/or clearance or sequestration. The half-life of an isolated protein is increased if its functional activity persists, in vivo, for a longer period than a similar isolated protein which is not linked to a PEG polymer. Typically, the half-life of a PEGylated isolated protein is increased by 10%, 20%, 30%, 40%, 50% or more relative to a non-PEGylated isolated protein. Increases in the range of 2×, 3×, 4×, 5×, 10×, 20×, 30×, 40×, 50× or more of the half-life are possible. Alternatively, or in addition, increases in the range of up to 30×, 40×, 50×, 60×, 70×, 80×, 90×, 100×, 150× of the half-life are possible. According to the invention, a PEG-linked an isolated protein has a half-life of between 0.25 and 170 hours, preferably between 1 and 100 hours, more preferably between 30 and 100 hours, and still more preferably between 50 and 100 hours, and up to 170, 180, 190, and 200 hours or more. As use herein, the word “attached” refers to a covalently or non-covalently conjugated substance. The conjugation may be by genetic engineering or by chemical means.

As used herein, polyethylene glycol (PEG) is meant to encompass any of the forms of PEG that have been used to derivatize other proteins, such as mono-(C1-C10) alkoxy- or aryloxy-polyethylene glycol. More specifically, PEG can refer to a derivitized form of PEG, including, but not limited to N-hydroxylsuccinimide (NHS) active esters of PEG such as succinimidyl propionate, benzotriazole active esters, PEG derivatized with maleimide, vinyl sulfones, or thiol groups. Particular PEG formulations can include PEG-O—CH₂CH₂CH₂—CO₂—NHS; PEG-O—CH₂—NHS; PEG-O—CH₂CH₂—CO₂—NHS; PEG-S—CH₂CH₂—CO—NHS; PEG-O₂CNH—CH(R)—CO₂—NHS; PEG-NHCO—CH₂CH₂—CO—NHS; and PEG-O—CH₂—CO₂—NHS; where R is (CH₂)₄)NHCO₂(mPEG). PEG polymers useful in the invention may be linear fusion proteins, or may be branched wherein multiple PEG moieties are present in a single polymer.

Pegylation of the proteins of the invention can be carried out by any of the pegylation reactions known in the art. Pegylation can be carried out via an acylation reaction or an alkylation reaction with a reactive polyethylene glycol fusion protein (or an analogous reactive water-soluble polymer). For the acylation reactions, the polymer(s) selected should have a single reactive ester group. For reductive alkylation, the polymer(s) selected should have a single reactive aldehyde group. A reactive aldehyde is, for example, polyethylene glycol propionaldehyde, which is water stable, or mono C1-C10 alkoxy or aryloxy derivatives thereof. In general, chemical derivatization can be performed under any suitable conditions used to react a biologically active substance with an activated polymer fusion protein. Methods for preparing PEGylated proteins will generally comprise the steps of (a) reacting the protein with polyethylene glycol (such as a reactive ester, amine, aldehyde or maleimide derivative of PEG) under conditions whereby the protein becomes attached to one or more PEG groups, and (b) obtaining the reaction product(s). In general, the optimal reaction conditions for the acylation reactions will be determined based on known parameters and the desired result. For example, the larger the ratio of PEG:protein, the greater the percentage of poly-PEGylated product. In one embodiment, the protein will have a single PEG moiety at the amino terminus. In particular embodiments, the PEGylated protein provided by the invention has an average of about 1 to about 10, more particularly 2 to about 5 and more particularly 3 to 5 PEG fusion proteins covalently attached to each enzyme subunit in the composition.

In a ninth aspect, the invention comprises an isolated nucleic acid encoding the mutant CD40 EC domain isolated protein. The isolated nucleic acid sequence may comprise RNA or DNA. As used herein, “isolated nucleic acids” are those that have been removed from their normal surrounding nucleic acid sequences in the genome or in cDNA sequences. Such isolated nucleic acid sequences may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the invention.

In some embodiments, the novel nucleic acid can encode human CD40 EC domain fused in frame with the Fc domain of human IgG1, including, but not limited to, any of the Fc domains described herein. In some embodiments, the nucleic acids encode multispecific fusion proteins with multiple copies of the same domain (CD40 EC-CD40 EC) or different CD40 EC mutant domains linked via the Fc domain as a single chain construct. In yet another embodiment, the nucleic acid can encode multiple copies of the same or different mutant human CD40 EC domains in frame with or without an Fc domain. The Fc domain of the fusion protein facilitates high level expression from mammalian expression systems, and simplifies purification of fusion proteins by affinity chromatography. The selected Fc mutations reduce effector functions, including C1q and FcR binding, while maintaining a long half-life in vivo.

In a tenth aspect, the invention comprises a recombinant expression vector comprising the nucleic acid encoding the mutant CD40 EC domain isolated protein. The recombinant expression vector may comprise any embodiment or combination of embodiments disclosed above.

In an eleventh aspect, the invention comprises a host cell comprising the recombinant expression vector expressing CD40 EC domain isolated protein. The host cells can be either prokaryotic or eukaryotic. The cells can be transiently or stably transfected. Such transfection of expression vectors into prokaryotic and eukaryotic cells can be accomplished via any technique known in the art, including but not limited to standard bacterial transformations, calcium phosphate co-precipitation, electroporation, or liposome mediated-, DEAE dextran mediated-, polycationic mediated-, or viral mediated transfection. (See, for example, Molecular Cloning: A Laboratory Manual (Sambrook, et al., 1989, Cold Spring Harbor Laboratory Press; Culture of Animal Cells: A Manual of Basic Technique, 2nd Ed. (R. I. Freshney. 1987. Liss, Inc. New York, N.Y.).

In a twelfth aspect, the invention comprises a method for producing the mutant human CD40 EC isolated protein, comprising: (a) culturing the host cell expressing the mutant human CD40 EC domain under conditions suitable for expression of the nucleic-acid encoded protein; and (b) isolating the protein from the cultured cells. A method of producing a polypeptide according to the invention is an additional part of the invention and may comprise any embodiment or combination of embodiments disclosed above.

In a thirteenth aspect, the invention provides a pharmaceutical composition comprising the isolated mutant human CD40 EC domain protein of the invention and a pharmaceutically acceptable carrier. The pharmaceutical composition may comprise any embodiment or combination of embodiments disclosed above.

In a fourteenth aspect, the invention comprises a method for treating an autoimmune disease in a subject, comprising administering to a subject in need thereof the isolated mutant human CD40 EC domain of the invention or any of the pharmaceutical compositions of the invention, wherein the protein is administered in an amount effective to inhibiting an autoimmune response. The autoimmune disease can be selected from: systemic lupus erythematosis (Lupus or SLE), arthritis (collagen-induced arthritis), multiple sclerosis (experimental autoimmune encephalomyelitis, EAE), autoimmune thyroiditis (experimental autoimmune thyroiditis, EAT), colitis (hapten-induced colitis), atherosclerosis, coronary artery disease, allograft rejection and graft-versus-host disease.

As used herein, “treating an autoimmune disease” means accomplishing one or more of the following: (a) reducing the inflammation associated with the autoimmune disease(s) being treated; (b) limiting or preventing development of symptoms characteristic of the autoimmune disease(s) being treated; (c) inhibiting worsening of symptoms characteristic of the autoimmune disease(s) being treated; (d) limiting or preventing recurrence of the autoimmune disease(s) being treated in patients that have previously had the autoimmune disease(s); (e) limiting or preventing recurrence of symptoms in patients that were previously symptomatic for the autoimmune disease(s); and (f) limiting development of the autoimmune disease(s) in a subject at risk of developing the autoimmune disease(s), or not yet showing the clinical effects of the autoimmune disease(s).

As used herein, an “amount effective” refers to an amount of the isolated protein of the invention that is effective for treating and/or limiting autoimmune disease(s) and may comprise any embodiment or combination of embodiments disclosed above. The isolated proteins are typically formulated as a pharmaceutical composition, such as those disclosed above, and can be administered via any suitable route, including orally, parentally, by inhalation spray, or topically in dosage unit formulations containing conventional pharmaceutically acceptable carriers, adjuvants, and vehicles. In a preferred embodiment, the pharmaceutical compositions and formulations are topically administration, such as in the form of ointments, lotions, creams, pastes, gels, drops, sprays, liquids and powders. Conventional pharmaceutical carriers, aqueous, powder or oily bases, thickeners and the like may be necessary or desirable.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such variants necessarily have less than 100% sequence identity or similarity with the starting fusion protein. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant fusion protein.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

In one embodiment, a polypeptide of the invention consists of, consists essentially of, or comprises an amino acid sequence selected from the sequence listing and functionally active variants thereof. In an embodiment, a polypeptide includes an amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to an amino acid sequence set forth in the sequence listing. In an embodiment, a polypeptide includes a contiguous amino acid sequence at least 80%, 81%, 82%, 83%, 84%, 85%, 86%, 87%, 88%, 89%, 90%, 91%, 92%, 93%, 94%, 95%, 96%, 97%, 98%, or 99% identical to a contiguous amino acid sequence set forth in the sequence listing. In an embodiment, a polypeptide includes an amino acid sequence having at least 10, 15, 20, 25, 30, 35, 40, 45, 50, 55, 60, 65, 70, 75, 80, 85, 90, 95, 100, 200, 300, 400, or 500 (or any integer within these numbers) contiguous amino acids of an amino acid sequence set forth in the sequence listing.

The fusion proteins of the invention may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

In certain embodiments, the fusion proteins of the invention can employ one or more “linker domains,” such as polypeptide linkers. As used herein, the term “linker domain” refers to a sequence which connects two or more domains in a linear sequence. As used herein, the term “polypeptide linker” refers to a peptide or polypeptide sequence (e.g., a synthetic peptide or polypeptide sequence) which connects two or more domains in a linear amino acid sequence of a polypeptide chain. For example, polypeptide linkers may be used to connect a polypeptide domain to an Fc domain. Such polypeptide linkers can provide flexibility to the fusion proteins. In certain embodiments the polypeptide linker can be used to connect (e.g., genetically fuse) one or more Fc domains and/or one or more polypeptide domains. A fusion protein of the invention may comprise more than one linker domain or peptide linker.

As used herein, the term “gly-ser polypeptide linker” refers to a peptide that consists of glycine and serine residues. An exemplary gly/ser polypeptide linker comprises the amino acid sequence Ser(Gly₄Ser)n. In one embodiment, n=1. In one embodiment, n=2. In another embodiment, n=3, i.e., Ser(Gly₄Ser)3. In another embodiment, n=4, i.e., Ser(Gly₄Ser)4. In another embodiment, n=5. In yet another embodiment, n=6. In another embodiment, n=7. In yet another embodiment, n=8. In another embodiment, n=9. In yet another embodiment, n=10. Another exemplary gly/ser polypeptide linker comprises the amino acid sequence Ser(Gly₄Ser)n. In one embodiment, n=1. In one embodiment, n=2. In a preferred embodiment, n=3. In another embodiment, n=4. In another embodiment, n=5. In yet another embodiment, n=6.

As used herein, the terms “linked,” “fused”, or “fusion”, are used interchangeably. These terms refer to the joining together of two more elements or components or domains, by whatever means including chemical conjugation or recombinant means. Methods of chemical conjugation (e.g., using heterobifunctional crosslinking agents) are known in the art.

In certain embodiments, the isolated fusion proteins of the invention employ a polypeptide linker to join any two or more domains in frame in a single polypeptide chain. In one embodiment, the two or more domains may be independently selected from any of the Fc domains or polypeptide domains discussed herein. For example, in certain embodiments, a polypeptide linker can be used to fuse identical Fc domains, thereby forming a homomeric Fc region. In other embodiments, a polypeptide linker can be used to fuse different Fc domains (e.g. a wild-type Fc domain and a Fc domain variant), thereby forming a heteromeric Fc region. In other embodiments, a polypeptide linker of the invention can be used to genetically fuse the C-terminus of a first Fc domain (e.g. a hinge domain or portion thereof, a CH2 domain or portion thereof, a complete CH3 domain or portion thereof, a FcRn binding portion, an FcγR binding portion, a complement binding portion, or portion thereof) to the N-terminus of a second Fc domain (e.g., a complete Fc domain or fragment thereof).

In one embodiment, a polypeptide linker can comprise a portion of an Fc domain and polypeptide linkers typically precede or follow the Fc domain, or are inserted between functional domains. For example, in one embodiment, a polypeptide linker can comprise a portion of an immunoglobulin hinge domain of an IgG1, IgG2, IgG3, and/or IgG4 antibody. In another embodiment, a polypeptide linker can comprise a CH2 domain of an IgG1, IgG2, IgG3, and/or IgG4 antibody. In other embodiments, a polypeptide linker can comprise a CH3 domain of an IgG1, IgG2, IgG3, and/or IgG4 antibody. Other portions of an immunoglobulin (e.g. a human immunoglobulin) can be used as well. For example, a polypeptide linker can comprise a CH1 domain or portion thereof, a CL domain or portion thereof, a VH domain or portion thereof, or a VL domain or portion thereof. Said portions can be derived from any immunoglobulin, including, for example, an IgG1, IgG2, IgG3, and/or IgG4 antibody.

In some embodiments, a polypeptide linker can comprise at least a portion of an immunoglobulin hinge region. In one embodiment, a polypeptide linker comprises an upper hinge domain (e.g., an IgG1, an IgG2, an IgG3, or IgG4 upper hinge domain). In another embodiment, a polypeptide linker comprises a middle hinge domain (e.g., an IgG1, an IgG2, an IgG3, or an IgG4 middle hinge domain). In another embodiment, a polypeptide linker comprises a lower hinge domain (e.g., an IgG1, an IgG2, an IgG3, or an IgG4 lower hinge domain).

In other embodiments, polypeptide linkers can be constructed which combine hinge elements derived from the same or different antibody isotypes. In one embodiment, the polypeptide linker comprises a chimeric hinge comprising at least a portion of an IgG1 hinge region and at least a portion of an IgG2 hinge region. In one embodiment, the polypeptide linker comprises a chimeric hinge comprising at least a portion of an IgG1 hinge region and at least a portion of an IgG3 hinge region. In another embodiment, a polypeptide linker comprises a chimeric hinge comprising at least a portion of an IgG1 hinge region and at least a portion of an IgG4 hinge region. In one embodiment, the polypeptide linker comprises a chimeric hinge comprising at least a portion of an IgG2 hinge region and at least a portion of an IgG3 hinge region. In one embodiment, the polypeptide linker comprises a chimeric hinge comprising at least a portion of an IgG2 hinge region and at least a portion of an IgG4 hinge region. In one embodiment, the polypeptide linker comprises a chimeric hinge comprising at least a portion of an IgG1 hinge region, at least a portion of an IgG2 hinge region, and at least a portion of an IgG4 hinge region. In another embodiment, a polypeptide linker can comprise an IgG1 upper and middle hinge and a single IgG3 middle hinge repeat motif. In another embodiment, a polypeptide linker can comprise an IgG4 upper hinge, an IgG1 middle hinge and an IgG2 lower hinge.

In another embodiment, a polypeptide linker comprises or consists of a gly-ser linker. As used herein, the term “gly-ser linker” refers to a peptide that consists of glycine and serine residues. An exemplary gly/ser linker comprises an amino acid sequence of the formula (Gly₄Ser)ₙ, wherein n is a positive integer (e.g., 1, 2, 3, 4, or 5). A preferred gly/ser linker is (Gly₄Ser)₄. Another preferred gly/ser linker is (Gly₄Ser)₃. Another preferred gly/ser linker is (Gly₄Ser)₅. In certain embodiments, the gly-ser linker may be inserted between two other sequences of the polypeptide linker (e.g., any of the polypeptide linker sequences described herein). In other embodiments, a gly-ser linker is attached at one or both ends of another sequence of the polypeptide linker (e.g., any of the polypeptide linker sequences described herein). In yet other embodiments, two or more gly-ser linker are incorporated in series in a polypeptide linker. In one embodiment, a polypeptide linker of the invention comprises at least a portion of an upper hinge region (e.g., derived from an IgG1, IgG2, IgG3, or IgG4 fusion protein), at least a portion of a middle hinge region (e.g., derived from an IgG1, IgG2, IgG3, or IgG4 fusion protein) and a series of gly/ser amino acid residues (e.g., a gly/ser linker such as (Gly₄Ser)ₙ).

In one embodiment, a polypeptide linker of the invention comprises a non-naturally occurring immunoglobulin hinge region domain, e.g., a hinge region domain that is not naturally found in the polypeptide comprising the hinge region domain and/or a hinge region domain that has been altered so that it differs in amino acid sequence from a naturally occurring immunoglobulin hinge region domain. In one embodiment, mutations can be made to hinge region domains to make a polypeptide linker of the invention. In one embodiment, a polypeptide linker of the invention comprises a hinge domain which does not comprise a naturally occurring number of cysteines, i.e., the polypeptide linker comprises either fewer cysteines or a greater number of cysteines than a naturally occurring hinge fusion protein.

In other embodiments, a polypeptide linker of the invention comprises a polypeptide sequence which includes an —N linked glycosylation consensus sequence within the linker region. An exemplary NLG (N linked glycosylation containing linker) linker would incorporate a sequence such as VDGASSHVNVSSPSVQDI (SEQ ID NO: 58) or possibly DLVDGGSSTTSPVNVTSPSLE (SEQ ID NO: 56) between two functional domains of the fusion protein, such as the Fc and the extracellular domain of CD40, or the LDLR EGF-AB domain and the Fc domain. Other linker sequences or additions to linkers described herein which incorporate short additional sequences might be envisioned, including additional gly-ser repeats or hydrophilic residues for improving solubility.

In other embodiments, a polypeptide linker of the invention comprises a biologically relevant peptide sequence or a sequence portion thereof. For example, a biologically relevant peptide sequence may include, but is not limited to, sequences derived from an anti-rejection or anti-inflammatory peptide. Said anti-rejection or anti-inflammatory peptides may be selected from the group consisting of a cytokine inhibitory peptide, a cell adhesion inhibitory peptide, a thrombin inhibitory peptide, and a platelet inhibitory peptide. In a one preferred embodiment, a polypeptide linker comprises a peptide sequence selected from the group consisting of an IL-1 inhibitory or antagonist peptide sequence, an erythropoietin (EPO)-mimetic peptide sequence, a thrombopoietin (TPO)-mimetic peptide sequence, G-CSF mimetic peptide sequence, a TNF-antagonist peptide sequence, an integrin-binding peptide sequence, a selectin antagonist peptide sequence, an anti-pathogenic peptide sequence, a vasoactive intestinal peptide (VIP) mimetic peptide sequence, a calmodulin antagonist peptide sequence, a mast cell antagonist, a SH3 antagonist peptide sequence, an urokinase receptor (UKR) antagonist peptide sequence, a somatostatin or cortistatin mimetic peptide sequence, and a macrophage and/or T-cell inhibiting peptide sequence. Exemplary peptide sequences, any one of which may be employed as a polypeptide linker, are disclosed in U.S. Pat. No. 6,660,843, which is incorporated by reference herein.

It will be understood that variant forms of these exemplary polypeptide linkers can be created by introducing one or more nucleotide substitutions, additions or deletions into the nucleotide sequence encoding a polypeptide linker such that one or more amino acid substitutions, additions or deletions are introduced into the polypeptide linker. For example, mutations may be introduced by standard techniques, such as site-directed mutagenesis and PCR-mediated mutagenesis.

Polypeptide linkers of the invention are at least one amino acid in length and can be of varying lengths. In one embodiment, a polypeptide linker of the invention is from about 1 to about 50 amino acids in length. As used in this context, the term “about” indicates +/−two amino acid residues. Since linker length must be a positive integer, the length of from about 1 to about 50 amino acids in length, means a length of from 1 to 48-52 amino acids in length. In another embodiment, a polypeptide linker of the invention is from about 10-20 amino acids in length. In another embodiment, a polypeptide linker of the invention is from about 15 to about 50 amino acids in length.

In another embodiment, a polypeptide linker of the invention is from about 20 to about 45 amino acids in length. In another embodiment, a polypeptide linker of the invention is from about 15 to about 25 amino acids in length. In another embodiment, a polypeptide linker of the invention is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more amino acids in length.

Polypeptide linkers can be introduced into polypeptide sequences using techniques known in the art. Modifications can be confirmed by DNA sequence analysis. Plasmid DNA can be used to transform host cells for stable production of the polypeptides produced.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP® and BLASTN® or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST® algorithm, which is described in Altschul et al., J. Mol. Biol. 215:403-410 (1990). Software for performing BLAST® analyses is publicly available through the National Center for Biotechnology Information website.

### EXEMPLARY ASPECTS

Below are examples of specific aspects for carrying out the present invention. The examples are offered for illustrative purposes only, and are not intended to limit the scope of the present invention in any way. Efforts have been made to ensure accuracy with respect to numbers used (e.g., amounts, temperatures, and the like), but some experimental error and deviation should, of course, be allowed for.

### Example 1

### Construction of LDLR EGF-AB Ig Fusion Genes

Human low-density lipoprotein receptor epidermal growth factor-like repeat AB domain (LDLR EGF-AB) was amplified from total cDNA prepared from human liver RNA (Ambion/Applied Biosystems, Austin, Tex.). Sequence specific 5′ and 3′ primers used were from the published sequences. The sequence of the clone was verified by sequencing analysis. The genetic database accession number for the human LDLR, transcript variant 2, is NCBI NM_{—}001195798.1, and the Genebank accession number for the human LDLR mRNA is AY114155.1. The associated full-length protein sequence is AAM56036.1 (SEQ ID NO: 02).

To create an amino terminal LDLR EGF-AB-Ig fusion protein, primers were designed to create a fusion gene with the human VK3LP and the mouse IgG2a or human IgG1 Fc domains. A 5′ primer was generated that would attach an AgeI site to the amino terminus of LDLR EGF-AB domain, permitting fusion of this region of the LDLR to the human VK3LP for expression as a component of a secreted fusion protein

(SEQ ID NO. 5: hLDLR-egfab5′ age: 5′-accggtggaggg
accaacgaatgcttggacaacaac-3′).

The 3′ primer was an antisense primer that attached a restriction site to the carboxyl end of the LDLDR EGF-AB domain of LDLR compatible with fusion to the immunoglobulin, with or without an intervening linker domain such as (gly4ser)ₓ. The sequence of the region and primer is SEQ ID NO. 6: hLDLR-egfab3′bgl/xho (can be inserted with or without (gly4ser)ₙ linker(s)

SEQ ID NO: 06:
5′-ctcgagatctgagcccacagccttgcaggccttcgtgtg-3.

Construction of mutant form(s) of human LDLR EGF-AB domain: To make an H306Y mutant form of LDLR EGF-AB-Ig to increase the binding avidity for PCSK9, overlapping oligonucleotides were designed which introduce the new tyrosine residue and replace the wild type histidine at this amino acid.

Sense oligo: SEQ ID NO 7: EGFAB-H306Y-S:
5′-ggctgttcctacgtctgcaatg-3′
Antisense Oligo: SEQ D NO. 8: EGFAB-H306Y-AS:
5′-cattgcagacgtaggaacagcc-3′

Similarly, in constructing an -Ig fusion protein encoding LDLR-EGF-AB domains with the insert at the carboxy terminus, oligonucleotides were designed to fuse the EGF-AB domain to the carboxyl end of the -Ig tail followed by a linker domain that includes an N-linked glycosylation site. The sequence of the primers is listed below:

SEQ ID NO 9: hLDLR-egfab3′BE:
5′-cccgggaccaacgaatgcttggacaacaac-3′

(SmaI creates a blunt end to ligate to the EcoRV after the NLG linker—there are internal EcoRV sites in EGFAB)

SEQ ID NO. 10: hLDLR-egfab3-stop
5′-tctagattatcatgagcccacagccttgcaggccttcgtgtg-3′

### Example 2

### Construction of the Human LDLR EGF-AB-hIgG1 Fusion Gene

Human LDLR EGF-AB (SEQ ID NO. 3) was isolated by PCR amplification from human liver total RNA obtained from Ambion/Applied Biosystems (Austin, Tex.). Two microgram (2 μg) of total RNA was used as template to prepare cDNA by reverse transcription. The RNA, 300 ng random primers, and 500 ng Oligo dT (12-18), and 1 ul 25 mM dNTPs were combined and denatured at 80° C. for 5 minutes prior to addition of enzyme. SUPERSCRIPT® III reverse transcriptase (Invitrogen,/Life Technologies, Carlsbad, Calif.) was added to the RNA plus primer mixture in a total volume of 25 μl in the presence of second strand buffer and 0.1 M DTT provided with the enzyme. The reverse transcription reaction was allowed to proceed at 50° C. for one hour. Reactions were further purified by QIAQUICK® PCR purification columns, and cDNA eluted in 40 microliters EB buffer prior to use in PCR reactions. Two microliters cDNA eluate were added to PCR reactions containing 50 pmol 5′ and 3′ primers specific for human LDLR EGF-AB, and 45 microliters of PCR high fidelity SuperMix (Life Technologies, Grand Island, N.Y.) was added to 0.2 ml PCR reaction tubes. PCR reactions were performed using a C1000 thermal cycler (BioRad, Hercules Calif.). Reactions included an initial denaturation step at 95° C. for 2 minutes, followed by 34 cycles with a 94° C., 30 sec denaturation, 50° C., 30 sec annealing, and 68° C., 1 minute extension step, followed by a final 4 minute extension at 72° C. Once wild type sequences for the LDLR EGF-AB domain region of LDLR were isolated, the fragments were TOPO cloned into pCR2.1 vectors, DNA prepared using the QIAGEN spin plasmid miniprep kits according to manufacturer's instructions. Plasmid DNA was sequenced using ABI Dye Terminator v3.1 ready reaction mix according to manufacturer's instructions. Sequencing reactions were performed using the BIG DYE® Terminator Ready Sequencing Mix v3.1 (PE-Applied Biosystems, Foster City, Calif.) according to the manufacturer's instructions. Samples were subsequently purified using Autoseq G25 columns (GE Healthcare) and the eluates dried in a Savant vacuum dryer, denatured in Template Suppression Reagent (PE-ABI), and analyzed on an ABI 310 Genetic Analyzer (PE-Applied Biosystems). The sequence was edited, translated, and analyzed using Vector NTI® version 11.5 (Informax/Invitrogen, North Bethesda, Md.).

### Example 3

### Isolation of CD40 Extracellular Domains for Construction of Soluble CD40L Binding Fusion Proteins

Human CD40 full length cDNA (NCBI reference sequence number: NM_{—}001250.4, human CD40 transcript variant 1) was isolated by PCR amplification from human PBMC total RNA obtained from a normal healthy human blood donor. Two microgram (2 μg) of total RNA was used as template to prepare cDNA by reverse transcription. The RNA, 300 ng random primers, and 500 ng Oligo dT (12-18), and 1 ul 25 mM dNTPs were combined and denatured at 80° C. for 5 minutes prior to addition of enzyme. SUPERSCRIPT® III reverse transcriptase (Invitrogen,/Life Technologies, Carlsbad, Calif.) was added to the RNA plus primer mixture in a total volume of 25 μl in the presence of second strand buffer and 0.1 M DTT provided with the enzyme. The reverse transcription reaction was allowed to proceed at 50° C. for one hour. Reactions were further purified by QIAQUICK® PCR purification columns, and cDNA eluted in 40 microliters EB buffer prior to use in PCR reactions. Two microliters cDNA eluate were added to PCR reactions containing 50 pmol 5′ and 3′ primers specific for human CD40, and 45 microliters of PCR high fidelity SuperMix (Life Technologies, Grand Island, N.Y.) was added to 0.2 ml PCR reaction tubes. PCR reactions were performed using a C1000 thermal cycler (BioRad, Hercules Calif.). Reactions included an initial denaturation step at 95° C. for 2 minutes, followed by 34 cycles with a 94° C., 30 sec denaturation, 50° C., 30 sec annealing, and 68° C., 1 minute extension step, followed by a final 4 minute extension at 72° C. Once wild type sequences for the full length and extracellular domain regions of CD40 were isolated, the fragments were TOPO cloned into pCR2.1 vectors, DNA prepared using the QIAGEN spin plasmid miniprep kits according to manufacturer's instructions. Plasmid DNA was sequenced using ABI Dye Terminator v3.1 ready reaction mix according to manufacturer's instructions. Sequencing reactions were performed using the BIG DYE® Terminator Ready Sequencing Mix v3.1 (PE-Applied Biosystems, Foster City, Calif.) according to the manufacturer's instructions. Samples were subsequently purified using Autoseq G25 columns (GE Healthcare) and the eluates dried in a Savant vacuum dryer, denatured in Template Suppression Reagent (PE-ABI), and analyzed on an ABI 310 Genetic Analyzer (PE-Applied Biosystems). The sequence was edited, translated, and analyzed using Vector NTI® version 11.5 (Informax/Invitrogen, North Bethesda, Md.). To clone full length CD40 (SEQ ID NO. 39), the following primers were used with random primed cDNA as template:

SEQ ID NO. 37: hCD40-FL-5 (48 mer):
5′-gttaagcttgccaccatggttcgtctgcctctgcagtgcgtcctct
gg-3′
SEQ ID NO. 38: hCD40-FL-3′ (48 mer):
5′-tctagattatcactgtctctcctgcactgagatgcgactctctttg
cc-3′

Plasmids encoding the CD40 full length clone were used as template to clone the extracellular domain truncated forms of CD40. The following primers were used for cloning CD40 extracellular domain fusions between the signal peptide domain and the linker and/or -Ig tail domain:

SEQ ID NO. 41: hCD40-5age (48 mer):
5′-accggtgaaccacccactgcatgcagagaaaaacagtacctaataa
ac-3′
SEQ ID NO. 42: hCD40-3D3 (41 mer):
5′-ctcgagatctggctcgcagatggtatcagaaacccctgtag-3′
SEQ ID NO. 43: hCD40-3D4s (44 mer):
5′-ctcgagatctggaccacagacaacatcagtcttgtttgtgcctg-3′
SEQ ID NO. 44: hCD40-3D4L (40 mer)
5′-ctcgagatctgaatcctggggaccacagacaacatcagtc-3′

The first primer, hCD40-5age (SEQ ID NO 41), was used in each PCR amplification to attach the CD40 extracellular domain at the AgeI site present at the end of the human VK3 leader peptide cassette. Each of the hCD40-3 primers, hCD40-3D3 (SEQ ID NO. 42), hCD40-3D4s (SEQ ID NO. 43), and hCD40-3D4L (SEQ ID NO 44), were used with this 5′ primer in order to create different CD40 extracellular domain cassettes with varying endpoints in domains 3 or 4 of CD40, as diagrammed in FIG. 13.

### Example 4

### Isolation of Human and Mouse-Ig Tails and Introduction of Desired Mutations into the Coding Sequence

For isolation of mouse and human-Ig tails, RNA was derived from mouse or human tissue as follows. A single cell suspension was generated from mouse spleen in RPMI culture media. Alternatively, human PBMCs were isolated from fresh, whole blood using Lymphocyte Separation Media (LSM) Organon Teknika (Durham, N.C.), buffy coats harvested according to manufacturer's directions, and cells washed three times in PBS prior to use. Cells were pelleted by centrifugation from the culture medium, and 2×10⁷ cells were used to prepare RNA. RNA was isolated from the cells using the QIAGEN RNEASY® kit (Valencia, Calif.) total RNA isolation kit and QIAGEN QIASHREDDER® columns according to the manufacturer's instructions accompanying the kits. One microgram (4 μg) of total RNA was used as template to prepare cDNA by reverse transcription. The RNA, 300 ng random primers, and 500 ng Oligo dT (12-18), and 1 μl 25 mM dNTPs were combined and denatured at 80° C. for 5 minutes prior to addition of enzyme. SUPERSCRIPT® III reverse transcriptase (Invitrogen, Life Technologies) was added to the RNA plus primer mixture in a total volume of 25 μl in the presence of second strand buffer and 0.1 M DTT provided with the enzyme. The reverse transcription reaction was allowed to proceed at 50° C. for one hour. cDNA was purified using QIAquick (QIAGEN) PCR purification columns according to manufacturer's directions, and eluted in 40 microliters EB buffer prior to use in PCR reactions.

Wild type mouse or human-Ig tails were isolated by PCR amplification using the cDNA described above as template. The following primers listed as part of the sequence listing were used for initial amplification of wild type sequences, but incorporated the desired mutational changes in the hinge domain:

SEQ ID NO. 11: mahIgG1CH2M: 47 mer
5′-tctccaccgtctccagcacctgaactcctgggtggatcgtcagtct
tcc-3′
SEQ ID NO 12: hIgG1-5sss: 49 mer
5′-agatctcgagcccaaatcttctgacaaaactcacacatctccaccg
tct-3′
SEQ ID NO 13: mahIgG1S: 51 mer
5′-tctagattatcatttacccggagagagagagaggctcttctgcgtg
tagtg-3′

PCR reactions were performed using a C1000 thermal cycler (BioRad, Hercules Calif.). Reactions included an initial denaturation step at 95° C. for 2 minutes, followed by 34 cycles with a 94° C., 30 sec denaturation, 50° C., 30 sec annealing, and 72° C., 1 minute extension step, followed by a final 4 minute extension at 72° C. Once wild type tails were isolated, the fragments were TOPO cloned into pCR2.1 vectors, DNA prepared using the QIAGEN spin plasmid miniprep kits according to manufacturer's instructions and clones sequenced using ABI Dye Terminator v3.1 sequencing reactions according to manufacturer's instructions.

DNA from the correct clones was used as template in overlap extension PCRs to introduce mutations at the desired positions in the coding sequence for human-IgG1. PCR reactions were set up using the full length wild type clones as template (1 microliter), 50 pmol 5′ and 3′ primers to PCR each portion of the -Ig tail up to and including the desired mutation site from each direction, and PCR High Fidelity SuperMix (Invitrogen, Carlsbad Calif.), in 50 microliter reaction volumes using a short amplification cycle. As an example of the overlapping PCR mutagenesis, the primer combination used to introduce the P331S mutation into human-IgG1, was as follows:

A 5′ subfragment was amplified using the full-
length wild type clone as template, and the 5′
primer was:
SEQ ID NO 14: hIgG1-5scc:
5′agatctcgagcccaaatcttctgacaaaactcacacatgtccaccgt
gt-3′,
or
SEQ ID NO 12: hIgG1-5sss:
5′-agatctcgagcccaaatcttctgacaaaactcacacatctccaccg
tct-3′
while the 3′ primer was:
SEQ ID NO 15: P331AS:
5′-gttttctcgatggaggctgggagggctttgttggagacc-3′.
A 3′ subfragment was amplified using the full-
length wild type clone as template and the 5′
primer was:
SEQ ID NO 16: P331S:
5′ aaggtctccaacaaagccctcccagcctccatcgagaaaacaatct
cc-3′,
while the 3′ primer was:
SEQ ID NO 13: mahIgG1S:
5′-tctagattatcatttacccggagagagagagaggctcttctgcgtg
tagtg-3′.

Once subfragments were amplified and isolated by agarose gel electrophoresis, they were purified by QIAQUICK® gel purification columns and eluted in 30 microliters EB buffer according to manufacturer's instructions. Two rounds of PCR were then performed with the two subfragments as overlapping templates in new reactions. The cycler was paused and the 5′ (SEQ ID NO 14: hIgG1-5scc or SEQ ID NO 12: hIgG1-5sss, see above) and 3′ (SEQ ID NO 13: mahIgG1S, see above) flanking primers were added to the reactions (50 pmol each). PCR amplifications were then carried out for 34 cycles at the conditions described for the wild type fusion proteins above. Full length fragments were isolated by gel electrophoresis, and TOPO cloned into pCR2.1 vectors for sequence analysis. Fragments from clones with the correct sequence were then subcloned into expression vectors for creation of the different fusion genes described herein.

### Example 5

### Expression of Fusion Proteins in a Transient COS7 Transfection System

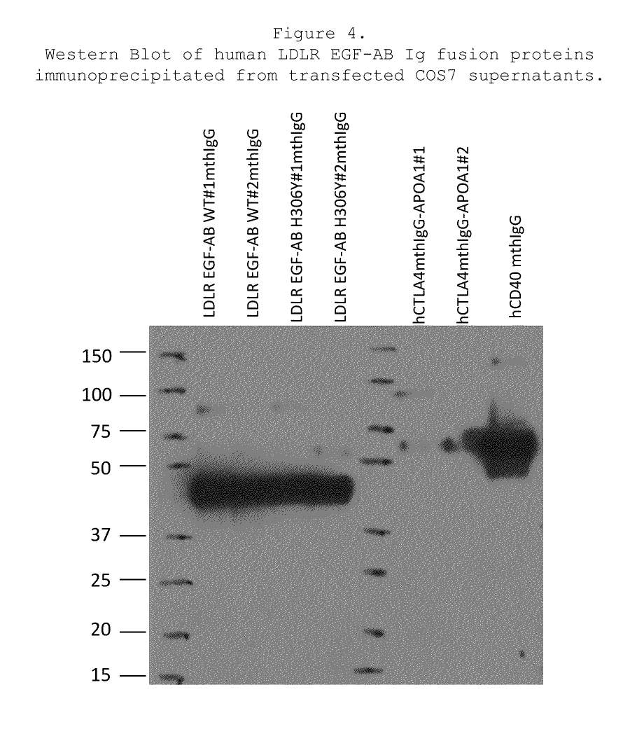

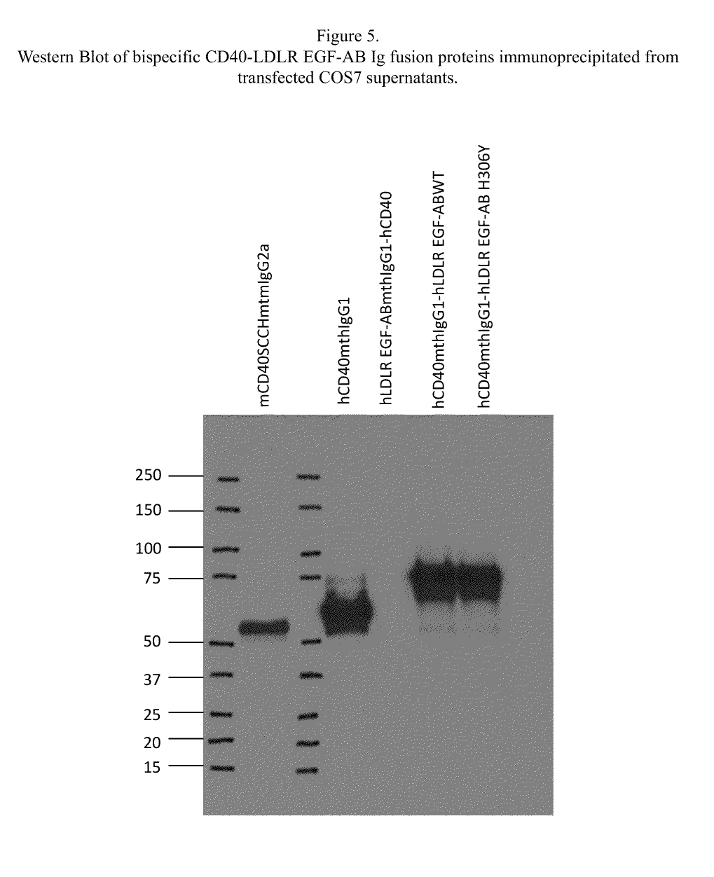

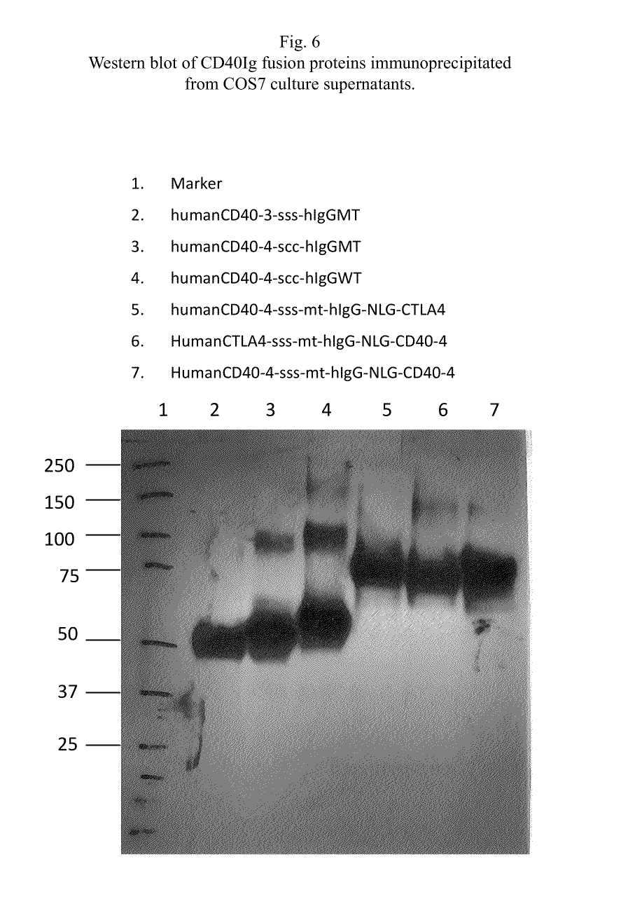

This example illustrates transfection and expression of fusion proteins described herein in a mammalian transient transfection system. The -Ig fusion gene fragments with correct sequence were inserted into the mammalian expression vector pDG, and DNA from positive clones was amplified using QIAGEN plasmid preparation kits (QIAGEN, Valencia, Calif.). Mini-plasmid preparations (2.5 ug DNA for 60 mm plates) were used for COS7 transfections using the QIAGEN POLYFECT® reagent and following the manufacturer's instructions. Culture supernatants were harvested 48-72 hours after transfection. Protein A agarose (IPA 400HC, Catalog #10-2500-03, Repligen, Waltham, Miss.) (100 ul) was used to immunoprecipitate 0.5-1.0 ml culture supernatants, 4° C., overnight. Immuneprecipitates were washed and LDS Life Technologies, Grand Island, N.Y.) sample buffer added to the protein A agarose. For reducing gels, sample reducing agent was added (1/10 final volume). Samples were heated at 72° C. for 10 minutes, and protein A beads centrifuged prior to loading samples on NuPAGE® 4-12% Bis-Tris gels. Gels were subjected to electrophoresis in MOPS buffer at 175 volts for 1-1.5 hours, and proteins transferred to nitrocellulose. Blots were blocked overnight at 4° C. in 5% nonfat milk. Blots were incubated with 1:2500 dilution of horseradish peroxidase conjugated goat anti-human IgG (or anti-mouse IgG) from Jackson Immunoresearch. Blots were washed three times for 30 minutes each, and developed in Thermo Scientific ECL reagent for 5 minutes followed by a 40 second exposure to autoradiography film. FIGS. 4-6 show Western Blot analysis of protein A immunoprecipitates from representative COS7 transient transfections of several of the described preferred embodiments. Proteins separated by electrophoresis on each reducing gel were transferred to nitrocellulose using the X Cell mini-blot apparatus (Catalog # EI002, Life Technologies, Grand Island, N.Y.) at 30 mAmp for 1.5 hours. Western blots were blocked in 5% nonfat milk in PBS, 4° C., overnight with agitation. Blots were probed with 1:3000 HRP conjugated goat anti-human IgG (or anti-mouse IgG, Jackson Immunoresearch, West Grove, Pa.) in PBS, incubated for 2 hours at room temperature. Blots were washed 3 times for 30 minutes each in PBS/0.05% Tween 20, and were developed in ThermoScientific ECL reagent for 5 minutes. Blots were exposed to autoradiograph film for 30 seconds to 2 minutes, depending on the blot. Positive and negative controls were included in each transfection series. Transfected samples are as indicated in the figures.

### Example 6

### Expression of LDLR EGF-AB mthIgG and LDLR EGF-AB H306Y mthIgG, Multi-SubunitIg Fusion Constructs and Fusion Proteins in Stable CHO Cell Lines

This example illustrates expression of the different -Ig fusion genes described herein in eukaryotic cell lines and characterization of the expressed fusion proteins by SDS-PAGE and by IgG sandwich ELISA.

The -Ig fusion gene fragments with correct sequence were inserted into the mammalian expression vector pDG, and DNA from positive clones was amplified using QIAGEN plasmid preparation kits (QIAGEN, Valencia, Calif.). The recombinant plasmid DNA (200 μg) was then linearized in a nonessential region by digestion with AscI, purified by phenol extraction, and resuspended in tissue culture media, Excell 302 (Catalog #14324, SAFC). Cells for transfection, CHO DG44 cells, were kept in logarithmic growth, and 2×10⁷ cells harvested for each transfection reaction. Linearized DNA was added to the CHO cells in a total volume of 0.8 ml for electroporation.

Stable production of the -Ig fusion protein was achieved by electroporation of a selectable, amplifiable plasmid, pDG, containing the LDLR EGF-AB-mthIgG cDNA under the control of the CMV promoter, into Chinese Hamster Ovary (CHO) CHO DG44 cells. The pDG vector is a modified version of pcDNA3 encoding the DHFR selectable marker with an attenuated promoter to increase selection pressure for the plasmid. Plasmid DNA was prepared using QIAGEN maxiprep kits, and purified plasmid was linearized at a unique AscI site prior to phenol extraction and ethanol precipitation. Salmon sperm DNA (Sigma-Aldrich, St. Louis, Mo.) was added as carrier DNA, and 200 μg each of plasmid and carrier DNA was used to transfect 2×10⁷ CHO DG44 cells by electroporation. Cells were grown to logarithmic phase in Excell 302 media (Catalog #13424C, SAFC Biosciences, St. Louis, Mo.) containing glutamine (4 mM), pyruvate, recombinant insulin, penicillin-streptomycin, and 2×DMEM nonessential amino acids (all from Life Technologies, Grand Island, N.Y.), hereafter referred to as “Excell 302 complete” media. Media for untransfected cells also contained HT (diluted from a 100× solution of hypoxanthine and thymidine) (Invitrogen/Life Technologies). Media for transfections under selection contained varying levels of methotrexate (Sigma-Aldrich) as selective agent, ranging from 50 nM to 1 μM. Electroporations were performed at 280 volts, 950 microFarads. Transfected cells were allowed to recover overnight in non-selective media prior to selective plating in 96 well flat bottom plates (Costar) at varying serial dilutions ranging from 500 cells/well to 4000 cells/well. Culture media for cell cloning was Excell 302 complete, containing 50 nM methotrexate. Once clonal outgrowth was sufficient, serial dilutions of culture supernatants from master wells were screened for expression of -Ig fusion protein by use of an -IgG sandwich ELISA. Briefly, NUNC MAXISORP™ plates were coated overnight at 4° C. with 2 microgram/ml F(ab′2) goat anti-human IgG (Jackson Immunoresearch, West Grove, Pa.) in PBS. Plates were blocked in PBS/2% BSA, and serial dilutions of culture supernatants incubated at room temperature for 2-3 hours. Plates were washed three times in PBS/0.05% Tween 20, and incubated with horseradish peroxidase conjugated F(ab′2) goat anti-human IgG (Jackson Immunoresearch, West Grove, Pa.) at 1:7500 in PBS/0.5% BSA, for 1-2 hours at room temperature. Plates were washed four times in PBS/0.05% Tween 20, and binding detected with SureBlue Reserve, TMB substrate (KPL Labs, Gaithersburg, Md.). Reactions were stopped by addition of equal volume of 1N HCl, and plates read at 450 nM on a SYNERGY® 2 plate reader (Biotek, Winooski, Vt.). The clones with the highest production of the fusion protein were expanded into T25 and then T75 flasks to provide adequate numbers of cells for freezing and for scaling up production of the fusion protein. Production levels were further increased in cultures from the four best clones by progressive amplification in methotrexate containing culture media. At each successive passage of cells, the Excell 302 complete media contained an increased concentration of methotrexate, such that only the cells that amplified the DHFR plasmid could survive.

Supernatants were collected from CHO cells expressing the LDLR EGF-AB-mthIgG1, filtered through 0.2 μm PES express filters (Nalgene, Rochester, N.Y.) and were passed over a Protein A-agarose (IPA 300 crosslinked agarose, or IPA 400HC crosslinked agarose) column (Repligen, Waltham, Mass.). The column was conditioned with 0.1M citrate buffer, pH2.2, then supernatant adjusted to pH 8.0 with 0.5M NHCO3, and loaded by gravity flow to allow binding of fusion protein, then washed with column wash buffer (90 mM Tris-Base, 150 mM NaCl, 0.05% sodium azide, pH 8.7) or Dulbecco's modified PBS, pH 7.4 prior to elution. Bound protein was eluted using 0.1 M citrate buffer, pH 3.2. Fractions (0.8-0.9 ml) were collected into 0.2 ml 0.5M NaCO3 to neutralize, and protein concentration of each fraction was determined at 280 nM using a NANODROP® (Wilmington Del.) microsample spectrophotometer, with blank determination using 0.1 M citrate buffer, pH 3.2, 0.5M NaCO3 at a 10:1 v:v ratio. Fractions containing fusion protein were pooled, and buffer exchange performed by dialysis against D-PBS(Hyclone, ThermoFisher Scientific, Dallas, Tex.) pH 7.4. After dialysis, protein was filtered using 0.1 uM filter units, and aliquots tested for endotoxin contamination using Pyrotell LAL gel clot system single test vials (STV) (Catalog # G2006, Associates of Cape Cod, East Falmouth, Mass.). An extinction coefficient that corresponds to the OD 280 of a 1 mg/ml solution of protein was determined to be 1.05 for WT and 1.07 for H306Y, using the protein analysis tools in the Vector NTI® Version 11.5 Software package (Informax, North Bethesda, Md.) and the predicted cleavage site from the online ExPasy protein analysis tools.

### Example 7

### SDS-PAGE Analysis of LDLR EGF-AB Ig Fusion Protein

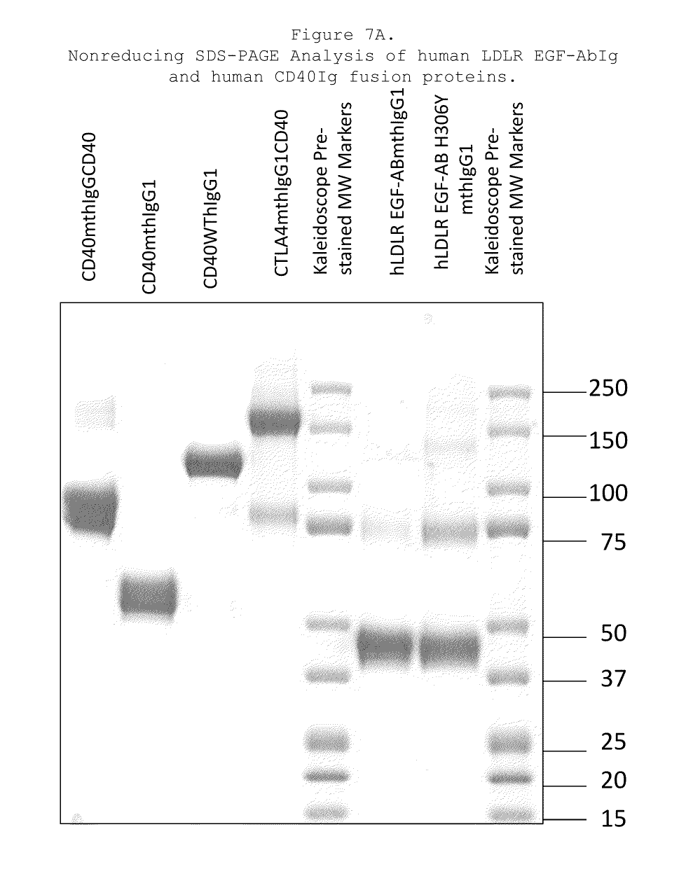

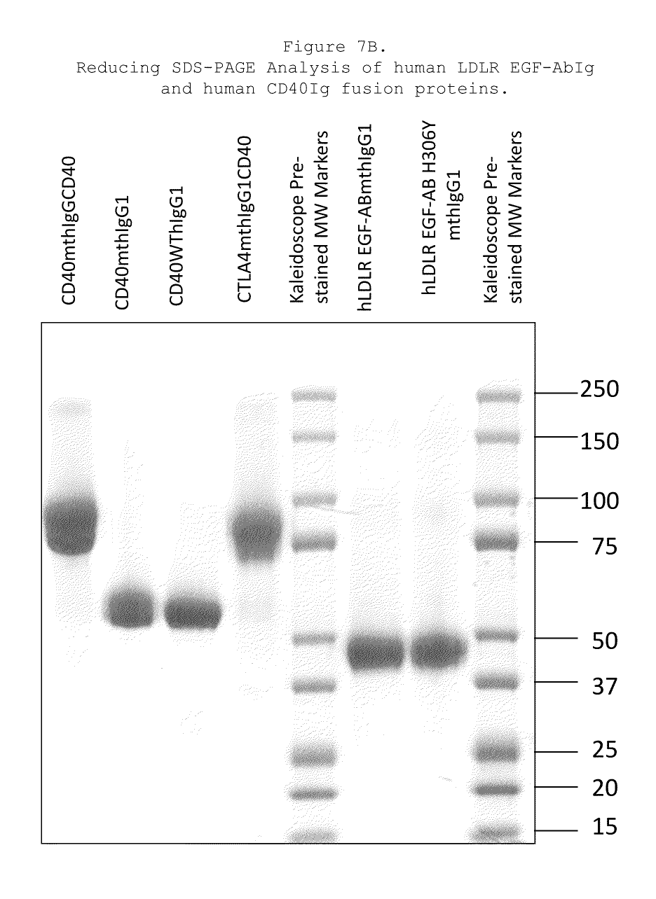

Purified LDLR EGF-AB-Ig was analyzed by electrophoresis on SDS-Polyacrylamide (NuPAGE®) gels. Fusion protein samples were heated at 72° C. for 10 minutes in LDS sample buffer with and without reduction of disulfide bonds and applied to 5-12% BIS-Tris gels (Catalog #NP0301, LIFEsciences, Grand Island, N.Y.). Five micrograms of each purified protein was loaded on the gels. The proteins were visualized after electrophoresis by Coomassie Blue staining (Pierce Gel Code Blue Stain Reagent, Catalog #24590, Pierce, Rockford, Ill.), and destaining in distilled water. Molecular weight markers were included on the same gel (Kaleidoscope Prestained Standards, Catalog #161-0324, Bio-Rad, Hercules, Calif.). The results from a representative nonreducing gel are shown in FIG. 7A. Lanes are as follows from left to right: Lane #1: hCD40EC-SSSH-mthIgG-NLG-CD40EC, Lane#2: human (h)CD40-SSSH-mthIgG1, Lane #3: hCD40EC-SCCH-WThIgG1, Lane #4: hCTLA4-SSSH-mthIgG1-NLG-hCD40EC, Lane#5: Kaleidoscope Prestained MW markers, Lane #6: hLDLR EGF-AB WT-SSSH-mthIgG1, Lane #7: hLDLR DGF-AB H306Y-SSSH-mthIgG1, Lane #8: Kaleidoscope prestained MW markers. Approximate molecular weights are indicated on the figures. Similarly, FIG. 7B shows a representative reducing gel analyzing the same samples and loaded in the same order.

### Example 8

### Use of a PCSK9 Antigen Binding ELISA to Assess Binding of LDLR Fusion Proteins

An antigen binding ELISA was performed to assess the ability of immobilized LDL receptor domain fusion protein to bind to PCSK9 antigen in solution. The 96-well plate (NUNC MAXISORP®, ThermoFisher Scientific) was coated with serial dilutions of 2.0, 1.0, and 0.5 μg/ml of LDLR EGF-ABIg fusion protein or control CD40Ig fusion protein (at 2 ug/ml) overnight. After washing three times with PBS containing 0.05% Tween, the plate was blocked with PBS/2% BSA at 4° C. overnight. The plate was washed three times and then incubated with PCSK9-his6 antigen (AcroBiosystems, Gaithersburg Md., Catalog # PC9-H5223) serially diluted (2× increments) across the plate from 10 μg/ml to 10 ng/ml. Plates were incubated at room temperature for 2 hours. Plates were washed four times prior to addition of detection antibody, an HRP (horseradish peroxidase) conjugated anti-his6 tag antibody (Catalog # R93125, Life Technologies, Grand Island, N.Y.), at 1:2000 for 1.5 hour at room temperature. Plates were washed 4 times, then Sureblue Reserve TMB substrate (Catalog #: 53-00-02, KPL, Gaithersburg, Md.) was added to the plate at 80 μl/well. Development was stopped by addition of 80 μl/well 1N HCl. Samples were read at 450 nm using a SYNERGY®® 2 Biotek plate reader (Biotek Instruments, Winooski, Vt.) and data analyzed using Gen 5.2 software.

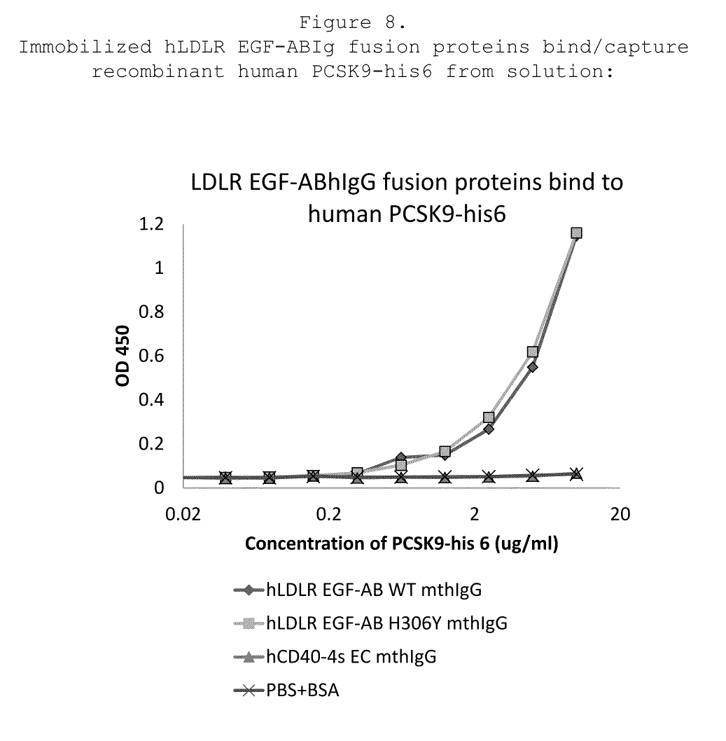

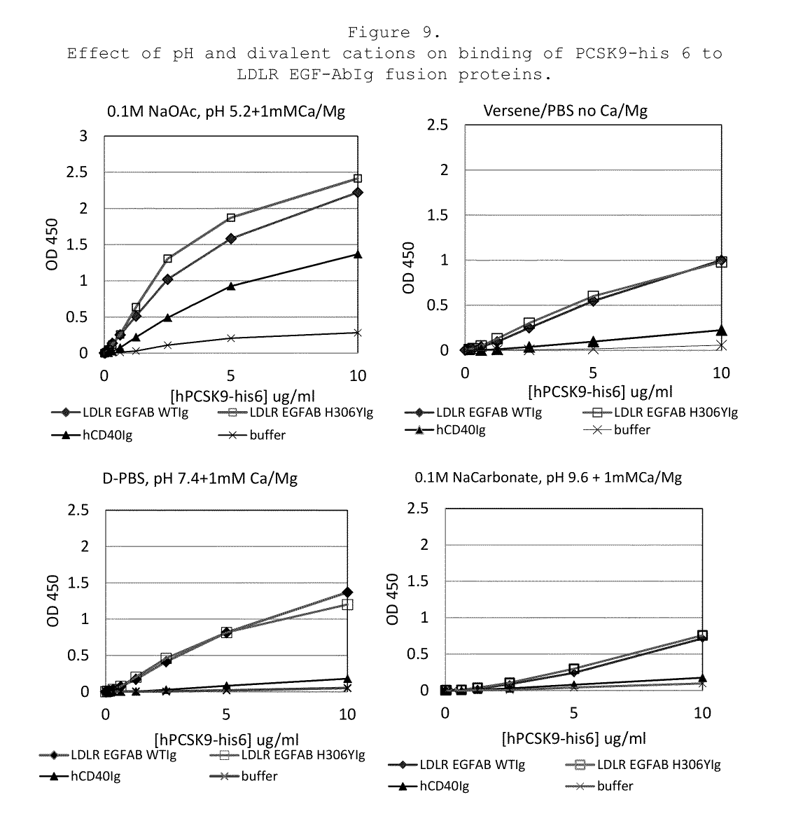

FIG. 8 shows the results from a representative PCSK9 binding ELISA with LDLR EGF-ABIg wild type and H306Y fusion proteins, and a non-binding CD40IgG fusion protein as negative control, each added to the wells of the plate at 2 ug/ml. FIG. 9 shows the results from a similar ELISA where the effects of divalent cations and pH were explored by including Ca++ and Mg++ in the binding steps at pH 5.2, 7.4, or 9.6, or by binding in the presence of Versene to chelate any available divalent cations from the binding steps.

### Example 9

### Immobilized PCSK9 can Capture LDLR EGF-ABhIgG Fusion Proteins from Solution

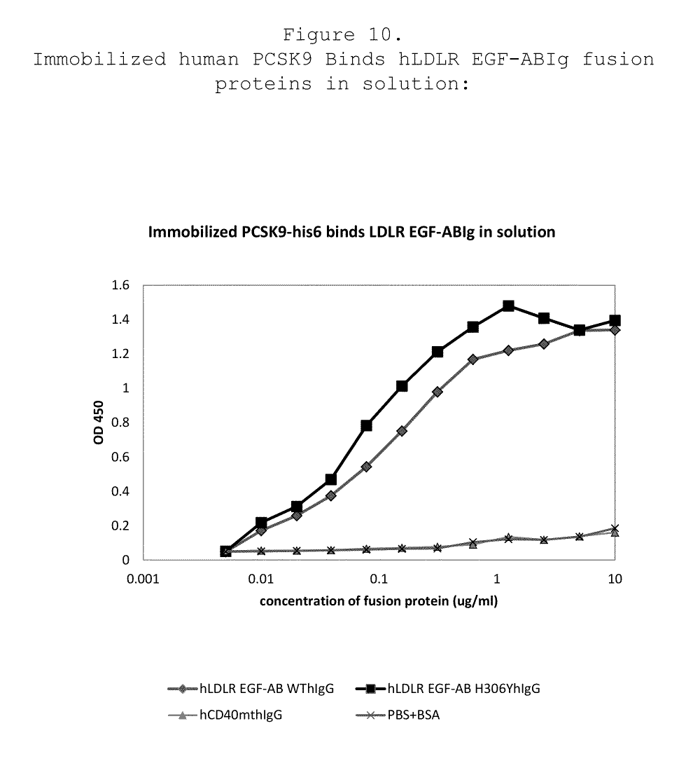

An antigen binding ELISA was performed in the opposite direction in order to assess whether immobilized or soluble phase protein exhibited altered binding properties for PCSK9-LDLR interactions. Briefly, ELISA plates were coated with 2 μg/ml capture antibody in 50 microliters, anti-his6 tag antibody, (Catalog #652502, Biolegend, San Diego Calif.), and incubated overnight at 4° C. Plates were washed with PBS/0.05% Tween 20, blocked in PBS/2% BSA for 2 hours at room temperature, washed with PBS/0.05% Tween-20, and incubated with 2 μg/ml PCSK9-his 6 antigen (Catalog # PC9-H5223, AcroBiosystems, Bethesda, Md.), and incubated overnight at 4° C. Plates were washed and incubated with serial dilutions of LDLR EGF-AB-IgG fusion protein or with control fusion proteins (hCD40IgG), at 4° C., overnight. Plates were washed four times, and incubated with 1:7500 goat anti-human IgG detection antibody (Catalog #109-036-003, Jackson Immunoresearch, West Grove, Pa.) conjugated with horseradish peroxidase. Plates were washed five times in PBS/0.05% Tween-20, and incubated with 80 microliters of TMB substrate. Reactions were stopped by addition of 80 μl 1N HCl, and samples read at 450 nm on a SYNERGY® 2 Biotek plate reader (Biotek Instruments, Winooski, Vt.). Raw data was analyzed with Gen5 v2 software. The results are shown in FIG. 10.

### Example 10

### Immobilized LDLR EGF-AB-Ig Fusion Proteins Bind to and Capture Mouse PCSK9 Protein

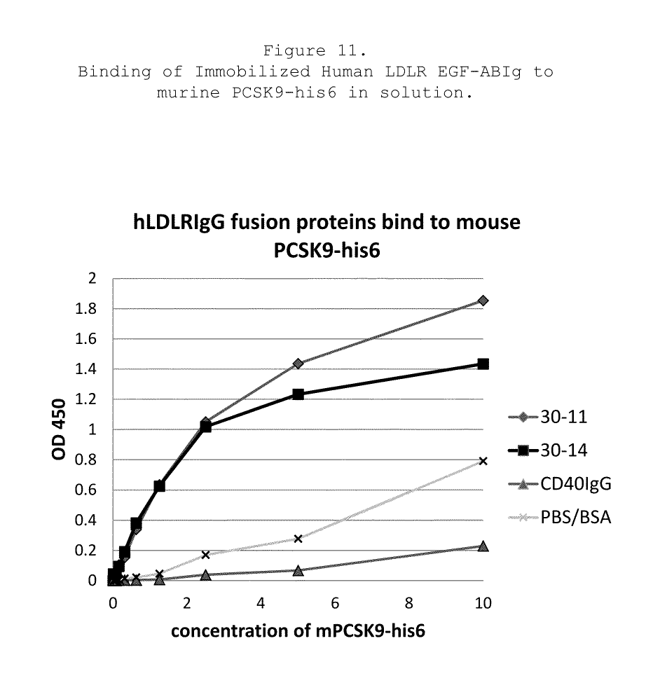

It was important to assess whether the human LDLR EGF-ABmthIgG fusion proteins were able to bind to mouse PCSK9 in order to study their functional effects on cholesterol levels in vitro or in vivo using mouse models for human disease. An antigen binding ELISA was performed using immobilized human LDLR EGF-ABmthIgG fusion proteins at several concentrations (2.0, 1.0, and 0.5 ug/ml) or hCD40mthIgG (at 2 ug/ml). Proteins were immobilized in 0.1 M carbonate buffer (pH 9.6) at 4° C., overnight. Plates were then washed twice and blocked in PBS/2% BSA, 200 ul per well, overnight, 4° C. Plates were washed twice, and then serial dilutions of mouse PCSK9-his6 (Sino Biologicals, Beijing, China, Cat#0.50251-M08H) protein was added to each well, starting at 10 ug/ml and diluting in two fold serial increments across the plate to a final dilution of 10 ng/ml. Plates were incubated 2 hours at room temperature, washed four times, and incubated for 1.5 hours with HRP-anti his6 (Life Technologies, Grand Island, N.Y., Catalog #R93125) at a 1:2000 dilution. Plates were washed 5 times, prior to addition of 1 component TMB substrate (KPL Inc., Gaithersburg, Md., Catalog #52-00-02), 80 ul/well. Reactions were stopped by addition of 80 ul 1N HCl, and plates analyzed using SYNERGY® 2 plate reader as described previously. The results are shown in FIG. 11, and demonstrate that human LDLR EGF-AB fusion proteins are able to bind to and capture mouse PCSK9 fusion protein from solution.

### Example 11

### LDLR EGF-AB Ig Fusion Proteins were Assessed for their Ability to Lower Cholesterol In Vivo Using a Mouse Model

FIG. 12 shows results from an in vivo assay exploring the effect of the LDLR EGF-AB WT Ig fusion protein on LDL cholesterol levels in ldlr (+/−) mice. Mice were treated by intraperitoneal injection with one dose of 10 mg/kg of each protein. Blood was drawn at day 4 and day 7 and analyzed for cholesterol levels. Mice treated with the LDLR-EGF-AB fusion protein showed significant reduction in LDL cholesterol levels at day 4 compared to the baseline (p<0.05) and to the control at day 4 (p<0.001). For LDLR-EGF-AB WT Ig mice, n=9, and for the control group, n=3 mice

### Example 12

### The Human CD40 Extracellular Region can be Truncated at Various Positions in Domains 3 and 4 to Create CD40L Binding Fusion Proteins

As described previously, the CD40 extracellular domain was subcloned using three different truncation endpoints in domains 3 to 4. The predicted peptide sequence for three different transcript variants of human CD40 are shown in FIG. 13, and the truncation endpoints and their designation are shown with arrows and identifiers at the appropriate locations. Similarly, the mouse CD40 can be truncated at similar sites, and the truncation endpoints for mouse CD40 are diagrammed in FIG. 14.

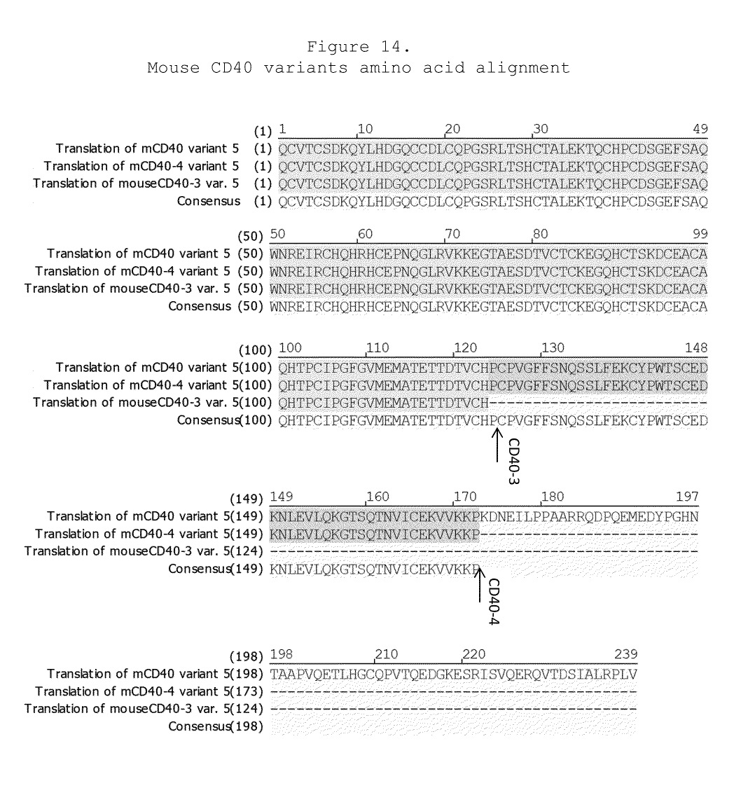

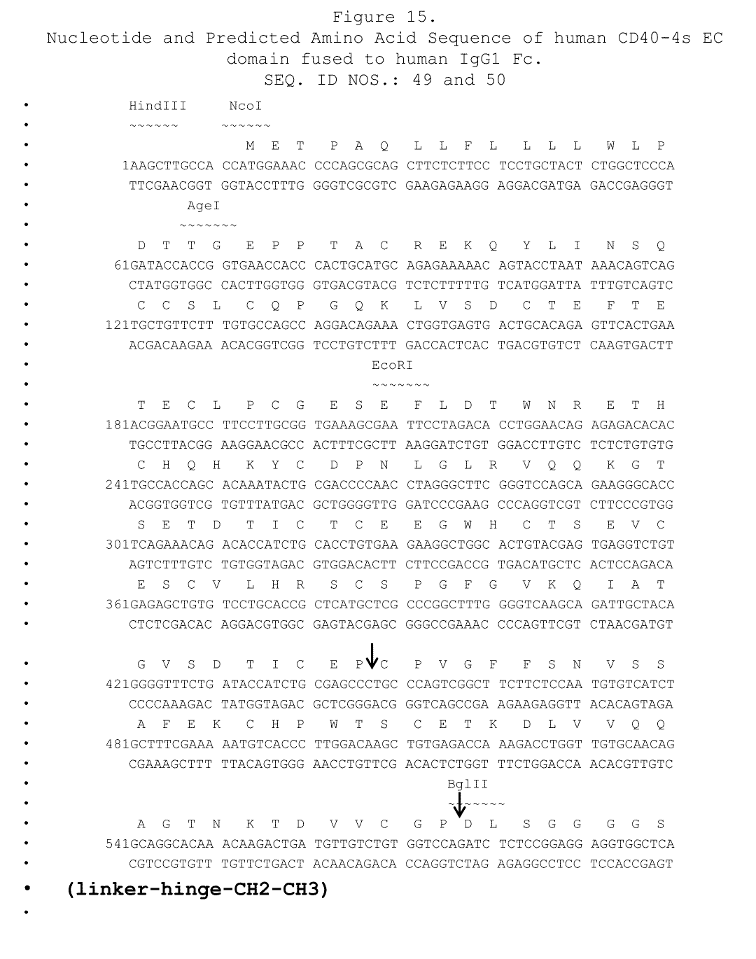

One of these CD40 extracellular fusion genes exhibited better binding to CD40L and better expression in transient transfections of COS7 cells. This construct was designated hCD40-4s EC, and the predicted nucleotide and amino acid sequence for the -Ig fusion gene are shown in FIG. 15.

### Example 13

### CD40Ig Fusion Proteins Bind to CD40L Expressed on Human Jurkat Cells

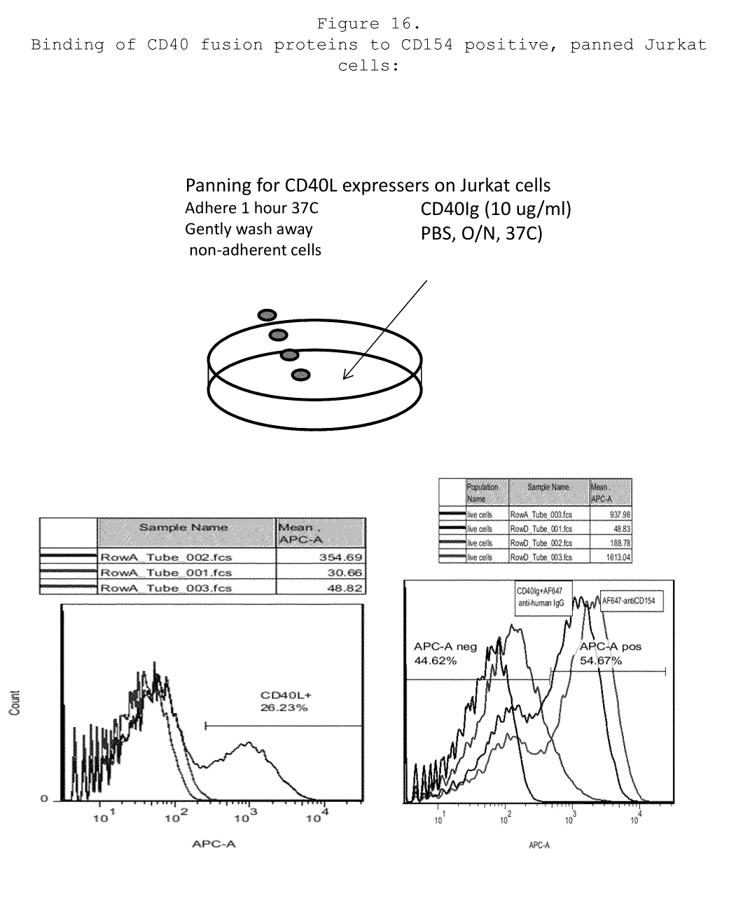

Some clones of human Jurkat cells constitutively express human CD40 ligand (CD40L). The cell line available in our lab was heterogeneous, with some of the population constitutively expressing detectable CD40L and some expressing little or no CD40L on the cell surface. Cells expressing high CD40L were selected by panning with immobilized anti-CD40L. Briefly, flasks or dishes were coated with CD40L antibody at 10 ug/ml in PBS overnight at 37° C. Buffer was removed and plates washed with RPMI/10% FBS, and incubated in media until cells were harvested. Cells were harvested from culture by centrifugation and re-plated in PBS/2% BSA for one hour at 37° C. Plates were gently rocked to remove non-adhered cells and the liquid aspirated using a sterile aspirator. Plates were washed twice with PBS, and then the remaining cells re-suspended in RPMI/10% FBS. Cells were cultured several days and an aliquot harvested to screen by flow cytometry for CD40L expression. FIG. 16 shows that panning of Jurkat cells resulted in a higher percentage of cells with constitutive expression of CD40L on their surface. FIG. 16 also shows that one of the CD40 EC-Ig fusion proteins bound to panned Jurkat cells at levels comparable to a directly conjugated anti-CD154/CD40L antibody.

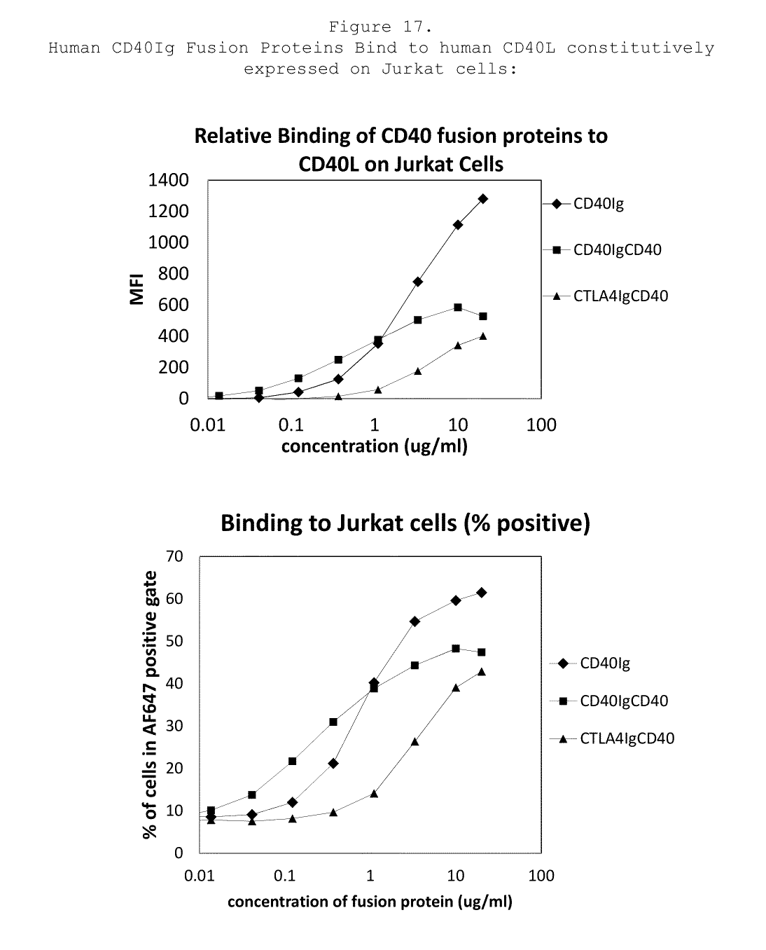

FIG. 17 shows the relative binding to Jurkat cells of serial dilutions of purified CD40 Ig fusion proteins. The binding titrations show that all of the CD40Ig fusion proteins exhibit binding, and that CD40 can be expressed at either the amino or carboxyl end of an -Ig fusion protein and still bind to CD40L. CD40 expressed at the carboxyl end of the -Ig fusion protein apparently binds with a lower affinity than if expressed at the amino terminal end of the protein as indicated by the lower MFI at saturation for these fusion proteins. The CD40×CD40 fusion protein showed lower MFI than the CD40Ig fusion proteins; however, for every -Ig tail bound by the anti-human IgG fluorescent conjugate, there should be two binding sites for human CD40L. For the CTLA4-Ig-CD40 fusion protein, only a single CD40 binding motif is present for every -Ig tail, and the CTLA4 end does not bind to Jurkat cells since they do not show constitutive expression of CD80 or CD86.

### Example 14

### CD40Ig Fusion Proteins Block Binding of Labeled CD40Ig to CD40L on Jurkat Cells to Different Extents

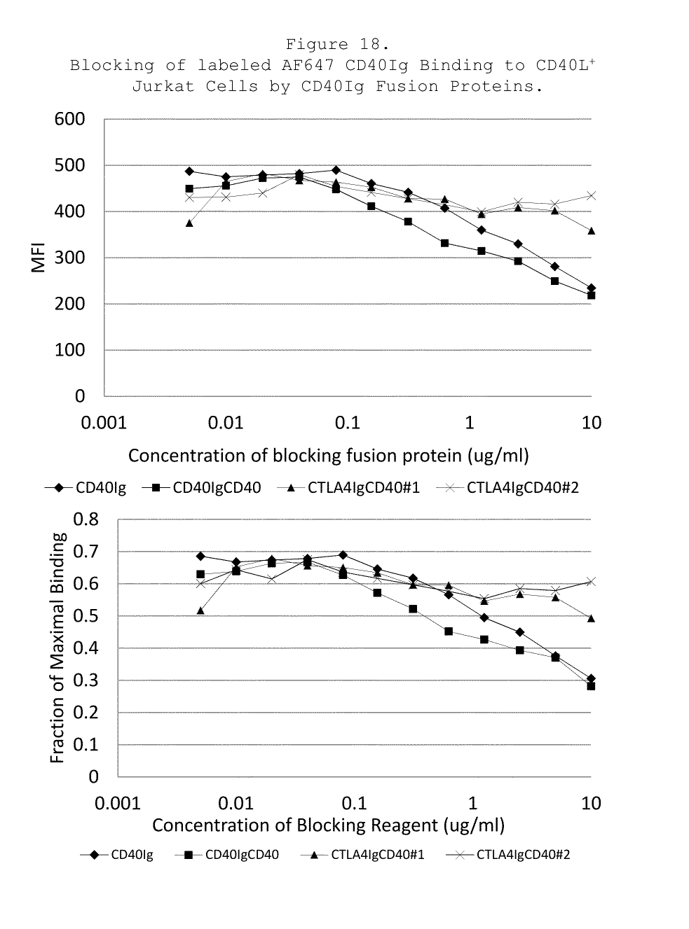

The CD40-4s EC-mthIgG fusion protein was conjugated to Alexa Fluor 647 (AF647-CD40IgG) in order to explore how well the different fusion proteins block its binding to panned Jurkat cells. FIG. 18 shows results from a blocking assay where Jurkat cells were incubated with serial dilutions of each purified fusion protein in FACS staining buffer (PBS/3% FBS), incubated for one hour on ice, and then AF647-CD40Ig added to the binding reaction. Samples were incubated on ice for another 60 minutes, washed three times, and re-suspended in staining buffer. Samples were analyzed by flow cytometry on a FACs CANTO machine, and gated on live cells. FCS files were analyzed using FlowJo software, and the mean fluorescence compared at each dilution of fusion protein. The data are graphed in FIG. 18 and show that although the binding intensity of the CD40×CD40 fusion protein was less in the direct binding assay shown in FIG. 17, at concentrations between 0.1-5 ug/ml, it showed higher blocking activity as measured by decreased binding of the AF647-CD40Ig.

### Example 15

### CD40Ig Fusion Proteins Bind to Immobilized Recombinant Human CD40L

An antigen binding ELISA was developed to assess whether the CD40 fusion proteins could bind with high affinity to immobilized recombinant human CD40L in vitro. Briefly, ELISA plates were coated with 2 μg/ml recombinant human CD40L in 60 ul/well, (Catalog #310-02, Peprotech, Rocky Hill, N.J.), and incubated overnight at 4° C. Plates were washed twice with PBS/0.05% Tween 20, blocked in 200 ul/well PBS/2% BSA for 2 hours at room temperature, washed with PBS/0.05% Tween-20, and incubated with serial dilutions of purified fusion protein or culture supernatants from CD40 EC domain containing proteins stably or transiently transfected into mammalian cells. Samples were incubated for a minimum of 2 hours, washed three times in PBS/0.05% Tween-20, and incubated with 1:7500 goat anti-human IgG detection antibody (Catalog #109-036-003, Jackson Immunoresearch, West Grove, Pa.) for 1.5 hours at room temperature. ELISAs were developed with 80 microliters of TMB substrate (KPL). Reactions were stopped by addition of 80 μl 1N HCl, and samples read at 450 nm on a SYNERGY® 2 Biotek plate reader (Biotek Instruments, Winooski, Vt.). Raw data was analyzed with Gen5 v2 software. The CD40L binding results for purified CD40 EC domain-Ig fusion proteins are shown in FIG. 19, and in this assay, CD40×CD40 fusion proteins showed the highest level of binding to immobilized CD40L.

### Example 16

### Human CD40Ig Fusion Proteins do not Mediate Platelet Activation or Aggregation In Vitro

Because one of the biggest obstacles to development of CD40 or CD40L binding fusion proteins has been the occurrence of thrombotic events due to platelet activation through FcR and CD40-CD40L stimulation, it was important to assess whether novel CD40 inhibitors mediate platelet activation. Platelet aggregation in vitro is one measure of platelet activation that if it occurs in vivo, can lead to serious thrombotic events and death of treated patients. A microplate kinetic platelet aggregation assay was developed with the SYNERGY® 2 plate reader that permits comparison of new fusion proteins described herein with platelet activation agents for aggregation in vitro.

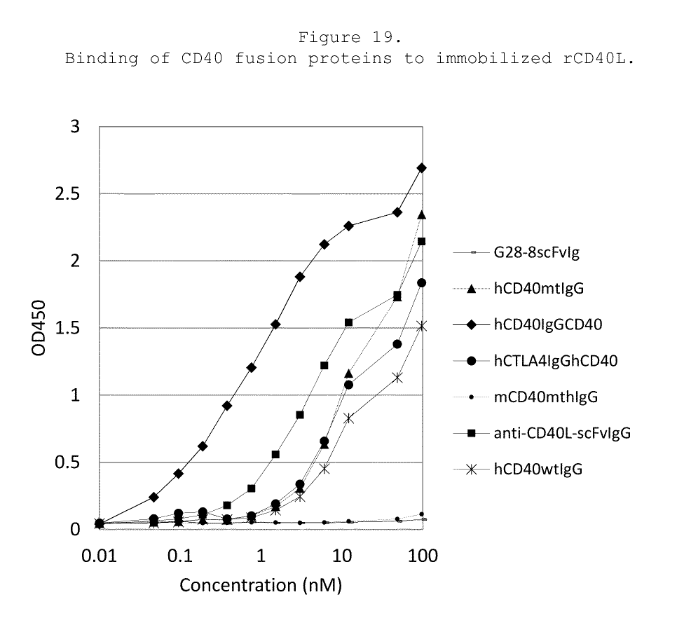

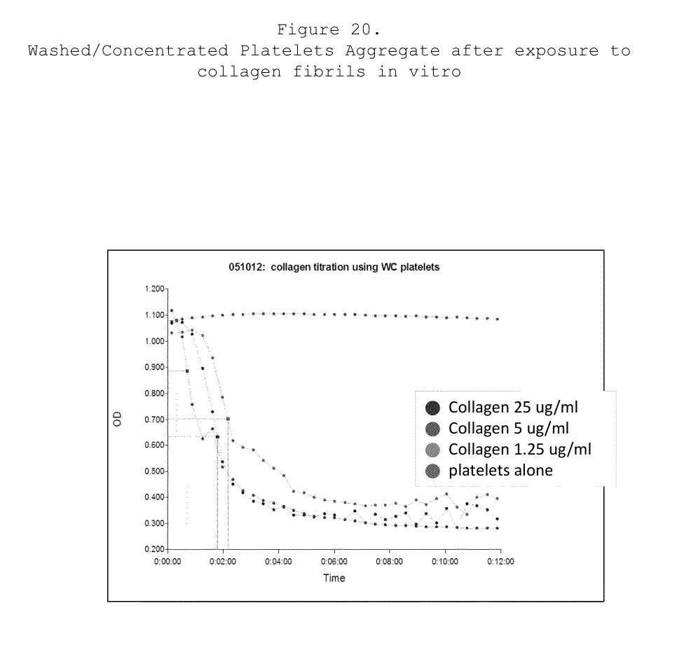

Human whole blood from normal healthy donors was isolated by venipuncture into citrate containing blood collection tubes. Alternatively, 50 mL of blood were drawn into a syringe containing 3 mL of 4% (w/v) sodium citrate. For 50 ml blood, 7 mL of acid citrate dextrose (ACD: 2.5% (w/v) sodium citrate, 2% (w/v) glucose and 1.5% (w/v) citric acid) was added prior to separation of the whole blood to prevent platelet activation. The citrate containing blood was centrifuged without braking at 300 g for 20 minutes, and platelet rich plasma harvested to new tubes. Plate rich plasma was centrifuged at 1500 g for 10-15 minutes to harvest platelets. Platelet poor plasma was harvested from the tubes and saved for other assays. Platelet pellets were resuspended in 1.5 ml modified Tyrodes-HEPES buffer (134 mM NaCl, 2.9 mM KCl, 0.34 mM Na2HPO4, 12 mM NaHCO3, 1 mM MgCl2, 20 mM HEPES and 5 mM glucose, pH 7.3) and rested prior to assay. The SYNERGY® 2 plate reader was programmed with a kinetic protocol at 37° C. with constant linear agitation at maximum speed, and kinetic absorbance reads performed at 30 second intervals for 25-40 minutes to monitor aggregation. Platelets in 75 ul were added to each well of a 96-well plate and a pre-read was performed for 5 minutes to be sure that the platelets were not aggregated prior to addition of test reagents. Reagents to be tested were diluted to the appropriate concentration (2× final), in modified Tyrodes-HEPES buffer containing 3.6 mM CaCl2. The final concentration in test wells would therefore be 1.8 mM CaCl2. Initial experiments tested various platelet isolation and concentration methods, assay buffers, and mode of agitation for effects on platelet aggregation. Collagen, native collagen fibrils from equine tendons (Chrono Par, P/N 00385, Chrono-LOG Corporation, Broomall, Pa.) was used as a positive control for platelet aggregation in all assays. FIG. 20 shows collagen mediated platelet aggregation trace results of a representative assay performed during the assay development process. It was determined that optimal collagen mediated platelet aggregation could be observed at 5 ug/ml collagen.

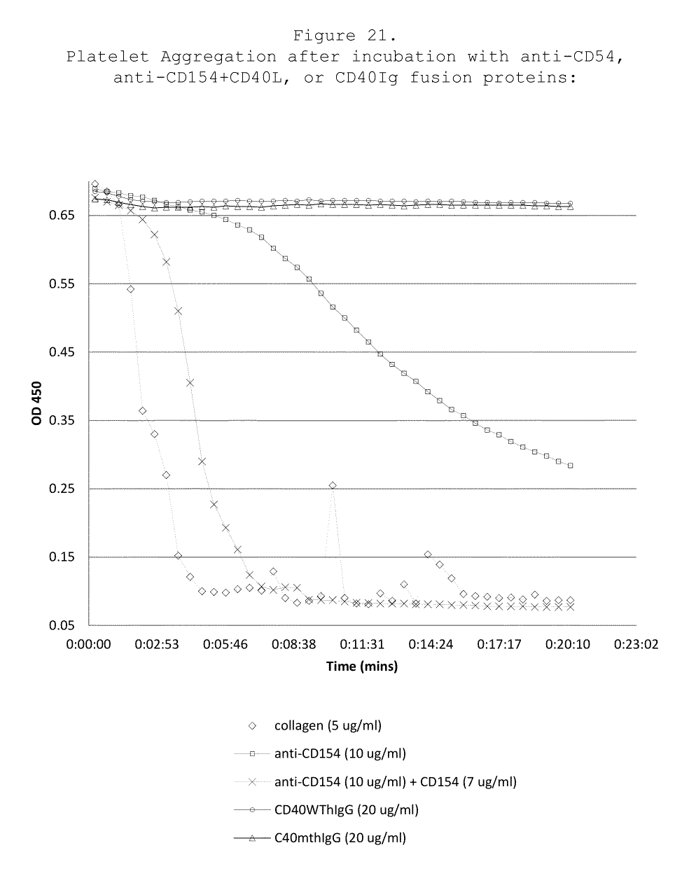

FIG. 21 shows results of a platelet aggregation assay where CD40-Ig fusion proteins were compared to collagen fibril mediated aggregation and to a monoclonal antibody targeted to human CD154 (CD40L), or to a preformed immune complex between anti-CD154 (Biolegend, San Diego, Calif.) and human recombinant CD40L (Peprotech, Rocky Hill, N.J.). None of the CD40 containing fusion proteins mediated platelet aggregation, while the CD 154 antibody mediated somewhat inefficient platelet aggregation. In the presence of recombinant human CD154/CD40L, the antibody mediated platelet aggregation was greatly increased.

### Example 17

### Human CD40Ig Fusion Proteins in Complex with CD40L do not Mediate Platelet Activation or Aggregation In Vitro

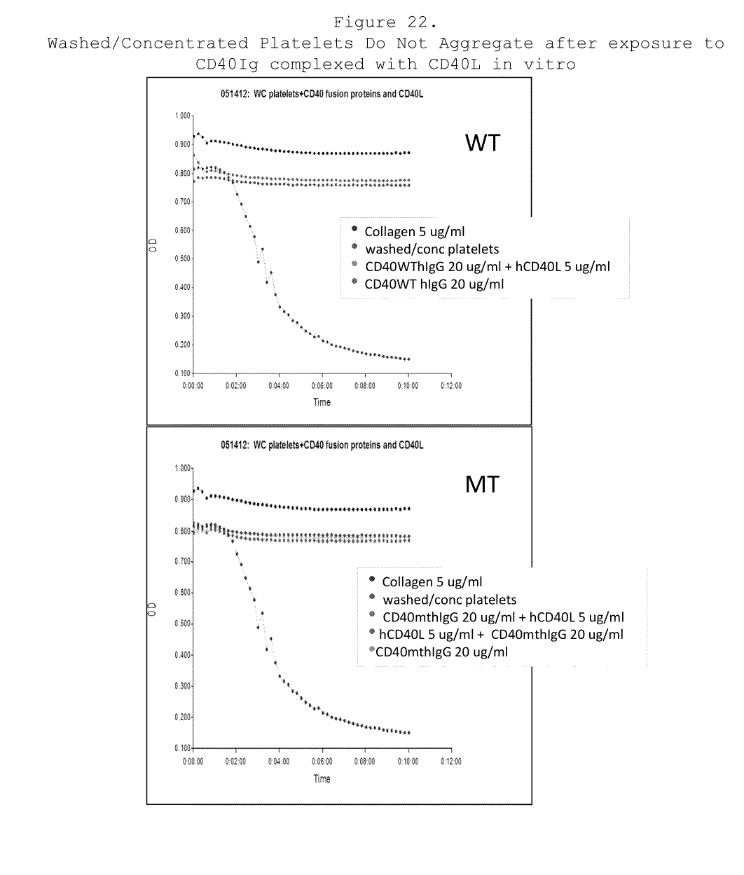

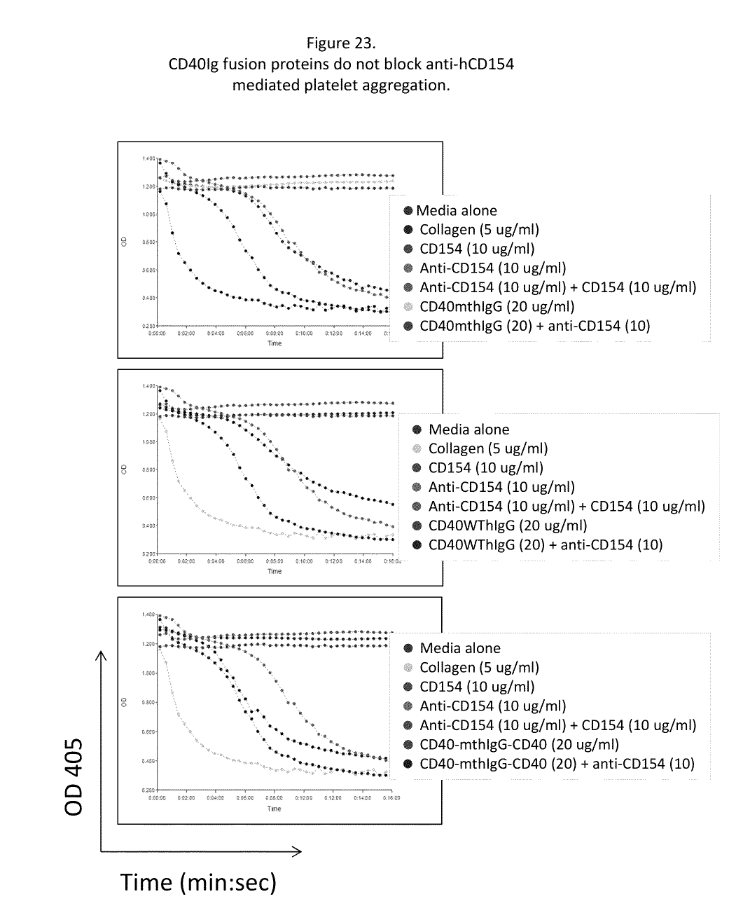

Because the antibody against human CD154 mediated platelet aggregation alone and this aggregation effect was augmented by formation of immune complexes between the antibody and soluble CD40L, similar assays were performed examining platelet aggregation mediated by immune complexes formed between soluble CD40L and the CD40ECIg fusion proteins. FIG. 22 shows two aggregation traces comparing the effects of the CD40-4s EC fusion proteins with either a wild type or mutant Fc domain. Surprisingly, neither fusion protein mediated platelet aggregation alone or after preformed immune complexes were added to individual wells of the assay. FIG. 23 shows a platelet aggregation assay where the platelets were pre-incubated for 30 minutes with CD40 fusion proteins prior to addition of other stimuli, and aggregation monitored as usual during a 45 minute kinetic run. The results are shown as OD traces as a function of time for several individual wells as indicated in the figure. The anti-CD 154 antibody and [antibody-CD154] immune complexes mediated platelet aggregation, while the CD40 fusion proteins alone did not. In addition, the fusion proteins were unable to block the aggregation mediated by CD40L (CD154) specific antibodies, indicating that the CD40Ig fusion proteins do not bind the same epitope on CD154 as the antibody used for the assay. Interestingly, unlike the CD40WTIg and CD40mtIg fusion proteins, the CD40IgCD40 fusion protein showed more rapid aggregation in the presence of the CD154 antibody, so that the aggregation profile was most similar to that of the [CD54 antibody-ligand] immune complexes than to the antibody alone.

### Example 18

### Immobilized Anti-CD40 Antibody Binds to CD40-Ig-LDLR Fusion Protein and then Captures Human PCSK9 Protein from Solution Through the Molecular Bridge

An antigen binding ELISA was performed in order to assess whether LDLR-CD40 fusion proteins are capable of simultaneously binding to both PCSK9 and to an anti-CD40 antibody. Briefly, ELISA plates were coated with 2-5 μg/ml capture antibody (either 2 ug/ml goat anti-human IgG (Jackson Immunoresearch, West Grove, Pa.) or 5 ug/ml mouse anti-human CD40 (Biolegend, San Diego, Calif.) in 50 microliters) and incubated overnight at 4° C. Plates were washed with PBS/0.05% Tween 20, blocked in PBS/2% BSA for 2 hours at room temperature, washed with PBS/0.05% Tween-20, and incubated with serial dilutions of COS7 culture supernatants from LDLR-CD40 construct(s), for at least 2 hours at room temperature. Duplicate plates were washed three times in PBS/0.05% Tween-20, then incubated with (a) 1:7500 goat anti-human IgG detection antibody (Catalog #109-036-003, Jackson Immunoresearch, West Grove, Pa.) conjugated with horseradish peroxidase or (b) with 5 μg/ml PCSK9-his 6 antigen (Catalog # PC9-H5223, AcroBiosystems, Bethesda, Md.) in D-PBS+Ca⁺⁺ and Mg⁺⁺ (Hyclone, ThermoFisher Scientific) and incubated 2 hours at room temperature or overnight at 4° C. Plates from step (b) were washed three times and incubated with HRP-anti-His tag antibody (Life Technologies, Grand Island, N.Y.) for 1.5 hours at room temperature.

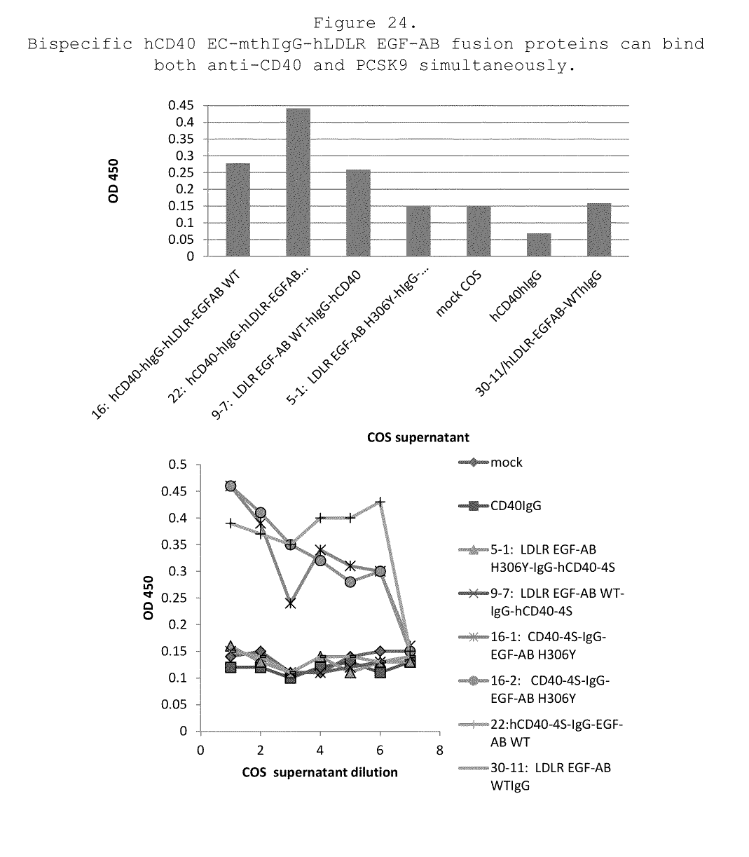

Plates from either step (a) or step (b) were washed four times in PBS/0.05% Tween-20, and incubated with 80 microliters of TMB substrate. Reactions were stopped by addition of 80 μl 1N HCl, and samples read at 450 nm on a SYNERGY® 2 Biotek plate reader (Biotek Instruments, Winooski, Vt.). Raw data was analyzed with Gen5 v2 software. The IgG sandwich ELISA estimated the amount of fusion protein present in the cultures relative to a concentration standard (CD40IgG purified protein) and a transfection standard (30-11, the LDLR EGF-AB-IgG transfected COS supernatant), indicating that there is very little fusion protein present in the LDLR EGF-AB-Ig-CD40 COS supernatants (5-1 and 9-7), while there is a much higher level of fusion protein present in the CD40-Ig-EGF AB (16-1, 16-2, and 22). The CD40-PCSK9 antigen binding ELISA demonstrated CD40 and EGF-AB simultaneous binding by capturing the fusion proteins through the CD40 domain and detecting captured fusion protein through PCSK9-his6 binding. The results from the CD40-PCSK9 antigen binding ELISA are shown in FIG. 24. The assay was performed twice on COS supernatants from repeated transfections with similar results.

### Example 19

### The Human LDLR EGF-AB Domain or the CD40 Extracellular Domain can be Mutated at Several Locations to Create a Higher Affinity PCSK9 or CD40L Binding Soluble -Ig Fusion Protein

Mutagenesis of proteins to increase binding affinity is a technique known in the art. Crystal structures of ligands and their cognate receptors have helped to identify critical amino acid residues and noncovalent interactions that contribute to the binding between fusion proteins. Techniques to isolate higher affinity fusion proteins are also well defined. Identifying protein variants that have increased binding affinity to their cognate ligand is a well-established technology in the art. Both targeted and random mutagenesis techniques have been successfully used to generate novel mutated protein variants, and analysis of their binding properties has identified novel variants with altered binding properties for one or more of the cognate ligands. CTLA4Ig-CD80/CD86 (Peach et al., 1994; Larsen et al., 2005) and human IgG1 (Idusogie E E et al., 2000 and 2001; Lazar et al., 2001; Shields et al., 2006) sequence variants have been generated and analysis of their binding properties have shown that some of these have increased affinity for one ligand and decreased affinity for other(s).

Fusion proteins with amino acid changes in the LDLR EGF-AB domain or in the CD40 EC domain may have altered or improved binding affinity for the PCSK9 or CD40L antigens. Identification of these increased binding affinity fusion proteins in the case of CD40 binding to CD154 or EGF AB to PCSK9 will be greatly aided by the crystal structures—reported in the following reference for CD40-CD154: An H J, Kim Y J, Song D H, Park B S, Kim H M, Lee J D, Paik, S G, Lee J O, and Lee H., Crystallographic and Mutational Analysis of the CD40-CD154 Complex and its Implication for Receptor Activation. J Biol Chem 286 (13): 11226-11235, (2011). In addition, sequence and domain alignments between homologous family members have been used to generate molecular models many proteins, including the CD28-CTLA4 family, the CD80/B7 family, and the TNFR families of proteins (Bajorath J, Marken J S, Chalupny J, Spoon T L, Siadak A W, Gordon M, Neelle R J, Hollenbaugh D, Aruffo A. Analysis of gp39/CD40 Interactions Using Molecular Models and Site Directed Mutagenesis. Biochemistry 34: 9884-9892, (1995).

The crystal structures identify important contact residues that play a critical role in receptor-ligand interactions and may be potential candidate residues for improving binding properties. Thus the structural analysis will aid in identification of directed changes. Nevertheless, approaches that target each amino acid individually in a sequence to test exhaustive amino acid substitutions is within the art. Several studies have reported mutagenesis of CD40 and CD154 amino acids and explored the alterations in binding properties of many of these fusion proteins. The majority of the mutants reduced or eliminated binding to the ligand as reported in the different studies. However, a few positions were found to tolerate significant charge, polarity, or size changes without significant effects on binding to the cognate ligand.

Certain CD40 mutant protein may be capable of binding CD40 ligand with greater affinity than CD40 wild type protein. Preferred mutations in CD40 are at residues 74, 76, 79, 81, 84, 85, 86, 93, 110, and 112-17, or within 10 amino acids of identified contact residues. Some preferred embodiments will be generated using the oligonucleotides listed in the sequence listing, SEQ ID NOs 85-132 and the CD40-4S extracellular domain as template in PCR amplification reactions. Introducing multiple mutations in the primary sequence where the predicted secondary and tertiary structures indicate the presence of multiple charge interactions or salt bridges with the ligand could be capable of introducing increased affinity. Complementary changes of two or more residues predicted to be in close proximity in three-dimensional space can be made at appropriate locations in the primary sequence, and may result in a higher affinity receptor-ligand interaction. An example of such a multiple mutant, higher affinity fusion protein is the L104E A29Y double mutant in CTLA4 (belatacept) that increases binding affinity of the CTLA4 fusion protein for its ligands CD80 (2×) and CD86 (4×). (Larsen C P, Pearson T C, Adams A B, Tso P, Shirasugi N, Strobert E, Anderson D, Cowan S, Price K, Naemura J, Emswiler J, Greene J, Turk L A, Bajorath J, Townsend R, Hagerty D, Linsley P S, and Peach R J. Rational Development of LEA29Y (belatacept), a High-Affinity Variant of CTLA4-Ig with Potent Immunosuppressive Properties. Amer J Transplantation 5: 443-453 (2005).)

Initial constructs described herein for expressing the CD40 extracellular domain as an -Ig fusion protein truncate the extracellular domain of human CD40 at several sites in the region between domains 3 and 4. In addition to adjusting the location of the domain 3-4 truncation, the CD40 extracellular domain can be mutated at several residues identified from crystal structure analysis as being important for functional interactions with CD40L amino acids. Preferred particular single or multiple mutants would include mutations or combinations of mutations at positions K46, D69, E74, H76, Q79, K81, D84, P85, N86, Q93, H110, T112, E114, and E117. Oligonucleotides used to introduce targeted mutations in the CD40 sequence are listed in SEQ ID NOs 85-132.

Mutating position K46 to K46T or K46H, maintains a polar or basic property at this position, since this amino acid in CD40 is predicted from the crystal structure to interact with residues Y146 and E142 of CD40L. Position E64 would be changed to E64S or E64Y, changing the acidic residue to a polar residue with an available hydroxyl group. Because CD40 E64 is predicted to interact with residues E129, K143, and K144 of CD40L, it is possible that the less acidic residue might be tolerated at this position. Position E66 and E74 would be changed to D or T residues to maintain an acidic or polar group for interaction with residues K143 or R200 and R203 of CD40L, respectively. Position D69 would be substituted with glutamine or asparagine to create a more conservative change than the alanine scanning mutation already reported. Amino acid H76 would be changed to glutamine (Q) to maintain a relatively large size and a polar interaction with amino acids G144, Y145, R203, and E230. Q79K or Q79Y would be preferred mutations at position Q79 to facilitate a hydrophilic contact with T251 of CD40L. Mutation of CD40 K81 to alanine was reported to slightly increase the binding of CD40 to CD40L (Bajorath J, Marken J S, Chalupny J, Spoon T L, Siadak A W, Gordon M, Neelle R J, Hollenbaugh D, Aruffo A. Analysis of gp39/CD40 Interactions Using Molecular Models and Site Directed Mutagenesis. Biochemistry 34: 9884-9892, (1995).)

Mutating K81H, K81S, or K81T will assess whether a more polar or basic residue at this position would also improve the binding in this region. Because this amino acid falls within a stretch of fairly basic, bulky or polar amino acids flanked by cysteines, these more conservative sequence changes may improve binding in this region while improving flexibility due to the incorporation of smaller amino acids. While a mutation at position D84 (D84E) was already reported by Singh et at to result in suboptimal binding to CD40L, the incorporation of this mutation with a second compensatory mutation at position E117 (E117D or E117Q) or possibly E114R or E114Q or N might result in improved charge interactions of these two amino acids with R207 in CD40L. E117 also engages in a hydrophilic contact with CD40L Q220 and D84 engages in an additional hydrophilic contact with S248 of CD40L and a charge interaction with CD40L H249, so there are multiple molecular contacts at play with both of these contact positions in CD40. Position P85 engages in a hydrophobic interaction with CD40L residues A130 and V247; therefore any potential mutations at this position should maintain the relatively bulky, uncharged nature of this position (i.e., mutating position P85 to tyrosine or tryptophan, with tryptophan being the preferred mutation). Although position N86 in CD40 has been identified as being critical for CD40L binding, it is possible that substitutions at this position that maintain the polarity might be tolerated, including N86Q or N86T. Although position T112 has not been identified as critical for binding, its proximity to the acidic region including E114-E117 suggests that it may play a role in stabilizing part of the binding interface (i.e., mutating this residue as well, to T112S or T112Y). A similar approach may be taken to target other single amino acid residues in CD40 or whole segments which either help stabilize the binding interface or form the binding interface with CD40L. These regions or residues may be selected for mutagenesis, expression, and screening for binding properties.

A similar targeted mutagenesis approach can be taken for the LDLR EGF AB domain using the PCSK9-LDLR EGFAB domain crystal structures as a guide for critical binding interactions. The structural analysis of the PCSK9-LDLR EGFAB domain interactions are described in the following references: Saha S, Boyd J, Werner J M, Knott V, Handford P A, Campbell A D, and Downing A K. Solution Structure of the LDL Receptor EGF-AB Pair: A Paradigm for the Assembly of Tandem Calcium Binding EGF Domains. Structure 9: 451-456 (2001). Kwon H J, McNutt M C, Horton J D, and Deisenhofer J. Molecular Basis for LDL receptor recognition by PCSK9. PNAS 105(6): 1820-1825 (2008). Bottomley M J, Cirillo A, Orsatti L, Ruggeri L, Fisher T S, Santoro J C, Cummings R T, Cubbon R M, Lo Surdo P, Calzetta A, Noto A, Baysarowich J, Mattu M, Talamo F, De Francesco R, Sparrow C P, Sitlani A, and Carfi A. Structural and Biochemical Characterization of the Wild Type PCSK9-EGF (AB) Complex and Natural Familial Hypercholesterolemia Mutants. J Biol Chem 284(2): 1313-1323 (2009).

Alternative embodiments of PCSK9 inhibitors or CD40 costimulation pathway inhibitors can be envisioned which are generated through scanning or targeted mutagenesis approaches and used as single mutants or in multiple combinations to construct higher affinity CD40L or PCSK9 binding fusion proteins. Methods for generation of random mutations or targeted mutations, molecular display, and affinity maturation of antigens have been described and are well known in the art.

### Example 20

### Substitution of Amino Acids at Selected CD40 Residues Improve CD40-Ig Expression Level and Binding to CD40L

A panel of CD40 mutants was generated by PCR mutagenesis methods that are well known in the art, and substitution mutants screened for expression and binding to CD40 ligand (CD40L). Briefly, the codon encoding the desired amino acid substitution in CD40 was included in overlapping, complementary oligonucleotides. These oligonucleotides are itemized in SEQ ID NOs: 87-132. The overlapping, complementary oligonucleotides were used to amplify sub-fragments of CD40 in combination with the amino or carboxy terminal oligonucleotide for CD40 attached to the human VK3 leader peptide (SEQ ID NOs: 85 and 86), so that the sense substituted oligonucleotide and the antisense carboxy oligonucleotide (SEQ ID NO: 86) were included in the same amplification reaction, while the antisense substituted oligonucleotide was combined with the amino terminal oligonucleotide (SEQ ID NO: 85) in the same amplification reaction. For both PCR reactions, the wild type CD40-Ig was included as template DNA. The amplified CD40 subfragments were then combined in overlap extension PCR reactions for two cycles, followed by addition of the amino and carboxy terminal primers (SEQ. ID NOS. 85-86) to amplify the altered full length CD40 cassette. PCR reactions were performed using a C1000 thermal cycler (BioRad, Hercules Calif.). Reactions included an initial denaturation step at 95° C. for 2 minutes, followed by 34 cycles with a 94° C., 30 sec denaturation, 50° C., 30 sec annealing, and 72° C., 1 minute extension step, followed by a final 4 minute extension at 72° C.

PCR fragments from the secondary reactions were purified using QIAQUICK® kits (QIAGEN, Valencia, Calif.), cloned into the pCR2.1 vector by TOPO cloning (Life Technologies, Grand Island, N.Y.), and plasmids prepared according to manufacturer's instructions for the QIAGEN miniprep kits (QIAGEN, Valencia, Calif.). Plasmid DNA was prepared from individual clones, and each sequence substitution(s) verified by DNA sequencing using ABI Dye Terminator v3.1 ready reaction sequencing mix according to manufacturer's instructions. For several of the multiple amino acid substitution mutants, the single mutant at one of the positions was used as template DNA for overlap extension PCR with oligonucleotides encoding the mutations at the second position.

The CD40-Ig fusion genes with correct amino acid substitutions were inserted into the mammalian expression vector pDG, and DNA from positive clones was amplified using QIAGEN plasmid preparation kits (QIAGEN, Valencia, Calif.). Mini-plasmid preparations (2.5 ug DNA for 60 mm plates, 1.5 ug for 6 well plates) were used for COST transfections using the QIAGEN POLYFECT® reagent and following the manufacturer's instructions. Culture supernatants were harvested 48-72 hours after transfection, and filtered through 0.22 um syringe filter units.

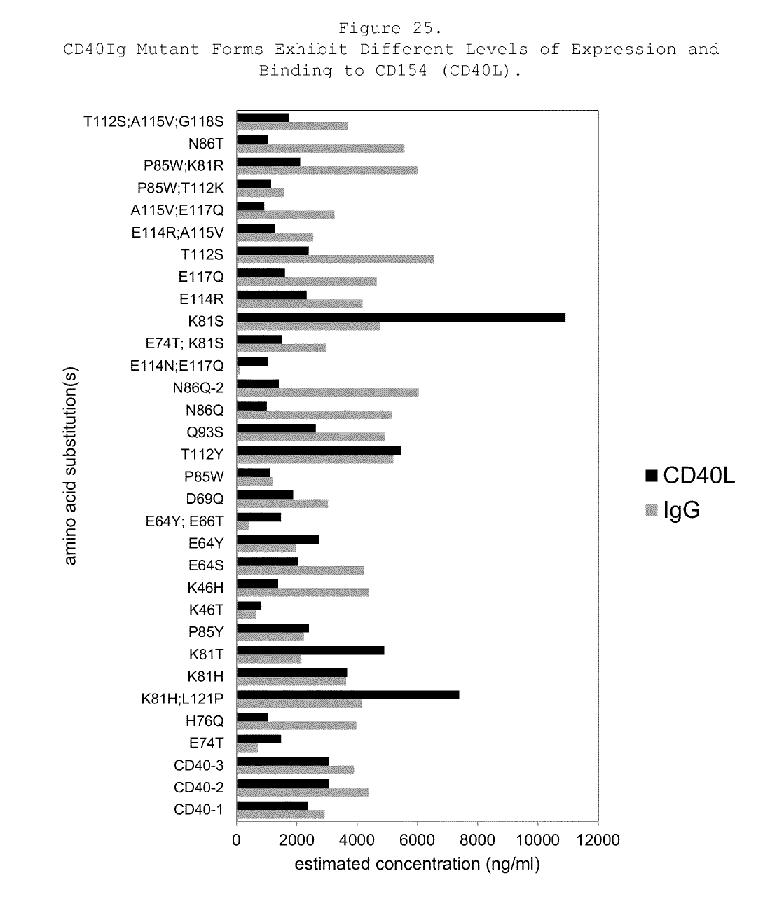

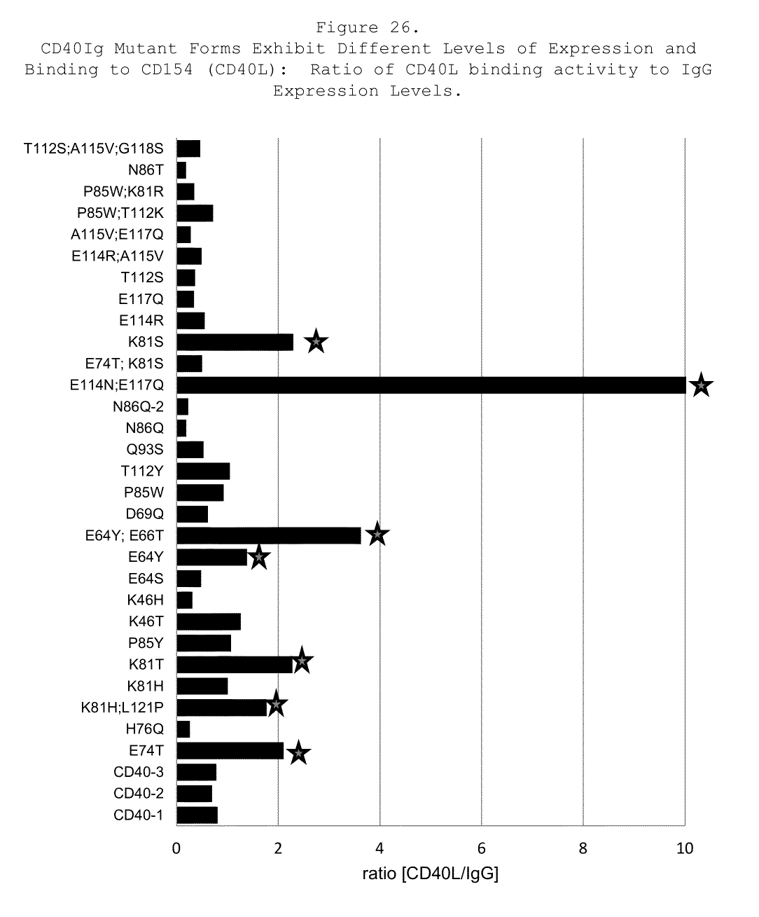

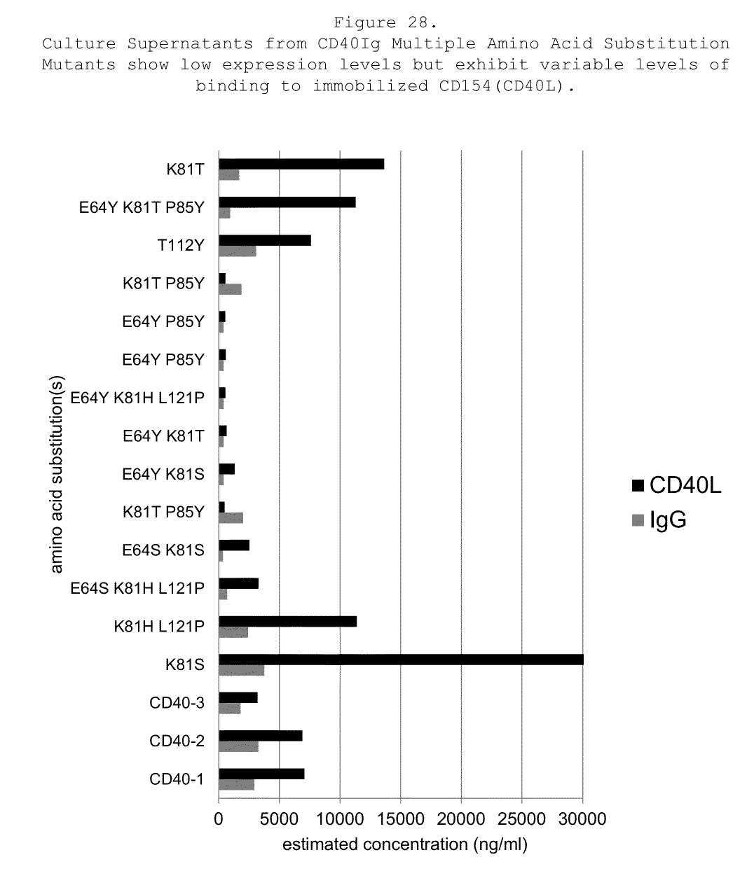

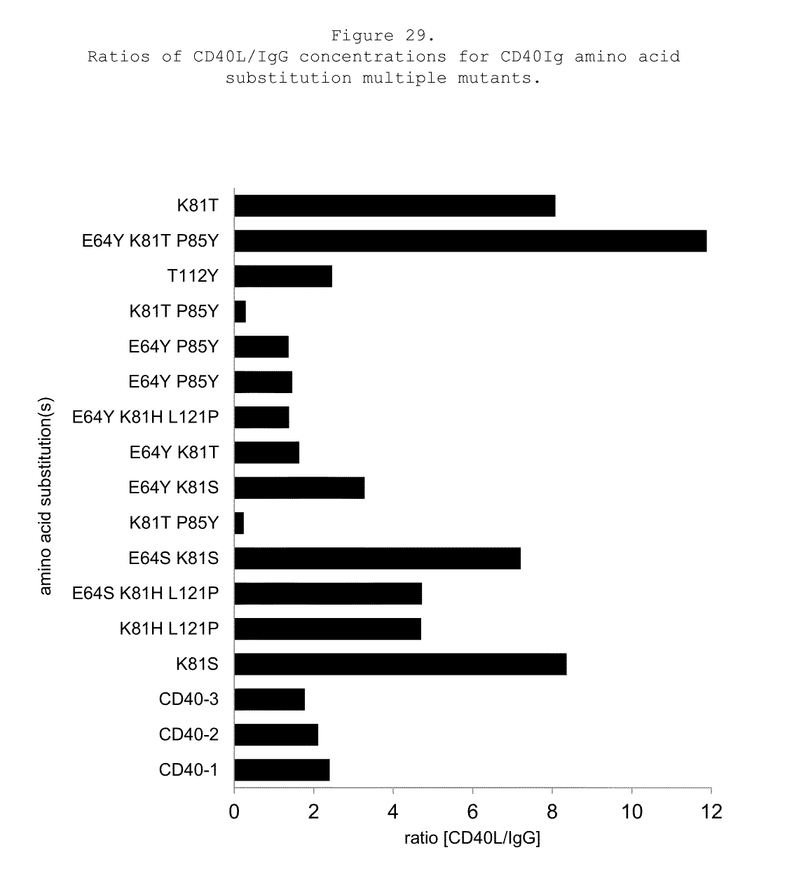

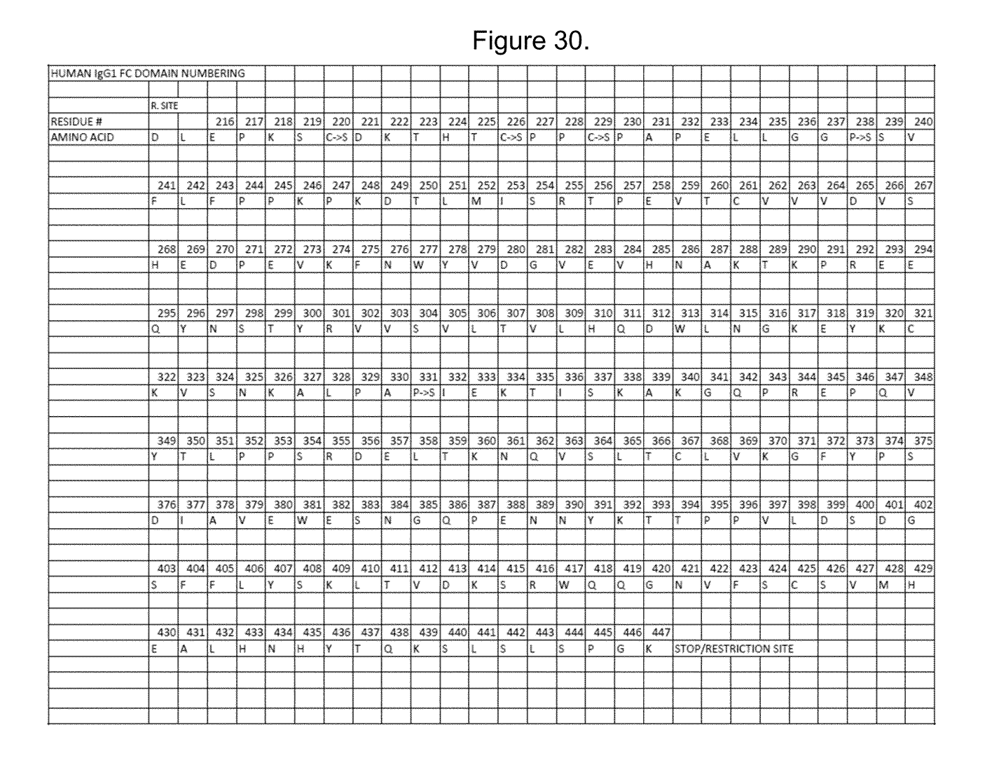

Culture supernatants from COS transfections were assayed using parallel IgG and CD40L antigen binding sandwich ELISAs. NUNC MAXISORP® plates were coated overnight at 4° C. with 2 ug/ml F(ab′)2 goat anti-human IgG (Fc specific) (Catalog #109-006-008, Jackson Immunoresearch, West Grove, Pa.) in PBS for IgG ELISAS, or with 2.5 ug/ml recombinant human CD40L in PBS (Catalog #310-02, Peprotech, Rocky Hill, N.J.) for CD40L antigen binding ELISAs. Coated plates were washed twice in wash buffer: (PBS/0.05% Tween-20/0.01% Kathon), and blocked overnight at 4° C. in 200 ul/well PBS/2.5% BSA. Blocked plates were washed three times in wash buffer, and serial dilutions of each culture supernatant in (D-PBS, 0.2% BSA) added to successive wells in each plate: (20×, 40×, 80×, and 160× of each supernatant). Supernatant dilutions were incubated overnight at 4° C. Plates were washed three times in wash buffer, then incubated with HRP (horseradish peroxidase) conjugated goat anti-human IgG (Fc specific) (Catalog #109-036-008, Jackson Immunoresearch, West Grove, Pa.) at a 1:7,500 dilution in PBS for 1-2 hours at room temperature. Plates were washed 4-5 times in wash buffer, then 80 ul/well TMB Sureblue peroxidase substrate was added (Catalog #52-00-02, KPL Laboratories, Gaithersburg, Md.). Color was allowed to develop for 5-15 minutes, and reactions stopped by addition of 80 ul/well 1 N HCL. Plates were read using a Biotek SYNERGY® 2 (Biotek Instruments, Winooski, Vt.) plate reader at 450 nM, and data analyzed with Gen5 v2 software. The IgG sandwich ELISA estimated the amount of fusion protein present in the supernatants, while the CD40L antigen binding ELISA estimated the relative binding activity of each fusion protein cultures relative to a concentration standard (CD40WT-IgG purified protein) and a transfection standard (wild type CD40Ig (CD40WTIgG #1, 2, 3, three different transfected replicate supernatants). The CD40L-anti-IgG antigen binding ELISA demonstrated CD40L binding by capturing the fusion proteins through the CD40L-CD40 domain interactions and detecting captured fusion protein through anti-IgG binding. The results from the two different ELISAs are summarized in FIGS. 25 and 28. The assays were performed three times on COS supernatants from repeated transfections with similar results. The level of fusion protein detected in the IgG sandwich ELISA was also used to normalize the amount of CD40L binding detected for each clone, so that a ratio between the expression level and the level of CD40 ligand binding was generated for each sample. The ratio of binding to IgG and CD40L binding level was tabulated as shown in FIGS. 26 and 29.

In order to assay the relative CD40L binding levels, the ratio of CD40L binding compared to the level of protein expression was tabulated. Although most amino acid substitutions resulted in ratios equal to or less than the CD40 wild type controls, several substitution mutants were identified as showing a [CD40L]/[IgG] ratio significantly above the CD40 wild type controls. For position 64, substitution of E (glutamic acid) with serine (S) or tyrosine (Y) resulted in increased binding to CD40L on Jurkat cells, although the increase was more significant with serine at this position. However, when the relative expression level of each mutant was compared, the tyrosine substitution at position 64 gave a higher level of CD40L binding relative to the amount of protein. The E64Y E66T double substitution mutant showed significantly increased CD40L binding activity per IgG expressed.

For residue K81, substitution with serine (S), threonine (T), or histidine (H), all resulted in a marked increase in CD40L binding compared to wild type. The K81S and K81T mutants both showed high expression level of IgG and high CD40L binding activity relative to wild type CD40Ig.

For residue T112, substitutions were made with either serine (S) or tyrosine (Y). The T112Y mutant resulted in an increase in CD40L binding relative to the amount of IgG expressed, although the increase was marginal or undetectable for the T112S mutant. In addition to single amino acid substitution mutants, several double or triple mutant versions of CD40IgG were generated. The E114N E117Q double substitution mutant did not express well; however, the protein bound very well to immobilized rCD40L in the antigen binding ELISA (FIG. 29).

In addition, multiple substitution CD40 mutants at positions E64 and K81 were generated, including E64S K81S, E64S K81T, E64S K81H L121P, and E64Y K81T and E64Y K81H L121P. The E64S K81S and E64S K81H L121P mutants showed a higher level of CD40L binding relative to the amount of fusion protein expressed. The triple mutant E64Y K81T P85Y also showed a high level of CD40L binding in the culture supernatants relative to its expression level in COS 7 transfections.

It is demonstrated here that targeted mutagenesis strategies may be employed to generate CD40 fusion proteins with improved binding affinity for CD40L. Several positions were identified as important in forming ligand-receptor contacts or adjacent residues from the crystal structures and sequence and domain alignments between homologous family members. Substitutions at most of the contact residues resulted in decreased expression and CD40L binding. Some mutants expressed at high levels, but showed little or no CD40L binding activity. However, we identified a few residues that can be substituted with alternative amino acids in order to generate fusion proteins with improved avidity for CD154 (CD40L). In particular, E64 and L121, E66, K81, P85, T112, E114 and E117, when substituted with particular polar or conservative amino acid alternatives, exhibit increased levels of CD40L binding.

Several of the multiple amino acid substitution mutants also showed increased CD40L binding activity relative to their expression level, indicating their potential utility as CD40-CD40L pathway inhibitors. The E64Y E66T; E114N E117Q; E64Y K81T P85Y; E64S K81S; E64S K81H L121P; K81H L121P multiple substitution mutants of CD40IgG showed a surprising and significant increase in the ratio of CD40L binding avidity relative to IgG expression level.

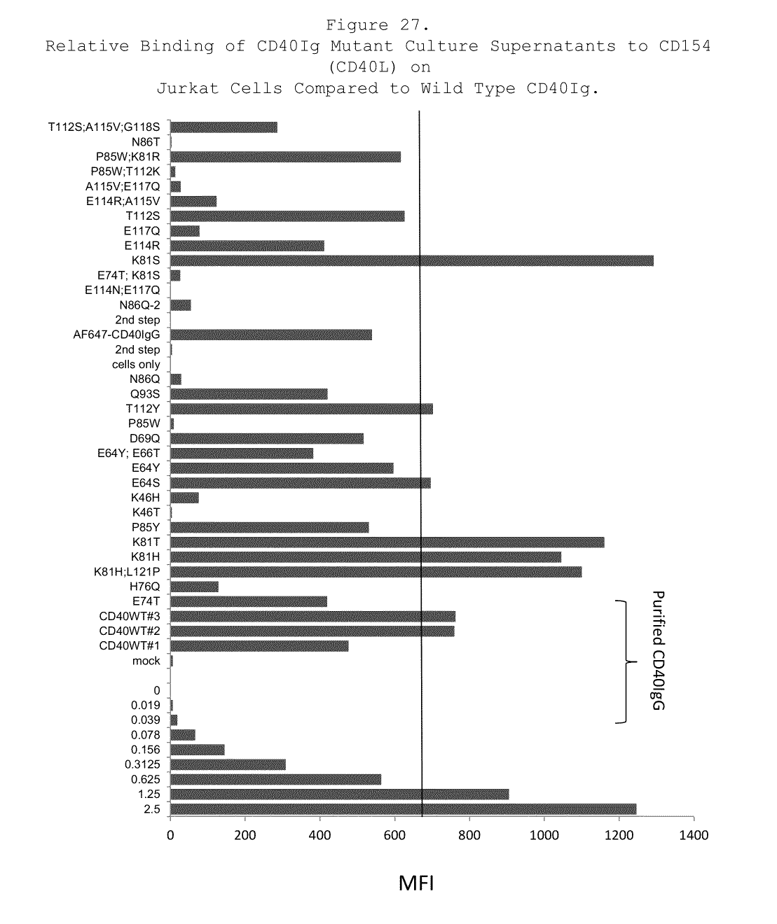

In addition to the ELISA measures of CD40L binding, several of the supernatants from COS transfections were also assayed for binding to CD154 (CD40L) expressed on the surface of Jurkat cells. Jurkat cells in logarithmic growth were harvested by centrifugation at 1100 rpm, and the cells washed and re-suspended in D-PBS/3% FBS. Cells (approximately 1×10⁶/well) were aliquoted to 96 well U-bottom microtiter plates and centrifuged at 1400 rpm, 4 C, for 10 minutes. Cells were re-suspended in COS supernatants or dilutions of purified CD40WTIgG fusion proteins (100 ul/well), and incubated for one hour on ice. Plates were centrifuged, and cells washed twice in D-PBS/3% FBS, prior to staining in 1:800 Alexafluor 647 goat anti-human IgG (Life Technologies, Gaithersburg, Md.) in PBS/3% FBS. Cells were incubated in second step reagent for 45-60 minutes on ice, then centrifuged, and washed two times in PBS/3% FBS, 200 ul/well. Cells were re-suspended in 200 ul PBS/3% FBS, and transferred to tubes for analysis by flow cytometry. Stained cells were analyzed using a FACS-Canto flow cytometer (Becton-Dickinson, San Jose, Calif.), and data analysis performed using FlowJo software (Treestar, Ashland, Oreg.). The mean fluorescence intensity for each sample was then plotted and compared to that for purified CD40WTIgG. Data are shown in FIG. 27. The sensitivity of the cell surface binding assay was insufficient to accurately measure binding for several of the lower expressing fusion proteins; however, the ELISA assay with immobilized CD40L was more sensitive by more than an order of magnitude, facilitating screening of these fusion proteins and assessment of their relative binding avidities.

### Example 21

### Extended LDLR-Domain Fusions

Several studies have indicated that the LDLR EGFP modules interact extensively through side chains that form conserved packing and hydrogen bonding interactions in the interior and between the propeller blades of the β-propeller motif and also over hydrophobic interfaces between domains that facilitate their function (Jeon et al, 2001). In addition to the examples described previously, new LDLR forms may be envisioned that include not only the LDLR EGF A-B domains, but the adjacent beta-propeller domain and even the EGF-C domain of LDLR. These sequences have been described by Jeon et al, 2001; Beglova et al., 2004; Bottomley et al., 2009; Yamamoto et al., 2011; Lo Surdo et al., 2012. Crystal structures indicate that the β-propeller-YTWD containing domains may add stability to the LDLR EGF-AB domain, assist in binding to PCSK9, and release of ligand. Similarly, the LDLR EGF-C domain protects a hydrophobic binding interface on the LDLR that may improve expression, solubility, and binding to the native ligand (Jeon et al., 2001). These alternative truncated LDLR forms may therefore encode LDLR EGF-AB-β-propeller as listed in SEQ ID NOs: 143, 144, 145, or 146. Fusions to the CD40 extracellular domain are also envisioned, as listed in SEQ ID NO. 147 or 148 for 3′ fusions. Alternative configurations using these modular cassettes may also be envisioned.

Fusion proteins which encode the LDLR EGF-AB domain-β-propeller-and LDLR EGF-C domain are also envisioned and several preferred embodiments are listed in SEQ ID NOS 137, 138, 139, 140, 141, and 142, 167, 168, 169, 170, 171, 172, 173, and 174. Similarly, these fusion proteins may be expressed as multispecific fusion proteins with CD40-Ig forms, as listed in SEQ ID NOs 149 and 150. Alternative configurations or arrangements can also be envisioned using these cassettes.

### Example 22

### Construction and Expression of Anti-PCSK9-CD40 Multispecific Fusion Proteins

Higher affinity PCSK9 inhibitors are envisioned where a single chain variable region (scFv) for a PCSK9 binding antibody is fused to the CD40 extracellular domain. The sequence for one high affinity anti-PCSK9 antibody has been reported as a protein sequence from crystal structures of the antibody bound to soluble PCSK9 (Liang et al., 2012). This sequence is listed in the protein databases under accession number 3SQO_H for the heavy chain Fab sequence of antibody J16, and under 3SQO_L for the light chain Fab sequence of antibody J16. If the peptide sequence for this antibody is back translated, an scFv might be assembled by those of ordinary skill in the art using overlapping oligonucleotides or gene synthesis approaches. Some examples of preferred embodiments envisioned using this approach are listed in SEQ ID NOs: 151, 152, 153, and 154. Similarly, other groups (Chan et al., 2009; Ni et al., 2010; Ni et al., 2011; Chapparo-Riggers et al., 2012) have reported isolation, sequences, or crystal structures for anti-PCSK9 antibodies that might be assembled into a multi-functional receptor-scFv fusion protein targeting both CD40-CD40L and LDLR-PCSK9 interactions.

### Example 23

### Use of Teachable Examples from the Structure of LDLR Domains in Designing Novel Linkers for Fusion Proteins

The structure(s) reported for the LDLR by several of the studies cited indicate that the LDLR has linker like domains that might be useful in the construction and expression of other novel fusion proteins. The fusion protein includes both rigid linkers and more flexible linker like domains, so that depending on the functional properties desired for newly envisioned fusion proteins, different linkers might be useful to attach between the different domains of these novel fusion proteins. The LA modules of the ligand-binding domain of LDLR are connected by short four or five amino acid linkers (Kurniawan, et al., 2000; Beglova, et al., 2001; Beglova et al., 2004), except for a 12-residue linker located between the L4-L5 repeats. Solution NMR studies indicate that the linkers are all flexible, and allow adjacent domains to move freely with respect to each other. However, all the modules except the segment linking LDLR EGF-B to the β-propeller in the EGFP segment of the LDLR (EGF A-B-beta-propeller-C) are connected via rigid linker like sequences (Beglova, et al., 2004). This segment is longer, composed of both hydrophobic and charged residues, and shows varying degrees of X-ray structure depending on pH.

Preferred examples of more rigid linkers would be those from the EGFP segment of the LDLR. Short linkers are found in proteins with fixed orientation between adjacent domains and also in those with significant mobility between domains (Beglova et al., 2001). The shorter, more flexible linkers from the tandem repeat units of the ligand binding domain of the LDLR (Kurniawan, et al., 2000) might be useful as another preferred example should a short, flexible type of linker be desirable. Such short sequences might include VGDR (SEQ ID NO 155), LSVT (SEQ ID NO 156), PPKT (SEQ ID NO 157), PVLT (SEQ ID No 158), AVAT (SEQ ID NO 159), VNVTL (SEQ ID NO 160). Short, but possibly more constrained linkers might include PPQ (SEQ ID No 161), HQHPPG (SEQ ID NO 162). A longer linker similar to that between LB4 and LB5 of the ligand binding domain repeats might include the residues (SEQ ID NO 163) RGLYVFQGDSSP.

If a more flexible linker like domain is desirable, the segment between the LDLR EGF-AB domains and the β-propeller domain which includes all or part of the sequence (K)AVGS(IA) (SEQ ID NO: 164) might also be useful as part of a linker domain sequence. More rigid, less flexible linkers might also include HNLTQP(RG) (SEQ ID NO: 165), and QGDSSP (SEQ ID NO: 166) between domains where a less flexible, but less ordered segment is desired. These examples from the LDLR show that fusion proteins with a modular organization of multiple functions often contain peptide segments that might be utilized for construction of novel linkers for new fusion protein. Some of the examples between the LDLR functional domains illustrate that depending on the functional properties of each domain, the linker domain might also be optimized so adjacent domains function properly.

It will also be understood by one of ordinary skill in the art that the hybrid fusion proteins and/or proteins of the invention may be altered such that they vary in sequence from the naturally occurring or native sequences from which they were derived, while retaining the desirable activity of the native sequences. For example, nucleotide or amino acid substitutions leading to conservative substitutions or changes at “non-essential” amino acid residues may be made. An isolated nucleic acid fusion protein encoding anon-natural variant of a hybrid nuclease fusion protein derived from an immunoglobulin (e.g., an Fc domain) can be created by introducing one or more nucleotide substitutions, additions or deletions into the nucleotide sequence of the immunoglobulin such that one or more amino acid substitutions, additions or deletions are introduced into the encoded protein. Mutations may be introduced by standard techniques, such as site-directed mutagenesis and PCR-mediated mutagenesis.

The peptide hybrid fusion proteins of the invention may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members. Alternatively, in another embodiment, mutations may be introduced randomly along all or part of a coding sequence, such as by saturation mutagenesis, and the resultant mutants can be incorporated into binding polypeptides of the invention and screened for their ability to bind to the desired target.

It is contemplated that the mutagenesis mutants and/or mutations presented herein may be achieved through methods and techniques understood in the art. Some of these methods include, but are not limited to: random mutagenesis, site-directed mutagenesis, combinatorial mutagenesis, insertional mutagenesis, directed and/or PCR mutagenesis, scanning mutagenesis, yeast display methods, and/or so forth. See, for example, the techniques described in Sambrook et al., 2001, Molecular Cloning A Laboratory Manual, 3rd Ed., Cold Spring Harbor Laboratory, Cold Spring Harbor, N.Y.; and Ausubel et al., eds., 1998, Current Protocols in Molecular Biology, John Wiley & Sons, NY, which are both incorporated by reference herein in their entireties.

Additional peptide hybrid fusion proteins may be generated through the techniques of gene-shuffling, motif-shuffling, exon-shuffling, and/or codon-shuffling (collectively referred to as “DNA shuffling”). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308 (each of these patents and publications are hereby incorporated by reference in its entirety).

Yeast display type methods can be used with the present disclosure and are described in Wittrup K D (2007). Yeast surface display for protein engineering and characterization. Curr Opin Struct Biol 17:467-473; Glycobiology vol. 18 no. 2 pp. 137-144, 2008, Fishing for lectins from diverse sequence libraries by yeast surface display—An exploratory study. Ryckaert et al.; and Anal Biochem. 2012 Jan. 15; 420(2):163-70. doi: 10.1016/j.ab.2011.09.019. Epub 2011 Sep. 22, and U.S. Pat. Nos. 8,216,574 and 8,192,737, all of which are incorporated by reference herein in their entirety.

While the invention has been particularly shown and described with reference to an aspect and various alternate aspects, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES

1. Dahlof B. Cardiovascular disease risk factors: epidemiology and risk assessment. Am J Cardiol. 2010; 105(1 Suppl):3A-9A.
2. Baigent C, Blackwell L, Emberson J, Holland L E, Reith C, Bhala N, et al. Efficacy and safety of more intensive lowering of LDL cholesterol: a meta-analysis of data from 170,000 participants in 26 randomised trials. Lancet. 2010; 376(9753):1670-81. PMCID: 2988224.
18. Zhang D W, Garuti R, Tang W J, Cohen J C, Hobbs H H. Structural requirements for PCSK9-mediated degradation of the low-density lipoprotein receptor. Proc Natl Acad Sci USA. 2008; 105(35):13045-50. PMCID: 2526098.
19. Surdo P L, Bottomley M J, Calzetta A, Settembre E C, Cirillo A, Pandit S, et al. Mechanistic implications for LDL receptor degradation from the PCSK9/LDLR structure at neutral pH. EMBO Rep. 2011; 12(12):1300-5. PMCID: 3245695.
20. Zhang D W, Lagace T A, Garuti R, Zhao Z, McDonald M, Horton J D, et al. Binding of proprotein convertase subtilisin/kexin type 9 to epidermal growth factor-like repeat A of low density lipoprotein receptor decreases receptor recycling and increases degradation. J Biol Chem. 2007; 282(25):18602-12.
21. Graham M J, Lemonidis K M, Whipple C P, Subramaniam A, Monia B P, Crooke S T, et al. Antisense inhibition of proprotein convertase subtilisin/kexin type 9 reduces serum LDL in hyperlipidemic mice. J Lipid Res. 2007; 48(4): 763-7.
22. Duff C J, Scott M J, Kirby I T, Hutchinson S E, Martin S L, Hooper N M. Antibody-mediated disruption of the interaction between PCSK9 and the low-density lipoprotein receptor. Biochem J. 2009; 419(3):577-84. PMCID: 2669217.
23. Stein E A, Mellis S, Yancopoulos G D, Stahl N, Logan D, Smith W B, et al. Effect of a monoclonal antibody to PCSK9 on LDL cholesterol. N Engl J Med. 2012; 366(12):1108-18.
24. Shan L, Pang L, Zhang R, Murgolo N J, Lan H, Hedrick J A. PCSK9 binds to multiple receptors and can be functionally inhibited by an EGF-A peptide. Biochem Biophys Res Commun. 2008; 375(1):69-73.
25. McNutt M C, Kwon H J, Chen C, Chen J R, Horton J D, Lagace T A. Antagonism of secreted PCSK9 increases low density lipoprotein receptor expression in HepG2 cells. J Biol Chem. 2009; 284(16):10561-70. PMCID: 2667743.
26. Malby S, Pickering R, Saha S, Smallridge R, Linse S, Downing A K. The first epidermal growth factor-like domain of the low-density lipoprotein receptor contains a noncanonical calcium binding site. Biochemistry. 2001; 40(8):2555-63.
27. Libby P, Ridker P M, Hansson G K. Inflammation in atherosclerosis: from pathophysiology to practice. J Am Coll Cardiol. 2009; 54(23):2129-38. PMCID: 2834169.
28. Ross R. Atherosclerosis is an inflammatory disease. Am Heart J. 1999; 138(5 Pt 2):S419-20.
35. Huo Y, Schober A, Forlow S B, Smith D F, Hyman M C, Jung S, et al. Circulating activated platelets exacerbate atherosclerosis in mice deficient in apolipoprotein E. Nat Med. 2003; 9(1):61-7.
36. Noelle R J, Roy M, Shepherd D M, Stamenkovic I, Ledbetter J A, Aruffo A. A 39-kDa protein on activated helper T cells binds CD40 and transduces the signal for cognate activation of B cells. Proc Natl Acad Sci USA. 1992; 89(14):6550-4. PMCID: 49539.
37. Ludewig B, Henn V, Schroder J M, Graf D, Kroczek R A. Induction, regulation, and function of soluble TRAP (CD40 ligand) during interaction of primary CD4+CD45RA+ T cells with dendritic cells. Eur J Immunol 1996; 26(12):3137-43.
38. Noelle R J, Ledbetter J A, Aruffo A. CD40 and its ligand, an essential ligand-receptor pair for thymus-dependent B-cell activation. Immunol Today. 1992; 13(11):431-3.
39. Marshall L S, Aruffo A, Ledbetter J A, Noelle R J. The molecular basis for T cell help in humoral immunity CD40 and its ligand, gp39. J Clin Immunol. 1993; 13(3):165-74.
40. Kleemann R, Zadelaar S, Kooistra T. Cytokines and atherosclerosis: a comprehensive review of studies in mice. Cardiovasc Res. 2008; 79(3):360-76. PMCID: 2492729.
41. Zernecke A, Shagdarsuren E, Weber C. Chemokines in atherosclerosis: an update. Arterioscler Thromb Vasc Biol. 2008; 28(11):1897-908.
42. Schonbeck U, Libby P. CD40 signaling and plaque instability. Circ Res. 2001; 89(12):1092-103.
43. Mach F, Schonbeck U, Sukhova G K, Atkinson E, Libby P. Reduction of atherosclerosis in mice by inhibition of CD40 signalling. Nature. 1998; 394(6689):200-3.
44. Lutgens E, Gorelik L, Daemen M J, de Muinck E D, Grewal I S, Koteliansky V E, et al. Requirement for CD154 in the progression of atherosclerosis. Nat Med. 1999; 5(11):1313-6.
45. Lutgens E, Cleutjens K B, Heeneman S, Koteliansky V E, Burkly L C, Daemen M J. Both early and delayed anti-CD40L antibody treatment induces a stable plaque phenotype. Proc Natl Acad Sci USA. 2000; 97(13):7464-9. PMCID: 16568.
46. Schonbeck U, Sukhova G K, Shimizu K, Mach F, Libby P Inhibition of CD40 signaling limits evolution of established atherosclerosis in mice. Proc Natl Acad Sci USA. 2000; 97(13):7458-63. PMCID: 16567.
47. Boumpas D T, Furie R, Manzi S, Illei G G, Wallace D J, Balow J E, et al. A short course of BG9588 (anti-CD40 ligand antibody) improves serologic activity and decreases hematuria in patients with proliferative lupus glomerulonephritis. Arthritis Rheum. 2003; 48(3):719-27.
48. Vincenti F. What's in the pipeline? New immunosuppressive drugs in transplantation. Am J Transplant. 2002; 2(10):898-903.
49. Langer F, Ingersoll S B, Amirkhosravi A, Meyer T, Siddiqui F A, Ahmad S, et al. The role of CD40 in CD40L- and antibody-mediated platelet activation. Thromb Haemost. 2005; 93(6):1137-46.
50. Robles-Carrillo L, Meyer T, Hatfield M, Desai H, Davila M, Langer F, et al. Anti-CD40L immune complexes potently activate platelets in vitro and cause thrombosis in FCGR2A transgenic mice. J Immunol. 2010; 185(3):1577-83.
An H J, Kim Y J, Song D H, Park B S, Kim H M, Lee J D, Paik, S G, Lee J O, and Lee H., Crystallographic and Mutational Analysis of the CD40-CD154 Complex and its Implication for Receptor Activation, (2011), J Biol Chem 286 (13): 11226-11235
Bajorath J, Marken J S, Chalupny J, Spoon T L, Siadak A W, Gordon M, Neelle R J, Hollenbaugh D, Aruffo A. Analysis of gp9/CD40 Interactions Using Molecular Models and Site Directed Mutagenesis. (1995) Biochemistry 34: 9884-9892.
Bajorath J, and Aruffo A. Construction and Analysis of a Detailed Three-Dimensional Model of the Ligand Binding Domain of the Human B Cell Receptor CD40. (1997) Proteins: Structure, Function, and Genetics 27: 59-70.
Bottomley M J, Cirillo A, Orsatti L, Ruggeri L, Fisher T S, Santoro J C, Cummings R T, Cubbon R M, Lo Surdo P, Calzetta A, Noto A, Baysarowich J, Mattu M, Talamo F, De Francesco R, Sparrow C P, Sitlani A, and Carfi A. Structural and Biochemical Characterization of the Wild Type PCSK9-EGF (AB) Complex and Natural Familial Hypercholesterolemia Mutants. J Biol Chem 284(2): 1313-1323 (2009).
Idusogie E E, Presta L G, Gazzano-Santoro H, Totpal K, Wong P Y, Ultsch M, Meng Y G, Mulkerrin M G. Mapping of the C1q binding site on rituxan, a chimeric antibody with a human IgG1 Fc. (2000) J Immunol. 164(8):4178-84.
Idusogie E E, Wong P Y, Presta L G, Gazzano-Santoro H, Totpal K, Ultsch M, Mulkerrin M G. Engineered antibodies with increased activity to recruit complement. (2001) J Immunol. 166(4):2571-5.
Kwon H J, McNutt M C, Horton J D, and Deisenhofer J. Molecular Basis for LDL receptor recognition by PCSK9. PNAS 105(6): 1820-1825 (2008).
Lazar G A, Dang W, Karki S, Vafa O, Peng J S, Hyun L, Chan C, Chung H S, Eivazi A, Yoder S C, Vielmetter J, Carmichael D F, Hayes R J, Dahiyat B I. Engineered antibody Fc variants with enhanced effector function. (2006) PNAS 103(11): 4005-4010.
Peach R J, Bajorath J, Brady W, Leytze G, Greene J, Naemura J, and Linsley P S. Complementarity Determining Region 1 (CDR1)- and CDR3-analogous Regions in CTLA-4 and CD28 Determine the Binding to B7-1. (1994) J Exp Med 180: 2049-2058.
Peach R J, Bajorath J, Naemura J, Leytze G, Greene J, Aruffo A, and Linsley P S. Both Extracellular Immunoglobin-like Domains of CD80 Contain Residues Critical for Binding T Cell Surface Receptors CTLA and CD28. (1995) J Biol Chem 270(36): 2118-21187.
Saha S, Boyd J, Werner J M, Knott V, Handford P A, Campbell A D, and Downing A K. Solution Structure of the LDL Receptor EGF-AB Pair: A Paradigm for the Assembly of Tandem Calcium Binding EGF Domains. Structure 9: 451-456 (2001).
Schwartz J C D, Zhang X, Fedorov A A, Nathenson S G, and Almo S C. Structural Basis for co-stimulation by the human CTLA-4/B7-2 complex. (2001) Nature 410: 604-608.
Shields R L, Namenuk A K, Hong K, Meng Y G, Rae J, Briggs J, Xie D, Lai J, Stadlen A, Li B, Fox J A, Presta L G. High resolution mapping of the binding site on human IgG1 for Fc gamma RI, Fc gamma RII, Fc gamma RIII, and FcRn and design of IgG1 variants with improved binding to the Fc gamma R. (2001) J Biol Chem. 276(9):6591-604.
Singh J, Garber E, Van Vlijmen H, Karpusas M, Hsu Y M, Zheng Z, Naismith J H and Thomas D. The role of polar interactions in the molecular recognition of CD40L with its receptor CD40. (1998) Protein Science 7: 1124-1135.
Stamper C C, Zhang Y, Tobin J F, Erbe D V, Ikemizu S, Davis S J, Stahl M L, Seehra J, Somers W S, and Mosyak L. Crystal Structure of the B7-1/CTLA-4 complex that inhibits human immune responses. (2001) Nature 410: 608-611.
Kurniawan N D, Atkins A R, Bieri S, Brown C J, Brereton I M, Kroon P A, and Smith S. NMR Structure of a concatemer of the first and second ligand-binding modules of the human low-density lipoprotein receptor. Protein Science 9: 1282-1293 (2000).
Beglova N, North C L, and Blacklow S C, Backbone Dynamics of a Module Pair from the ligand binding domain of the LDL Receptor. Biochemistry 40: 2808-22815 (2001).
Jeon H, Meng W, Takagi J, Eck M J, Springer T A, and Blacklow S C. Implications for familial hypercholesterolemia from the structure of the LDL receptor YWTD-EGF domain pair. Nature Structural Biology 8(6): 499-504 (2001).
Herz J Deconstructing the LDL receptor—a rhapsody in pieces. Nature Structural Biology 8(6): 476-478 (2001).
Beglova N, Jeon H, Fisher C, and Blacklow S C, Structural features of the low-density lipoprotein receptor facilitating ligand binding and release. Biochemical Society Transactions 32 (5): 721-723 (2004).
Zhang D W, Garuti R, Tang W J, Cohen J C, and Hobbs H H. Structural requirements for PCSK9-mediated degradation of the low-density lipoprotein receptor. PNAS 105(35): 13045-13050 (2008).
Bottomley M J, Cirillo A, Orsatti L, Ruggeri L, Fisher T S, Santoro J C, Cummings R T, Cubbon R M, LoSurdo P, Calzetta A, Noto A, Baysarowich J, Mattu M, Talamo F, De Francesco R, Sparrow C P, Sitlani A, and Carfi A. Structural and Biochemical Characterization of the Wild Type PCSK9-EGF(AB) Complex and Natural Familial Hypercholesterolemia Mutants. J Biol Chem 284(2): 1313-1323 (2009).
Chan J C Y, Piper D E, Cao Q, Liu D, King C, Wang W, Tang J, Liiu Q, Higbee J, Xia Z, Di Y, Shetterly S, Arimura Z, Salomonis H, Romanow W G, Thibault S T, Zhang R, Cao P, Yang X P, Yu T, Lu M, Retter M W, Kwon G, Henne K, Pan O, Tsai M M, Fuchslocher B, Yang E, Zhou L, Lee K J, Daris M, Sheng J, Wang Y, Shen W D, Yeh W C, Emery M, Walker N P C, San B, Schwarz M, and Jackson S M. A proportion convertase subtilisin/kexin type 9 neutralizing antibody reduces serum cholesterol in mice and nonhuman primates. PNAS 106 (24): 9820-9825 (2009).
Ni Y G, Condra J H, Orsatti L, Shen X, Di Marco S, Pandit S, Bottomley M J, Ruggeri L, Cummings R T, Cubbon R M, Santoro J C, Ehrhardt A, Lewis D, Fisher T S, Ha S, Njimoluh L, Wood D D, Hammond H A, Wisniewski D, Volpari C, Noto A, Lo Surdo P, Haubbard B, Carfi A, Sitlani A. A Proprotein Convertase Subtilisin-lik/Kexin Type 9 (PCSK9) C-terminal Domain Antibody Antigen-binding Fragment Inhibits PCSK9 Internalization and Restores Low Density Lipoprotein Uptake. J Biol Chem 285(17): 12882-12891 (2010).
Ni Y G, Di Marco S, Condra J H, Peterson L B, Wang W, Wang F, Pandit S, Hammond H A, Rosa R, Cummings R T, Wood D D, Liu X, Bottomley M J, Shen X, Cubbon R M, Wang S, Johns D G, Volpari C, Hamuro L, Chin J, Huang L, Zhao J Z, Vitelli S, Haytoko P, Wisniewski D, Mitnaul L J, Sparrow C P, Hubbard B, Carfi A, Sitlani A. A PCSK9-binding antibody that structurally mimics the EGF(A) domain of LDL-receptor reduces LDL cholesterol in vivo. J of Lipid Research 52: 78-86 (2011).
Yamamoto T, Lu C, and Ryan R O. A Two-step Binding Model of PCSK9 interaction with the Low Density Lipoprotein Receptor. J Biol Chem. 286(7): 5464-5470 (2011).
Lo Surdo P, Bottomley M J, Calzetta A, Settembre E C, Cirillo A, Pandit S, Ni Y G, Hubbard B, Stilani A, and Carfi A. Mechanistic Implications for LDL receptor degradation from the PCSK9/LDLR structure at neutral pH. EMBO Reports 12 (12): 1300-1305 (2011).
Chaparro-Riggers J, Liang H, DeVay R M, Bai L, Sutton J E, Chen W, Geng T, Lindquist K, Casas M G, Boustany L M, Brown C L, Chabot J, Gomes B, Garzone P, Rossi A, Strop P, Shelton D, Pons J, and Rajpal A. Increasing Serum Half-life and Extending Cholesterol Lowering in vivo by Engineering antibody with pH-sensitive Binding to PCSK9. J Biol Chem 287 (14): 11090-11097 (2012).
Liang H, Chaparro-Riggers J, Strop P, Geng T, Sutton J E, Tsai D, Bai L, Abdiche Y, Dilley J, Yu J, Wu S, Chin S M, Lee N A, Rossi A, Lin J C, Rajpal A, Pons J, and Shelton D L. Proprotein convertase substilisin/kexin type 9 antagonism reduces low-density lipoprotein cholesterol in statin-treated hypercholesterolemic nonhuman primates. J Pharmacol Exp Ther 340(2): 228-236 (2012).
Zhang L, McCabe T, Condra J H, Ni Y G, Peterson L B, Wang W, Strack A M, Wang F, Pandit S, Hammond H, Wood D, Lewis D, Rosa R, Mendoza V, Cumiskey A M, Johns D G, Hansen B C, Shen Xl Geoghagen N, Jensen K, Zhu L, Wietecha K, Wisniewski D, Huang L, Zhao J Z, Ernst R, Hampton R, Haytko P, Ansbro F, Chilewski S, Chin J, Mitnaul L J, Pellacani A, Sparrow C P, An Z, Strhol W, Hubbard B, Plump A S, Blom D, Sitlani A, An Anti-PCSK9 Antibody Reduces LDL-Cholesterol on Top of a Statin and Suppresses Hepatocyte SREBP-Regulated Genes. Int. J. Biol. Sci 8(3): 310-327 (2012).

1. An isolated fusion protein comprising:
a) a first binding domain N-terminal to an immunoglobulin constant region (Fc) domain; and
b) a second binding domain C-terminal the Fc domain;
wherein the first binding domain and the second binding domain are selected from the group consisting of: a proprotein convertase subtilisin/kexin type 9 (PCSK9) inhibitor and a CD40 extracellular domain (CD40 EC).

2. The isolated fusion protein of claim 1, wherein the PCSK9 inhibitor is selected from the group consisting of: a low-density lipoprotein receptor epidermal growth factor-like repeat AB domain (LDLR EGF-AB) and an antigen binding fragment from a PCSK9 binding antibody.

3. The isolated fusion protein of claim 1, wherein the Fc domain is selected from the group consisting of: wildtype human IgG1 Fc domain, mutant human IgG1 Fc domain, wildtype human IgG4 Fc domain, mutant human IgG4 Fc domain.

4. The isolated fusion protein of claim 1, wherein the Fc domain comprises a mutant human IgG1 Fc domain comprising amino acid changes of C220S, C226S and C229S relative to the wildtype human IgG1 Fc domain and amino acid changes of P238S and P331S relative to the wildtype human IgG1 Fc domain.

5. The isolated fusion protein of claim 3, wherein the PCSK9 inhibitor comprises the LDLR EGF-AB domain, wherein the LDLR EGF-AB domain comprises wildtype or mutant human LDLR EGF-AB domain.

6. The isolated fusion protein of claim 5, wherein the LDLR EGF-AB domain comprises a mutant human LDLR EGF-AB domain having an amino acid change of H306Y relative to the wildtype human low-density lipoprotein receptor.

7. The isolated fusion protein of claim 1, wherein the CD40 EC domain comprises wildtype or mutant human CD40 EC domain.

8. The isolated fusion protein of claim 7, wherein the CD40 EC domain comprises a mutant human CD40 EC domain selected from C-terminal truncations at amino acid 145 (P145), at amino acid 188 (P188), at amino acid 190 (D190), as numbered relative to the wildtype human CD40 (SEQ ID NO: 40), and/or one or more amino acid substitutions at K46, E64, E66, D69, E74, H76, Q79, K81, D84, P85, N86, Q93, H110, T112, E114, A115, E117 or L121 relative to the wildtype human CD40 (SEQ ID NO: 40).

9. The isolated fusion protein of claim 8, wherein the mutant human CD40 EC domain comprises one or more amino acid substitutions of K46H, K46T, E64Y, E64S, E66T, D69Q, E74T, H76Q, K81S, K81H, K81T, K81R, P85Y, P85W, N86T, N86Q, Q93S, T112Y, T112S, T112K, E114N, E114R, A115V, E117Q and L121P.

10. The isolated fusion protein of claim 9, wherein the mutant human CD40 EC domain comprises one or more mutations selected from the group consisting of:
(i) E64S;
(ii) E64Y;
(iii) E66T;
(iv) K81S;
(v) K81T;
(vi) T112Y;
(vii) E64S and K81S;
(viii) K81H and L121P;
(ix) E114N and E117Q;
(x) E64Y, K81T and P85Y;
(xi) E64S, K81H and L121P; and
(xii) E64Y, K81T and P85Y.

11. The isolated fusion protein of claim 1, further comprising a third binding domain, wherein the third binding domain comprises a Cytotoxic T-Lymphocyte Antigen 4 (CTLA4).

12. An isolated nucleic acid encoding the isolated fusion protein of claim 1.

13. A recombinant expression vector comprising the nucleic acid of claim 12.

14. A host cell comprising the recombinant expression vector of claim 15.

15. A method for producing the isolated fusion protein of claim 1, comprising:
(a) culturing the host cell of claim 14 under conditions suitable for expression of the nucleic-acid encoded fusion protein; and
(b) isolating the fusion protein from the cultured cells.

16. A pharmaceutical composition, comprising
(a) the isolated fusion protein of claim 1; and
(b) a pharmaceutically acceptable carrier.

17. A method for treating coronary artery disease, comprising administering to a subject in need thereof the isolated fusion protein of claim 1, wherein the fusion protein is administered in an amount effective to treat coronary artery disease.

18. An isolated mutant human CD40 EC domain protein comprising one or more amino acid substitutions selected from the group consisting of: K46H, K46T, E64Y, E64S, E66T, D69Q, E74T, H76Q, K81S, K81H, K81T, K81R, P85Y, P85W, N86T, N86Q, Q93S, T112Y, T112S, T112K, E114N, E114R, A115V, E117Q and L121P relative to the wildtype human CD40.

19. The isolated protein of claim 18, wherein the mutant human CD40 EC domain comprises one or more mutations selected from the group consisting of:
(i) E64S;
(ii) E64Y;
(iii) E66T;
(iv) K81S;
(v) K81T;
(vi) T112Y;
(vii) E64S and K81S;
(viii) K81H and L121P;
(ix) E114N and E117Q;
(x) E64Y, K81T and P85Y;
(xi) E64S, K81H and L121P; and
(xii) E64Y, K81T and P85Y.

20. An isolated nucleic acid encoding the isolated mutant human CD40 EC domain of claim 18.

21. A recombinant expression vector comprising the nucleic acid of claim 20.

22. A host cell comprising the recombinant expression vector of claim 21.

23. A method for producing the isolated mutant human CD40 EC domain of claim 18, comprising:
(a) culturing the host cell of claim 22 under conditions suitable for expression of the nucleic-acid encoded protein; and
(b) isolating the protein from the cultured cells.

24. A pharmaceutical composition, comprising
(a) the isolated mutant human CD40 EC domain of claim 18; and
(b) a pharmaceutically acceptable carrier.

25. A method for treating atherosclerosis in a subject, comprising administering to a subject in need thereof the isolated mutant human CD40 EC domain of claim 18, wherein the CD40 EC domain of claim 18 is administered in an amount effective to inhibit atherosclerotic plaque destabilization.

26. A method for treating an autoimmune disease in a subject, comprising administering to a subject in need thereof isolated mutant human CD40 EC domain of claim 18, wherein the CD40 EC domain is administered in an amount effective to inhibit an autoimmune response.
